(19) United States
(12) Patent Application Publication
(10) Pub. No.: US 2024/0299169 A1
(43) Pub. Date: Sep. 12, 2024

(54) HEART VALVE SEALING DEVICES AND DELIVERY DEVICES THEREFOR

(71) Applicant: EDWARDS LIFESCIENCES CORPORATION, Irvine, CA (US)

(72) Inventors: Eric Michael Oberwise, Newport Beach, CA (US); Michael J. Popp, Irvine, CA (US); Itay Avinathan, Tel-Aviv (IL); David Maimon, Atlit (IL)

(21) Appl. No.: 18/659,509

(22) Filed: May 9, 2024

Related U.S. Application Data
(63) Continuation of application No. PCT/US2022/049305, filed on Nov. 8, 2022.
(60) Provisional application No. 63/279,012, filed on Nov. 12, 2021.

(51) Int. Cl. A61F 2/24

(52) U.S. Cl. CPC A61F 2/2454; A61F 2220/0091; A61F 2230/0043

(57) ABSTRACT

Valve repair devices are disclosed herein. The valve repair devices are configured to reduce or inhibit regurgitant blood flow through a native heart valve. The valve repair devices are configured to be positioned within the native heart valve orifice and attached to the native heart valve. The device can be connected to leaflets of the native valve by a variety of different types of paddles.

### RELATED APPLICATIONS

This present application is a continuation of Patent Cooperation Treaty Application No. PCT/US2022/049305, filed on Nov. 8, 2022, which claims the benefit of U.S. Provisional Application No. 63/279,012, filed on Nov. 12, 2021, the entire disclosures of which are fully incorporated by reference.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation can have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation can be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, valve repair devices are configured to reduce or inhibit regurgitant blood flow through a native heart valve. The valve repair devices are configured to be positioned within the native heart valve orifice and attached to the native heart valve. The device can be connected to leaflets of the native valve by a variety of different types of anchors. The anchors can comprise a variety of different types of paddles.

In some implementations, a valve repair device for repairing a native valve of a patient has a coaptation element formed from a solid or hollow piece of molded material, a paddle portion with a plurality of paddles that are movable between an open position and a closed position, and an attachment portion having a collar and two clasp elements, each clasp element having a clasp securing recess. The paddle portion is configured to attach to the native valve of the patient and hold leaflets of the native valve against the attachment portion. The paddles can be moved between the open position and the closed position independently.

In some implementations, the paddle portion is secured in paddle securing recesses of the coaptation element.

In some implementations, the attachment portion is secured in clasp securing recesses of the coaptation element.

In some implementations, the attachment portion is formed from a super-elastic sheet.

In some implementations, the valve repair device includes a biasing element which biases one of the paddles to one of the open and closed positions.

In some implementations, the valve repair device includes a connection element configured to move one of the paddles between the opened and closed positions. The paddle can include a connection portion for connecting to the connection element.

In some implementations, the paddle portion includes an outer paddle and an inner paddle.

In some implementations, a valve repair system and/or device for repairing a native valve of a patient has a coaptation element formed from a solid or hollow piece of molded material and having a passage, a paddle portion having a plurality of paddles that are movable between an open position and a closed position, and an attachment portion having a collar and two clasp elements.

In some implementations, the paddle portion is configured to attach to the native valve of the patient and hold leaflets of the native valve against the attachment portion.

In some implementations, the paddles can be moved between the open position and the closed position independently.

In some implementations, the coaptation element further comprises a biasing feature or biasing element for engaging a paddle extension shaft of the paddle. The biasing feature or biasing element can bias the paddle to the closed position.

In some implementations, the paddle is movable to the open position with an actuation element.

In some implementations, the paddle portion is formed from a single super-elastic sheet.

In some implementations, the coaptation element further comprises a passage.

In some implementations, a valve repair system and/or device for repairing a native valve (e.g., of a patient or simulation) has a coaptation element having two actuators and an anchor portion. In some implementations, the anchor portion comprises a paddle portion having a plurality of paddles that are movable between an open position and a closed position.

In some implementations, the valve repair system/device includes an attachment portion.

In some implementations the anchor portion (e.g., the paddle portion) is configured to attach to the native valve of the patient and hold leaflets of the native valve against the attachment portion.

In some implementations, the plurality of paddles can be moved between the open position and the closed position independently.

In some implementations, distal movement of one of the actuators causes one of the paddles to move to the open position, and proximal movement of the one actuator causes one of the paddles to move to the closed position.

In some implementations, each actuator is connected to one of the paddles by a connection element.

In some implementations, movement of each of the paddles is controlled by a biasing element.

In some implementations, the paddles are biased distally.

In some implementations, the paddle portion is formed from a single super-elastic sheet.

In some implementations, the attachment portion is biased distally.

In some implementations, a valve repair system and/or device for repairing a native valve of a patient includes a first retaining hinge, a second retaining hinge, and a paddle. In some implementations, the second retaining hinge is disposed proximally to the first retaining hinge.

In some implementations, the paddle includes a paddle arm and a follower arm.

In some implementations, the paddle arm has a first paddle member with a stop and a paddle fastener rotatably retained in the first retaining hinge.

In some implementations, the follower arm has a follower fastener rotatably retained in the second retaining hinge and a paddle connector slidable along a portion of the first paddle member.

In some implementations, the paddle is rotatable from an open position to a first position in which the paddle connector abuts the stop, a center position in which the paddle arm and the follower arm are substantially aligned, and a closed position. At least one of the first retaining hinge, the second retaining hinge, and the follower arm bias the paddle arm to the closed position when the paddle is rotated past the center position.

In some implementations, at least one of the first retaining hinge and the second retaining hinge bias the paddle arm to the closed position when the paddle is rotated past the center position.

In some implementations, a single one of the first retaining hinge and the second retaining hinge biases the paddle arm to the closed position when the paddle is rotated past the center position.

In some implementations, the second retaining hinge biases the paddle arm to the closed position when the paddle is rotated past the center position.

In some implementations, the paddle arm further comprises a second paddle member disposed opposite the first paddle member.

In some implementations, the first paddle member is a wire loop, and the stop comprises a rod disposed between legs of the first paddle member.

In some implementations, at least one of the paddle arm and the follower arm comprise Nitinol.

In some implementations, the paddle further comprises a gripping member with a movable arm that is movable between a closed position and an open position.

In some implementations, the gripping member further comprises a collar disposed around the coaptation element and a joint portion between the collar and the movable arm, wherein the joint portion biases the movable arm to the closed position.

In some implementations, the gripping member further comprises a fixed arm attached to the first paddle member and a joint portion between the fixed arm and the movable arm, wherein the joint portion biases the movable arm to the closed position.

In some implementations, the second paddle member is disposed at an obtuse angle from the first paddle member.

In some implementations, the follower arm exerts a leaf spring biasing force on the paddle when the paddle is rotated proximally past the first point.

In some implementations, the valve repair system/device includes a coaptation element attached to the first retaining hinge and the second retaining hinge.

In some implementations, a valve repair system and/or device includes a base and a paddle. The paddle can include a paddle arm and a paddle arm connector.

In some implementations, the paddle arm has a first leg portion having a first connecting portion and a second leg having a second connecting portion.

In some implementations, the paddle arm connector has a fixed retaining portion for receiving the second connecting portion and first and second receiving portions for receiving the first connecting portion.

In some implementations, the paddle arm is rotatable about the paddle arm connector when the first connecting portion is disposed in the first receiving portion and is biased against rotation when the first connecting portion is disposed in the second receiving portion.

In some implementations, the paddle arm connector includes a channel connecting the first and second receiving portions.

In some implementations, the first receiving portion and the fixed retaining portion are disposed at a first height and the second receiving portion is disposed at a second height, the second height being greater than the first height.

In some implementations, the channel is L-shaped.

In some implementations, the channel includes a first channel portion extending upwardly from the first receiving portion, a second channel portion extending laterally from the first channel portion, and a third channel portion extending downwardly from an end of the second channel portion opposite the first channel portion to the second receiving portion.

In some implementations, the first channel portion extends to a third height greater than the second height.

In some implementations, the paddle arm comprises Nitinol.

In some implementations, a force required to rotate the paddle arm when the first receiving portion is disposed in the second receiving portion is proportional to the amount the paddle arm is rotated about the paddle arm connector.

In some implementations, the base comprises a coaptation element.

In some implementations, any of the devices herein can be part of a valve repair system that includes a delivery system and the device (e.g., a valve repair device, etc.).

In some implementations, the valve repair system and/or device is sterilized.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of implementations of the present disclosure, a more particular description of certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a front view of the implantable device or implant of FIG. 22;

FIG. 24 shows a side view of the implantable device or implant of FIG. 22;

FIG. 25 shows a front view of the implantable device or implant of FIG. 22 with a cover covering the paddles and a coaptation element or spacer;

FIG. 26 shows a top perspective view of the implantable device or implant of FIG. 22 in an open position;

FIG. 27 shows a bottom perspective view of the implantable device or implant of FIG. 22 in an open position;

FIG. 28A shows a clasp for use in an implantable device or implant;

FIG. 28B shows a perspective view of an example clasp of an example implantable device or implant in a closed position;

FIG. 29 shows a portion of native valve tissue grasped by a clasp;

FIG. 30 shows a side view of an example implantable device or implant in a partially open position with clasps in a closed position;

FIG. 31 shows a side view of an example implantable device or implant in a partially open position with clasps in an open position;

FIG. 32 shows a side view of an example implantable device or implant in a half-open position with clasps in a closed position;

FIG. 33 shows a side view of an example implantable device or implant in a half-open position with clasps in an open position;

FIG. 34 shows a side view of an example implantable device or implant in a three-quarters-open position with clasps in a closed position;

FIG. 35 shows a side view of an example implantable device or implant in a three-quarters-open position with clasps in an open position;

FIG. 36 shows a side view of an example implantable device in a fully open or full bailout position with clasps in a closed position;

FIG. 37 shows a side view of an example implantable device in a fully open or full bailout position with clasps in an open position;

FIGS. 38-49 show the example implantable device or implant of FIGS. 30-38, including a cover, being delivered and implanted within a native valve;

FIG. 50 is a schematic view illustrating a path of native valve leaflets along each side of a coaptation element or spacer of an example valve repair device or implant;

FIG. 51 is a top schematic view illustrating a path of native valve leaflets around a coaptation element or spacer of an example valve repair device or implant;

FIG. 52 illustrates a coaptation element or spacer in a gap of a native valve as viewed from an atrial side of the native valve;

FIG. 53 illustrates a valve repair device or implant attached to native valve leaflets with the coaptation element or spacer in the gap of the native valve as viewed from a ventricular side of the native valve;

FIG. 54 is a perspective view of a valve repair device or implant attached to native valve leaflets with the coaptation element or spacer in the gap of the native valve shown from a ventricular side of the native valve;

FIG. 55 shows a perspective view of an example implantable device or implant in a closed position;

FIG. 56A illustrates a valve repair device with paddles in an open position;

FIG. 56B illustrates the valve repair device of FIG. 56A, in which the paddles are in the open position and gripping members are moved to create a wider gap between the gripping members and paddles;

FIG. 56C illustrates the valve repair device of FIG. 56A, in which the valve repair device is in the position shown in FIG. 56A with valve tissue placed between the gripping members and the paddles;

FIG. 56D illustrates the valve repair device of FIG. 56A, in which the gripping members are moved to lessen the gap between the gripping members and the paddles;

FIGS. 56E-56F illustrate the movement of the paddles of the valve repair device of FIG. 56A from the open position to a closed position;

FIG. 56G illustrates the valve repair device of FIG. 56A in a closed position, in which the gripping members are engaging valve tissue;

FIG. 56H illustrates the valve repair device of FIG. 56A after being disconnected from a delivery device and attached to valve tissue, in which the valve repair device is in a closed and locked condition;

FIG. 57 shows an example of an implantable prosthetic device having paddles that are independently controllable;

FIG. 58 shows a front view of the implantable prosthetic device of FIG. 57;

FIGS. 59A-59D show various views of an optional coaptation element for use with the implantable prosthetic device of FIG. 57;

FIGS. 60A-60D show various views of a paddle portion for use with the implantable prosthetic device of FIG. 57;

FIGS. 61A-61D show various views of an attachment portion for use with the implantable prosthetic device of FIG. 57;

FIGS. 62A and 62B show top perspective and front views of the implantable prosthetic device of FIG. 57 with the coaptation element removed;

FIG. 63 is a front exploded view of the implantable prosthetic device of FIG. 57;

FIG. 64 is a top perspective exploded view of the implantable prosthetic device of FIG. 57;

FIGS. 65-67 show front schematic views of the implantable prosthetic device of FIG. 57, in various stages of deployment;

FIG. 68 shows an example of an implantable prosthetic device where the paddles are independently controllable;

FIGS. 69 and 70 show front and side views of the implantable prosthetic device of FIG. 68;

FIGS. 71A and 71B show top and bottom views of the implantable prosthetic device of FIG. 68;

FIGS. 72 and 73 show top perspective and front exploded views of the implantable prosthetic device of FIG. 68;

FIGS. 74A-74D show various views of an optional coaptation element for use with the implantable prosthetic device of FIG. 68;

FIGS. 75A-75C show various views of a paddle portion for use with the implantable prosthetic device of FIG. 68;

FIGS. 76A-76D show various views of an attachment portion for use with the implantable prosthetic device of FIG. 68;

FIGS. 77-79 show front schematic views of the implantable prosthetic device of FIG. 68, in various stages of deployment;

FIGS. 80A and 80B show an example of an implantable prosthetic device where the paddles are independently controllable;

FIGS. 81 and 82 show front and top perspective exploded views of the implantable prosthetic device of FIGS. 80A and 80B;

FIGS. 83A-83E show various views of a coaptation element for use with the implantable prosthetic device of FIGS. 80A and 80B;

FIGS. 84A and 84B are front views of a paddle portion for use with the implantable prosthetic device of FIGS. 80A and 80B in various stages of deployment;

FIGS. 85A and 85B are side views of the paddle portion for use with the implantable prosthetic device of FIGS. 80A and 80B in various stages of deployment;

FIGS. 86A and 86B are top views of the paddle portion for use with the implantable prosthetic device of FIGS. 80A and 80B in various stages of deployment;

FIGS. 87A-87D show various views of an attachment portion for use with the implantable prosthetic device of FIGS. 80A and 80B;

FIGS. 88-94 show front schematic views of the implantable prosthetic device of FIGS. 80A and 80B, in various stages of deployment;

FIGS. 95-98 show schematic views of an example of an implantable device or implant, in various stages of deployment;

FIGS. 99-103 illustrate an example of an implantable device or implant that is similar to the device illustrated in FIGS. 95-98, in various stages of deployment;

FIG. 104 illustrates an example valve repair device or implant that is similar to the device or implant of FIGS. 99-103, but with two paddles;

FIG. 105 illustrates the valve repair device or implant of FIG. 104 where the paddles are independently controllable;

FIG. 106 illustrates the valve repair device or implant of FIG. 104 where the paddles are controllable in unison;

FIG. 107 illustrates an example valve repair device or implant that is similar to the device or implant of FIG. 104, but with an attachment portion or gripping members that are in a closed position;

FIG. 108 illustrates the valve repair device or implant of FIG. 107, but with the gripping members in an open position;

FIG. 109 illustrates an example valve repair device or implant that is similar to the device or implant of FIG. 104, but with gripping members according to another example with the gripping members in a closed position;

FIG. 110 illustrates the valve repair device or implant of FIG. 109 with the gripping elements in an open position;

FIG. 111 shows a schematic view of an implantable device or implant in an open position;

FIG. 112 shows a schematic view of the implantable device or implant of FIG. 111 in a closed position;

FIGS. 113-116 illustrate perspective views of an example paddle of the implantable device of FIG. 110, in various positions;

FIGS. 117A-117C illustrate a force required to rotate a paddle of the valve repair device of FIG. 110 when the paddle is in an unbiased position;

FIG. 118A-118C illustrate a force required to rotate a paddle of the valve repair device of FIG. 110 when the paddle is in a biased position; and

FIGS. 119-124 illustrate the implantable device or implant of FIG. 110, in various stages of deployment in a native heart.

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for repairing a defective heart valve. For example, implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc. As used herein the term “simulation” encompasses simulations performed on a cadaver, a computer simulator, an imaginary person, in open space etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

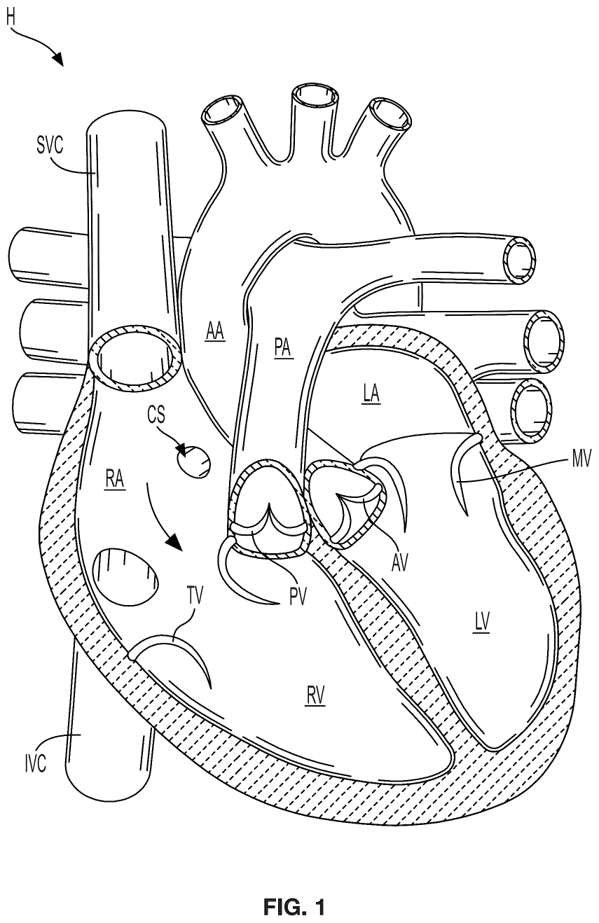

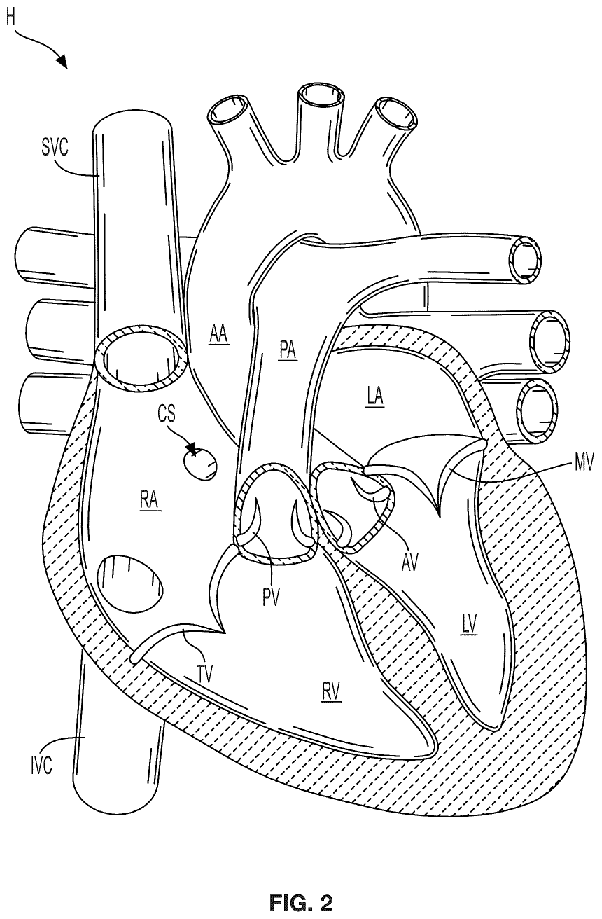

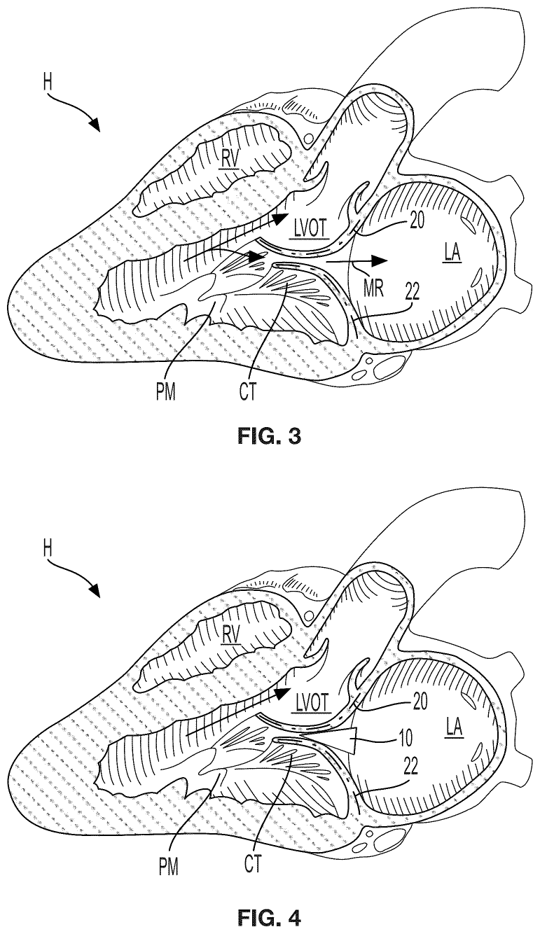

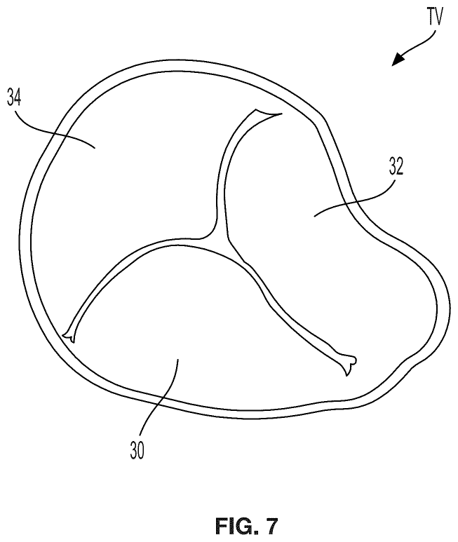

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV, i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit, or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant or incompetent-which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

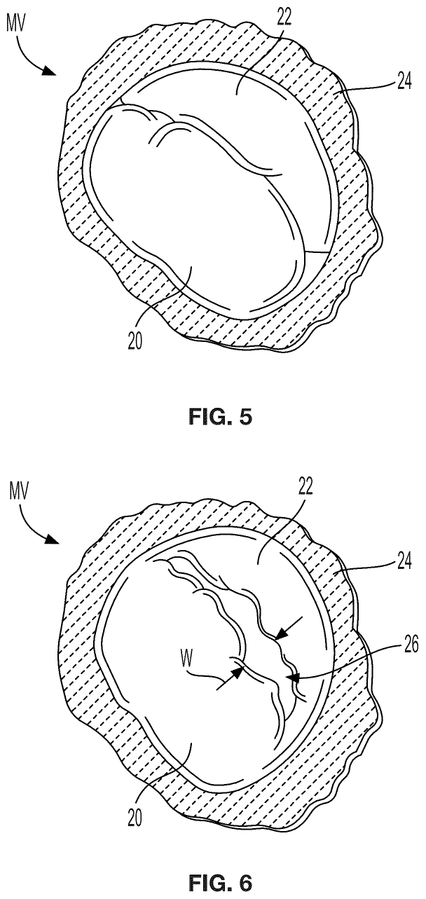

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm or even 17.5 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve repair device, implantable device, or implant 10 is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, and balloon etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaptation element—e.g., spacer, coaption element, gap filler, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., coaption element, spacer, etc.) is configured to be positioned within the native heart valve orifice to help fill the space between the leaflets and form a more effective seal, thereby reducing, preventing, or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral valve leaflets 20, 22 or tricuspid valve leaflets 30, 32, 34) that do not close completely.

The optional coaptation element (e.g., spacer, coaption element, gap filler, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface extends between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft, rod, tube, wire, etc., to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

Referring now to FIGS. 8-15, a schematically illustrated device or implant 100 (e.g., an implantable prosthetic device, a prosthetic spacer device, a valve repair device, an implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion/coaptation region 104 and an anchor portion/anchor region 106.

In some implementations, the coaptation portion 104 of the device or implant 100 includes a coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during implantation. The actuation element 112 (as well as other means for actuating and actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and actuation element 112 (e.g., actuation wire, actuation shaft, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a movable arm 134, optional friction-enhancing elements, or other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate a coaptation element 110. The joint portion 138 provides a spring force between the fixed and movable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and movable arms 132, 134. The fixed arms 132 are attached to the inner paddles 122 and remain stationary or substantially stationary relative to the inner paddles 122 when the movable arms 134 are opened to open the clasps 130 and expose the optional barbs, friction-enhancing elements, or securing structures 136.

In some implementations, the clasps 130 are opened by applying tension to actuation lines 116 attached to the movable arms 134, thereby causing the movable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs, friction-enhancing elements, or securing structures 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, gap filler, etc.). The clasps 130 can be used to grasp and/or further secure the native leaflets by engaging the leaflets with optional barbs, friction-enhancing elements, or securing structures 136 and pinching the leaflets between the movable and fixed arms 134, 132. The optional barbs, friction-enhancing elements, or other securing structures 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

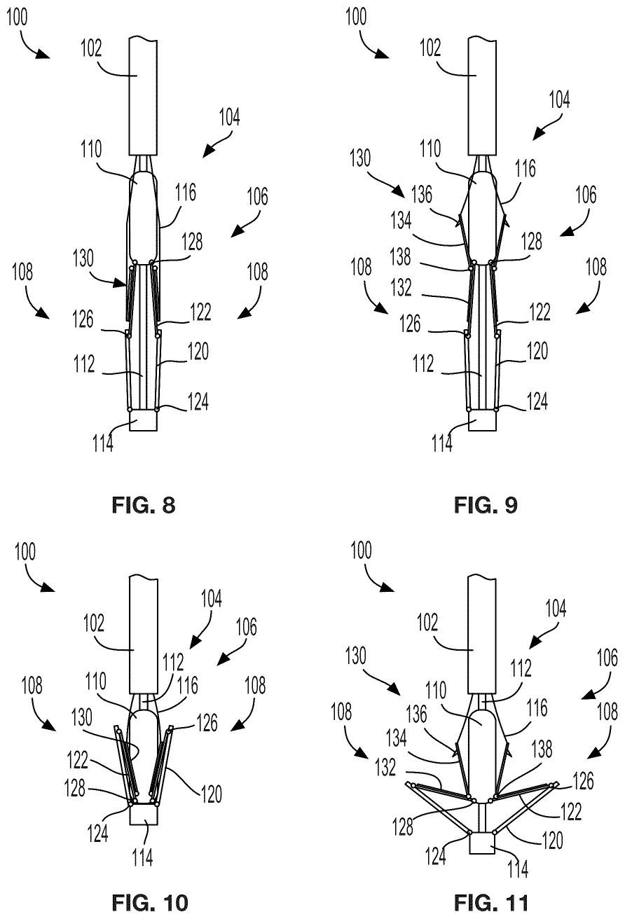

Referring now to FIG. 8, the device 100 is shown in an elongated or fully open condition for deployment from an implant delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system 102, so that the optional barbs, friction-enhancing elements, or other securing structures 136 (FIG. 9) do not catch or damage the delivery system 102. The actuation lines 116 can extend and attach to the movable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and movable portions 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. To move the device 100 from the elongated condition to the shortened condition, the actuation element 112 is retracted to pull the cap 114 towards the coaptation element 110 (e.g., towards a spacer). The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., wire, shaft, etc.) and cap (or other attachment portion) are used to control one paddle, and the other independent actuation element and cap (or other attachment portion) are used to control the other paddle.

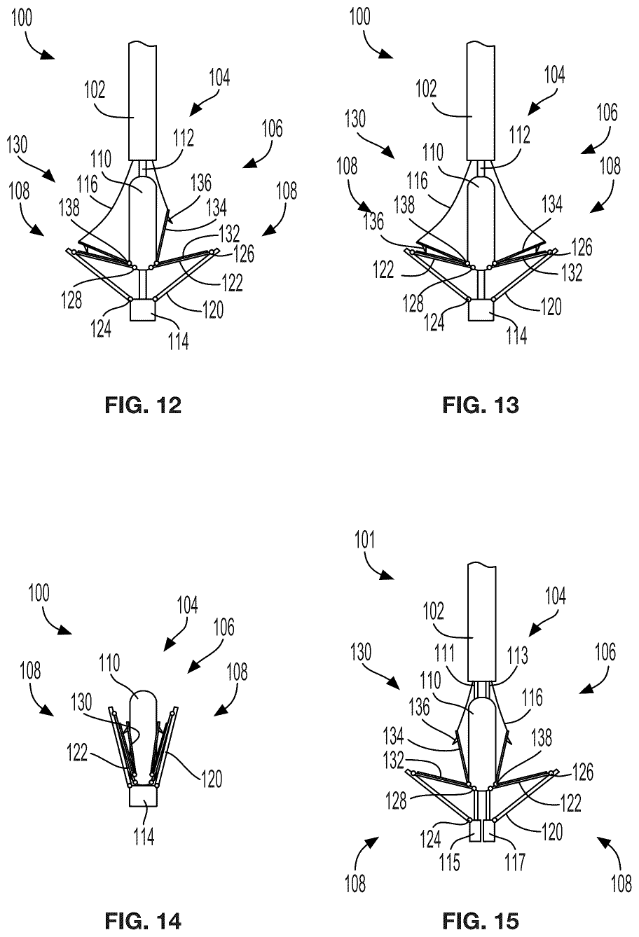

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and movable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements (e.g., actuation shafts, actuation rods, actuation tubes, actuation wires, etc.) 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

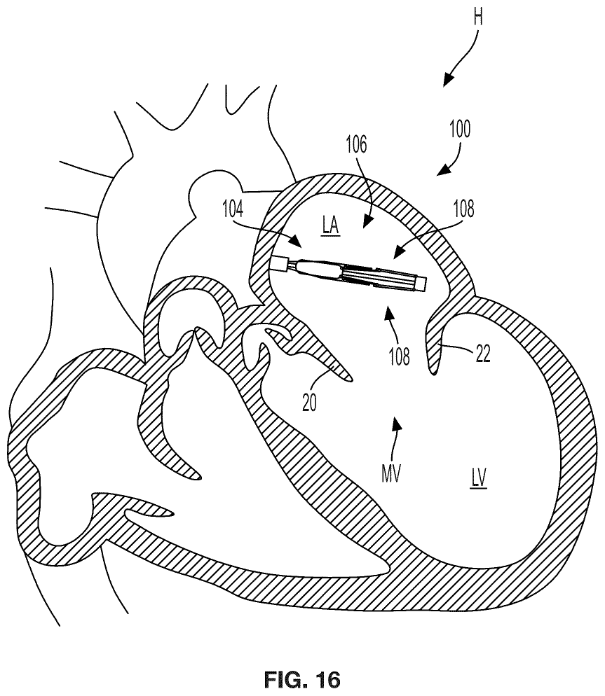

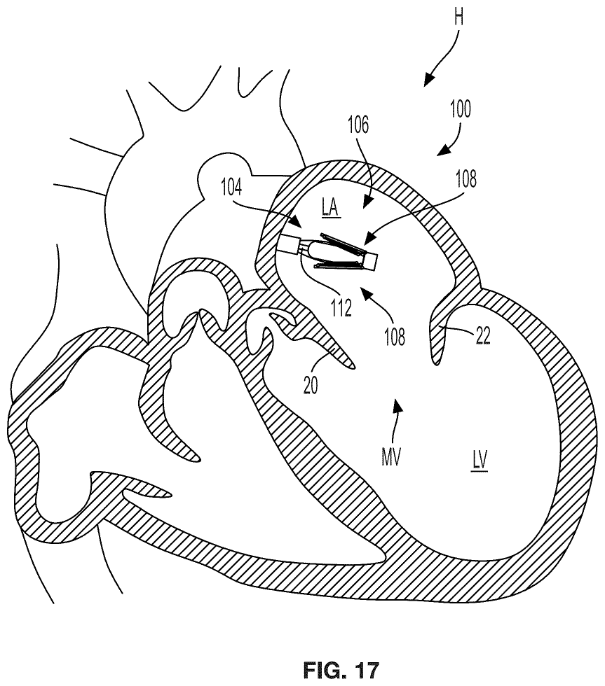

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

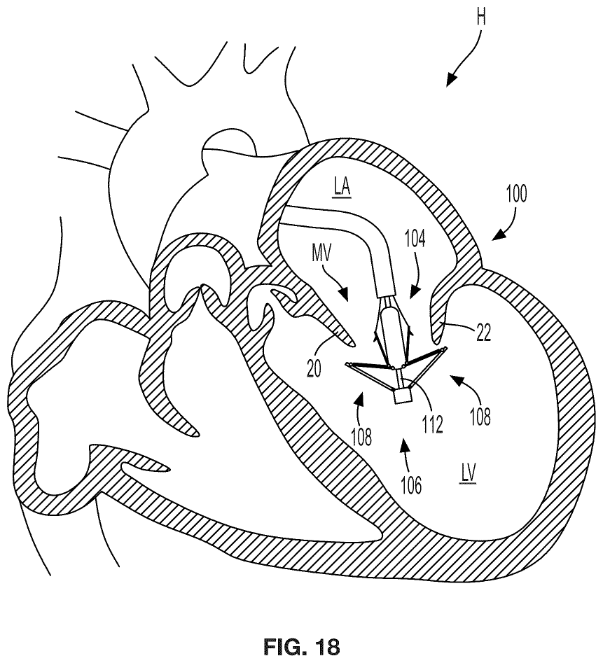

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

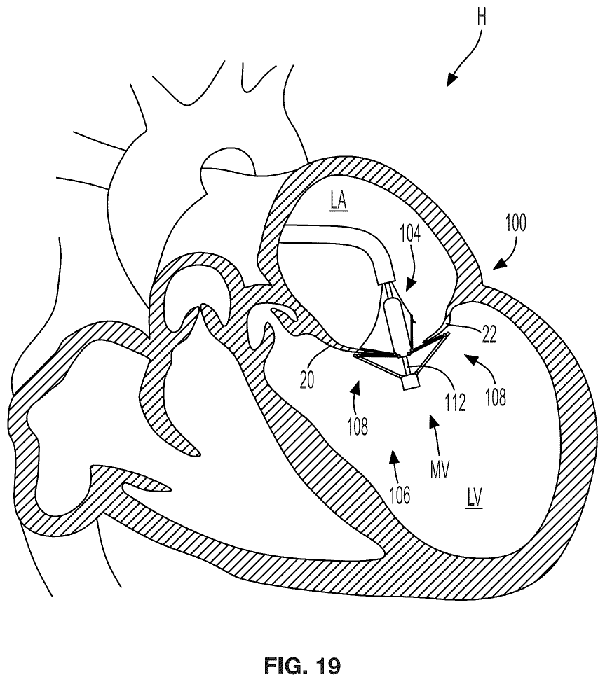

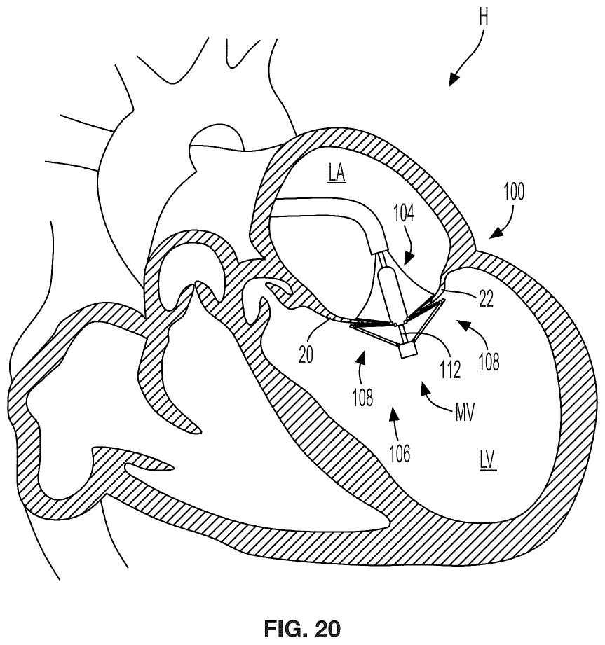

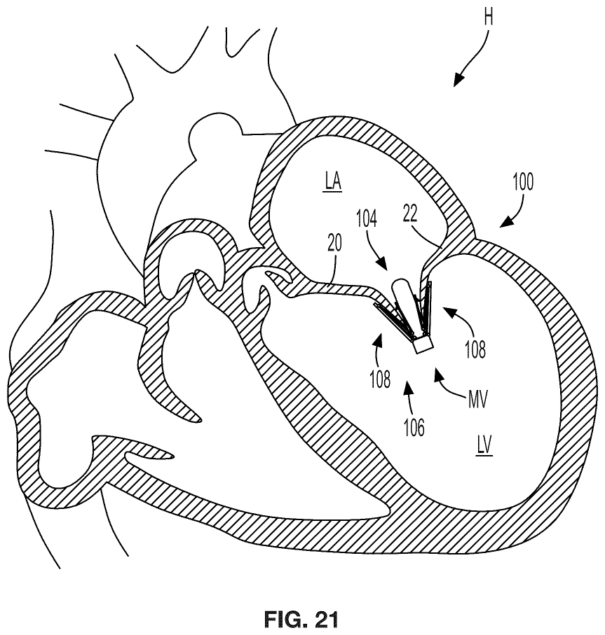

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

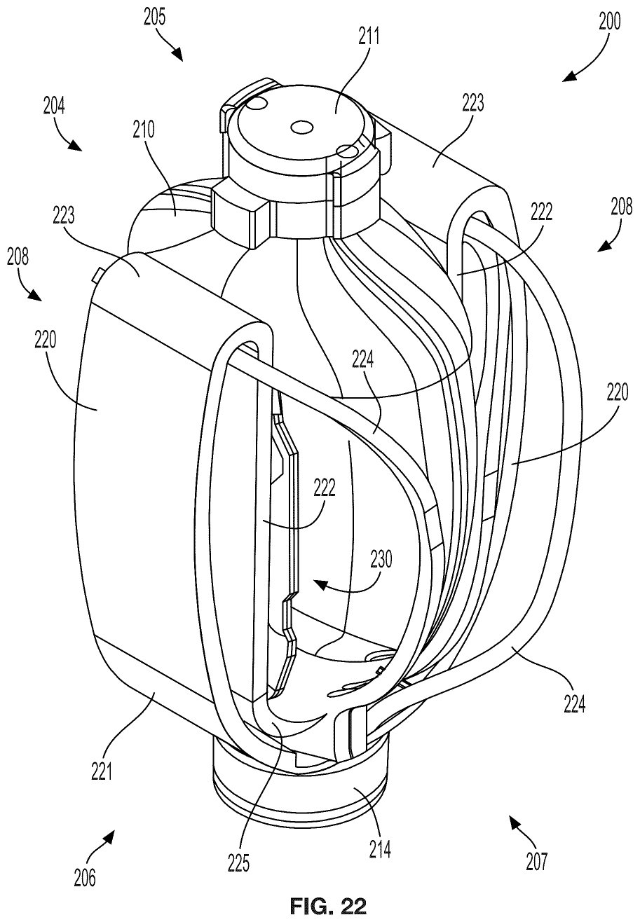

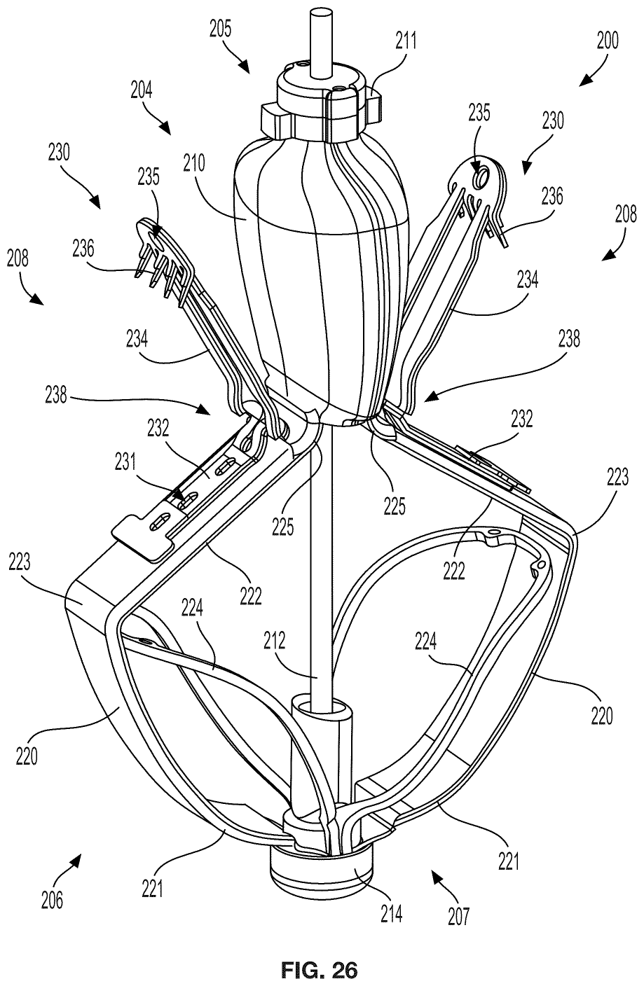

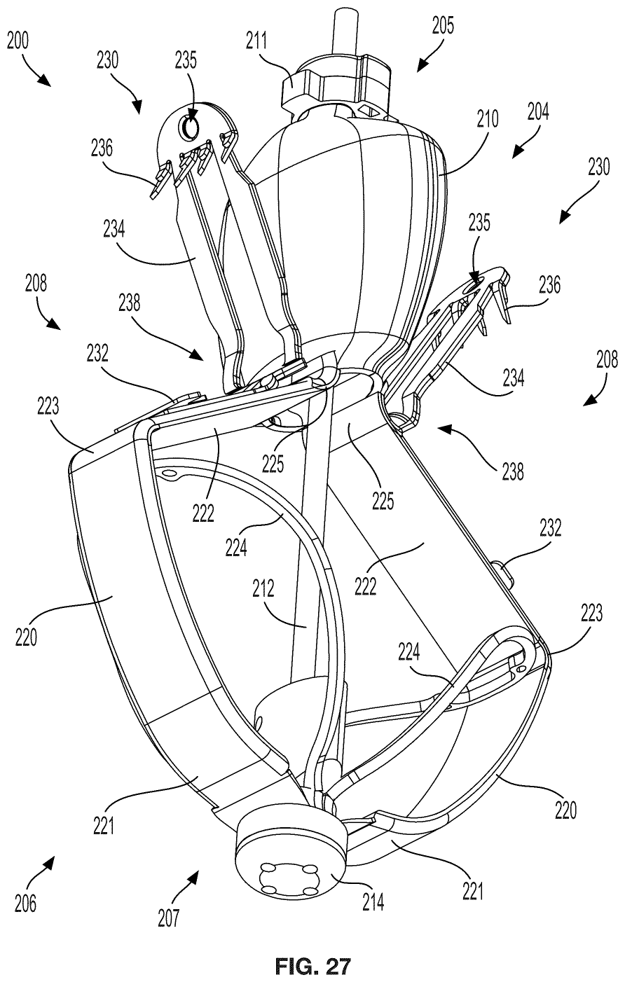

Any of the features disclosed by the present application can be used in a wide variety of different valve repair devices. FIGS. 22-27 and 56A-56H illustrate examples of valve repair devices that can be modified to include any of the features disclosed by the present application. Any combination or sub-combination of the features disclosed by the present application can be combined with, substituted for, and/or added to any combination or sub-combination of the features of the valve repair devices illustrated by FIGS. 22-27 and 56A-56H.

Referring now to FIG. 22, an example of an implantable device or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a valve repair device, implantable device, or another type of implant that attaches to leaflets of a native valve.

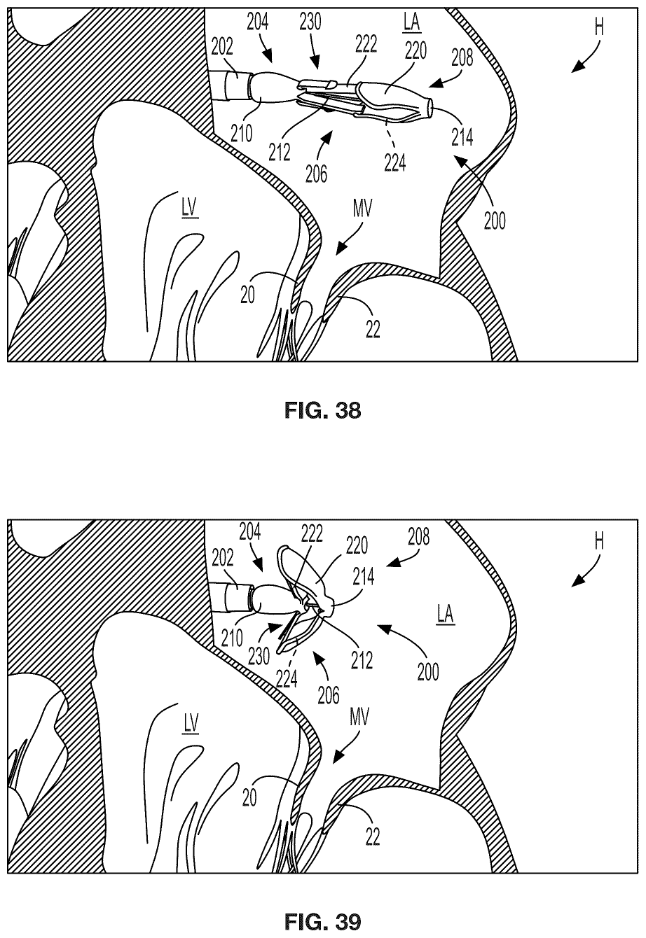

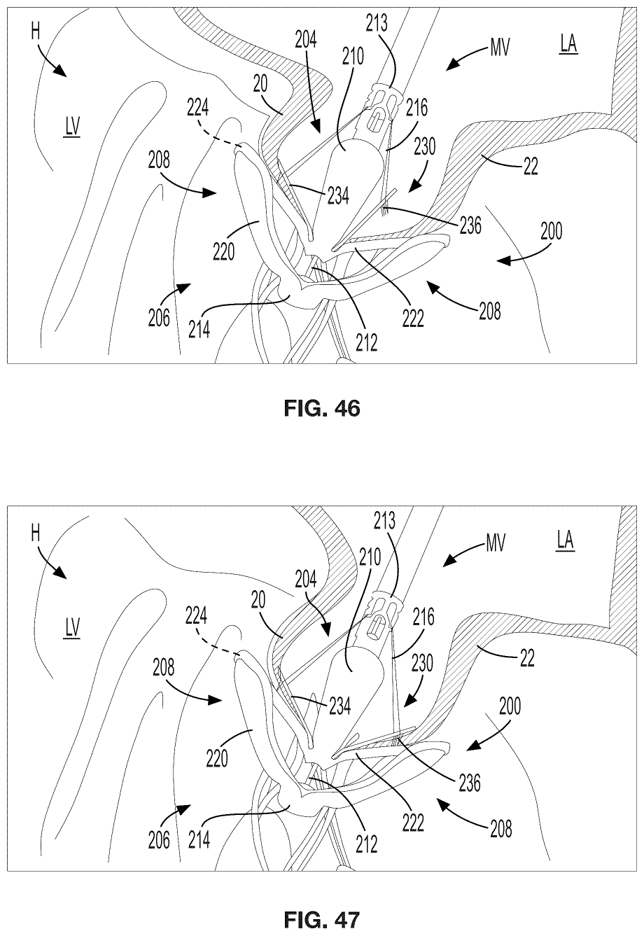

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism 213 (see e.g., FIGS. 43-49) of a delivery system 202 (see e.g., FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the implantable device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

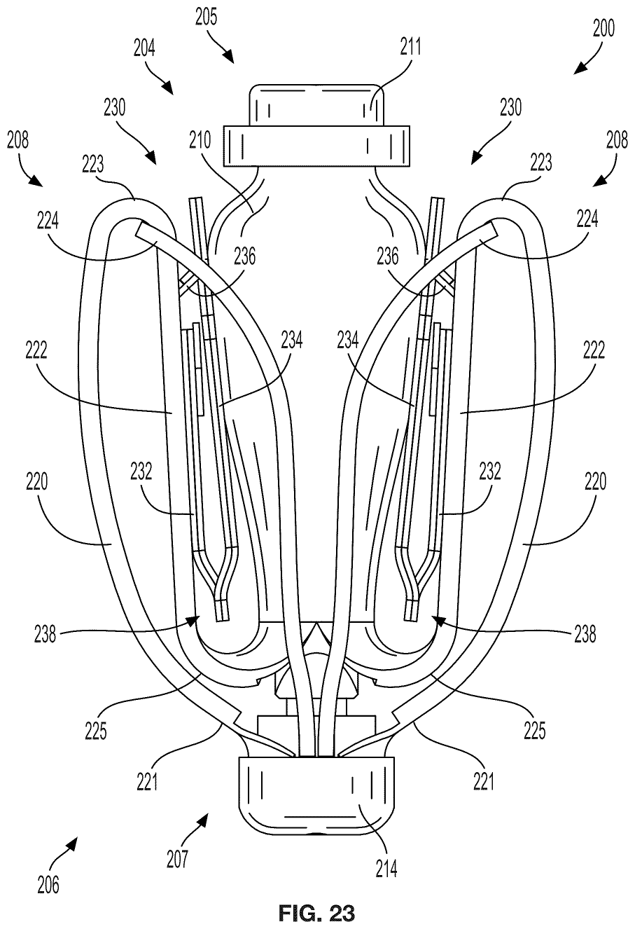

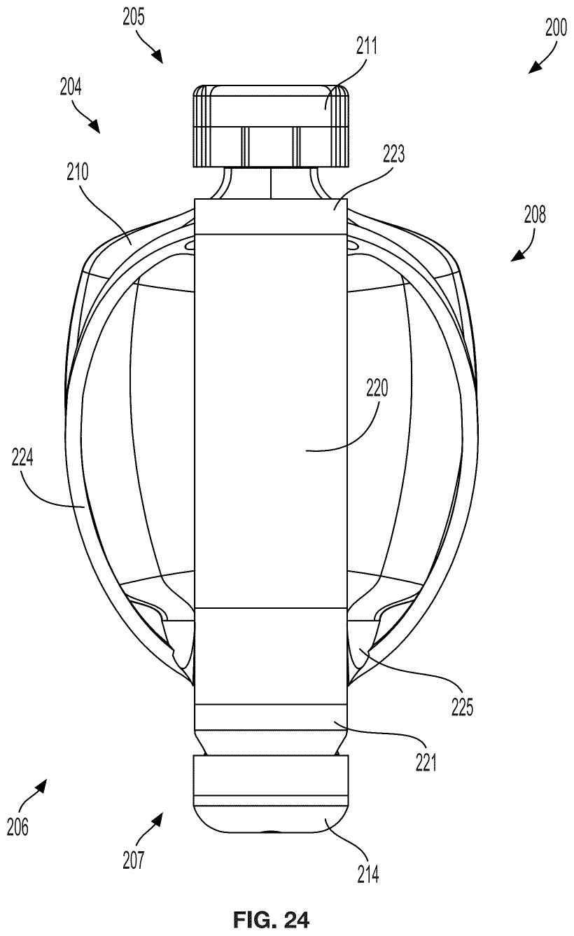

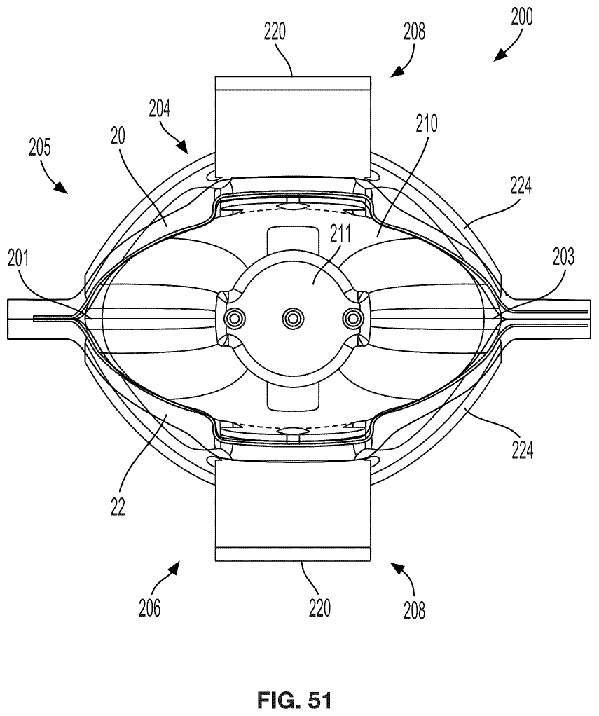

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the implantable device or implant 200, such that the native leaflets coapt entirely around the coaptation member or coaptation element 210. This can, for example, improve sealing of the native leaflets 20, 22 and thus prevent, inhibit, or further reduce mitral regurgitation.

In some implementations the clasps comprise a movable arm coupled to the anchors. In some implementations, the clasps 230 include a base or fixed arm 232, a movable arm 234, with optional barbs, friction-enhancing elements, or securing structures 236, and a joint portion 238. The fixed arms 232 are attached to the inner paddles 222, with the joint portion 238 disposed proximate the coaptation element 210. The joint portion 238 is spring-loaded so that the fixed and movable arms 232, 234 are biased toward each other when the clasp 230 is in a closed condition. In some implementations, the clasps 230 include friction-enhancing elements or means for securing, such as barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the movable arms 234 are opened to open the clasps 230 and expose the optional barbs, friction-enhancing elements, or securing structures 236. The clasps 230 are opened by applying tension to actuation lines 216 (e.g., as shown in FIGS. 43-48) attached to holes 235 in the movable arms 234, thereby causing the movable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the movable and fixed arms 232, 234 of the clasp 230. The tissue of the leaflet 20, 22 is not pierced by the optional barbs, friction-enhancing elements, or securing structures 236, though in some implementations the optional barbs 236 can partially or fully pierce through the leaflet 20, 22. The angle and height of the optional barbs, friction-enhancing elements or securing structures 236 relative to the movable arm 234 helps to secure the leaflet 20, 22 within the clasp 230. In particular, a force pulling the implant off of the native leaflet 20, 22 will encourage the optional barbs, friction-enhancing elements, or securing structures 236 to further engage the tissue, thereby ensuring better retention. Retention of the leaflet 20, 22 in the clasp 230 is further improved by the position of fixed arm 232 near the optional barbs, friction-enhancing elements, or securing structures 236 when the clasp 230 is closed. In this arrangement, the tissue is formed by the fixed arms 232 and the movable arms 234 and the optional barbs, friction-enhancing elements, or securing structures 236 into an S-shaped torturous path. Thus, forces pulling the leaflet 20, 22 away from the clasp 230 will encourage the tissue to further engage the optional barbs, friction-enhancing elements, or securing structures 236 before the leaflets 20, 22 can escape. For example, leaflet tension during diastole can encourage the optional barbs, friction-enhancing elements, or securing structures 236 to pull toward the end portion of the leaflet 20, 22. Thus, the S-shaped path can utilize the leaflet tension during diastole to engage the leaflets 20, 22 more tightly with the optional barbs, friction-enhancing elements or securing structures 236.

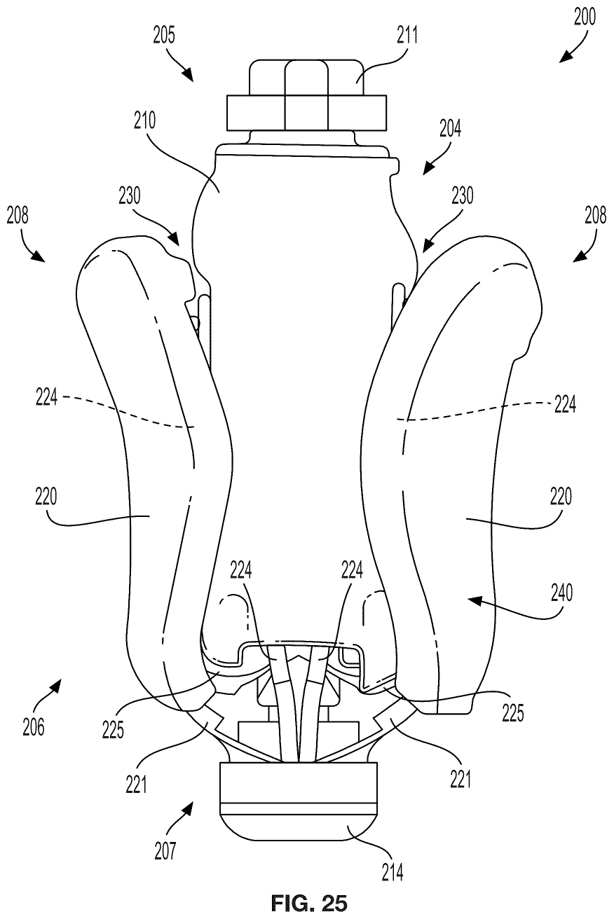

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the implantable device or implant 200.

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 220 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 100 illustrated in FIG. 15).

In some implementations, the clasps 230 further secure the native leaflets 20, 22 by engaging the leaflets 20, 22 with optional barbs, friction-enhancing elements, or securing structures 236 and/or pinching the leaflets 20, 22 between the movable and fixed arms 234, 232. In some implementations, the clasps 230 are barbed clasps that include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to grip the leaflets 20, 22 more securely when the device 200 is closed (e.g., as can be seen in FIG. 51). The curved shape and rounded edges of the outer paddle 220 also prohibits or inhibits tearing of the leaflet tissue.

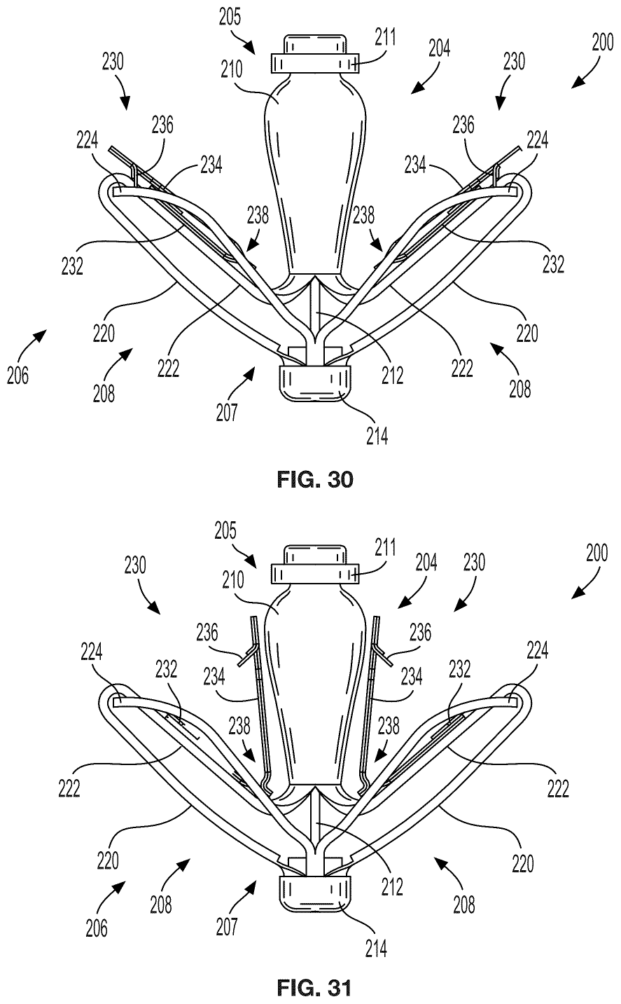

Referring now to FIGS. 30-37, the implantable device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and movable arms 232, 234 of the clasp 230 is limited to the extent that the inner paddle 222 has spread away from the coaptation element 210.

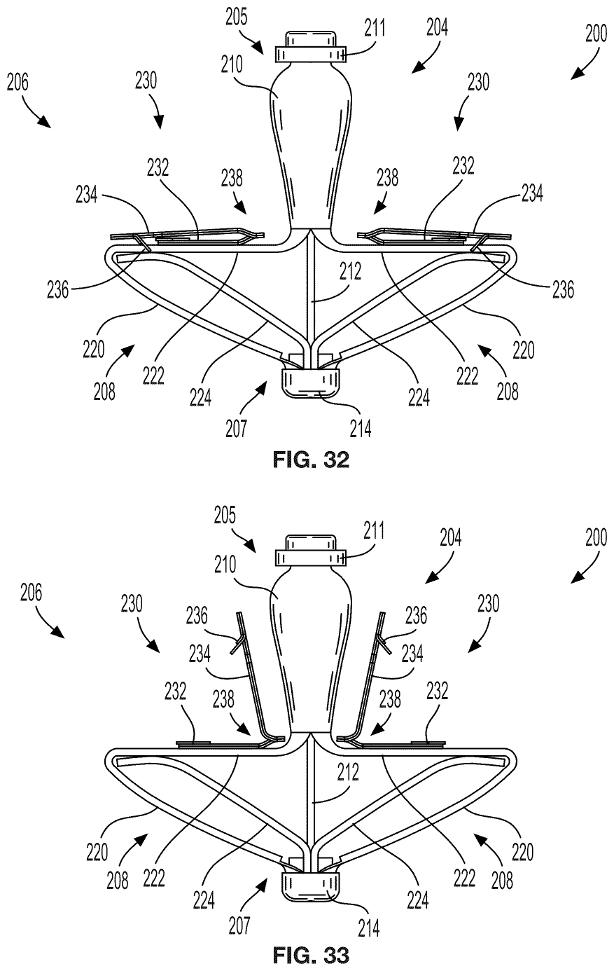

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and movable arms 232, 234.

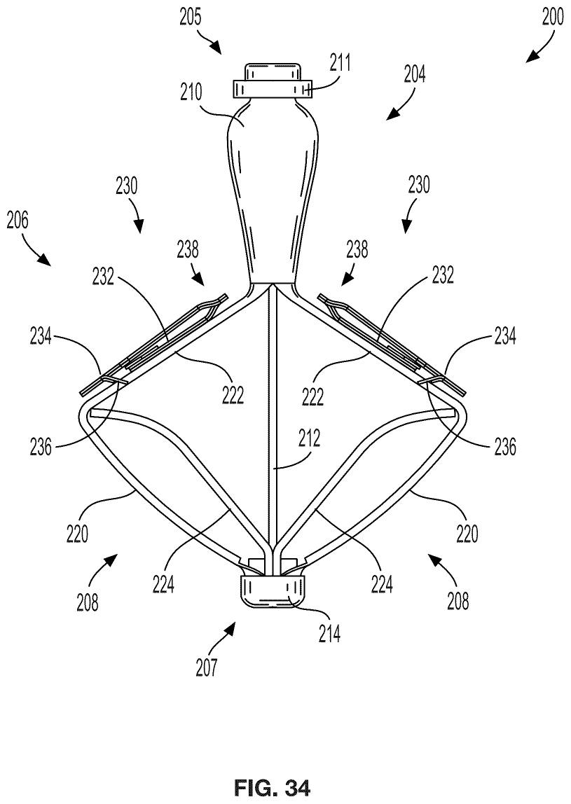

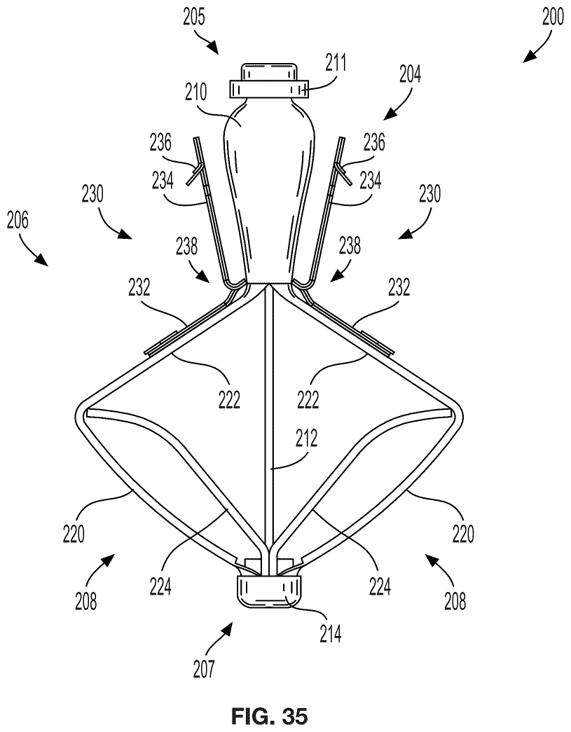

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and movable arms 232, 234.

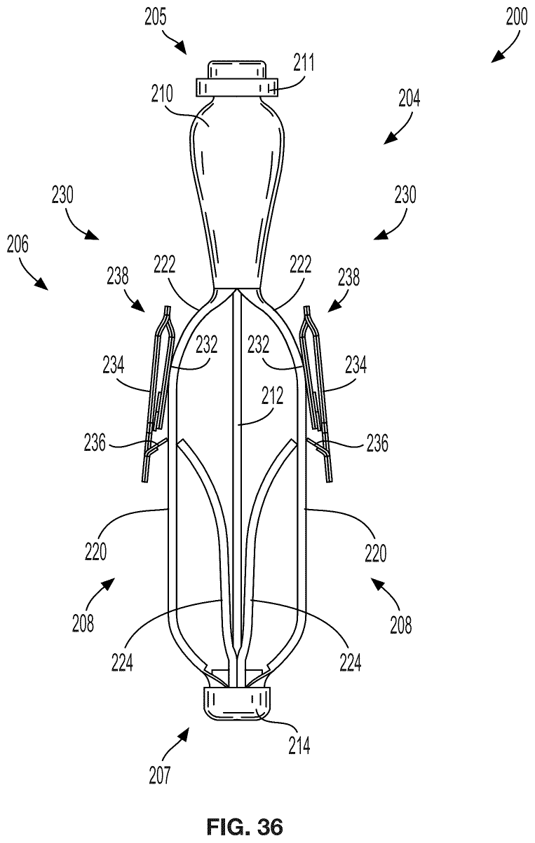

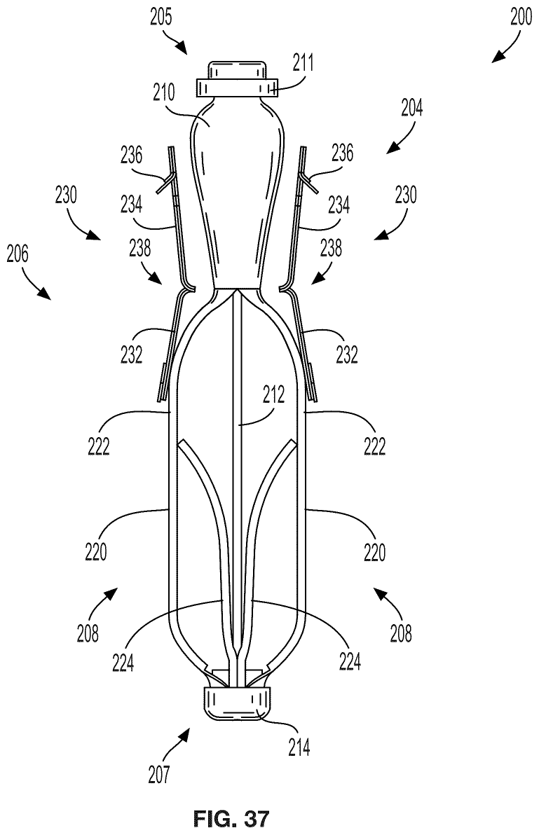

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and movable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the device or implant 200 will become entangled in native anatomy (e.g., chordae tendineae CT shown in FIGS. 3 and 4) when positioning and/or retrieving the device or implant 200 into the delivery system 202.

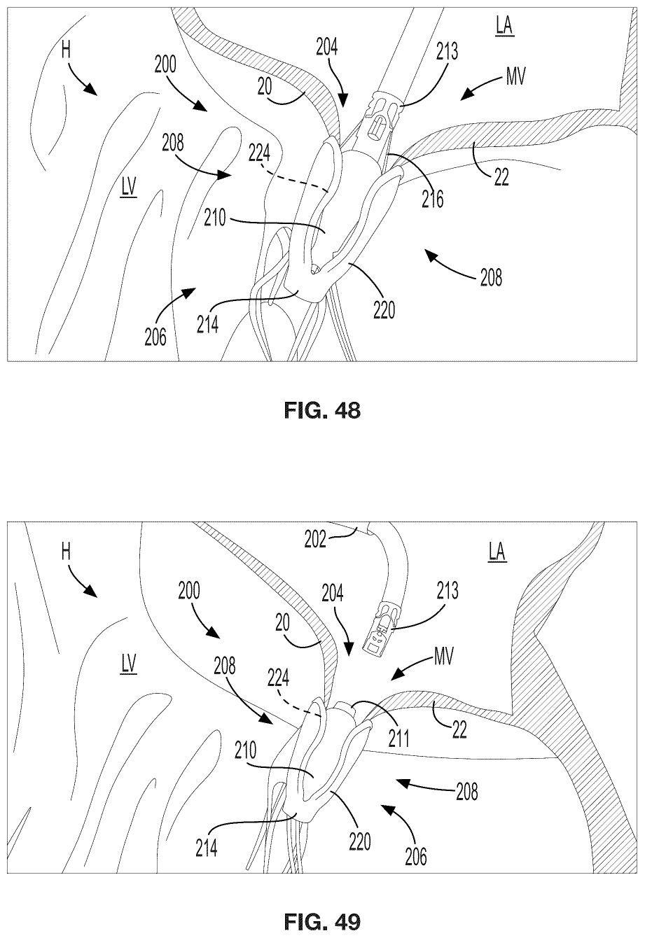

Referring now to FIGS. 38-49, an example device 200 is shown being delivered and deployed within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

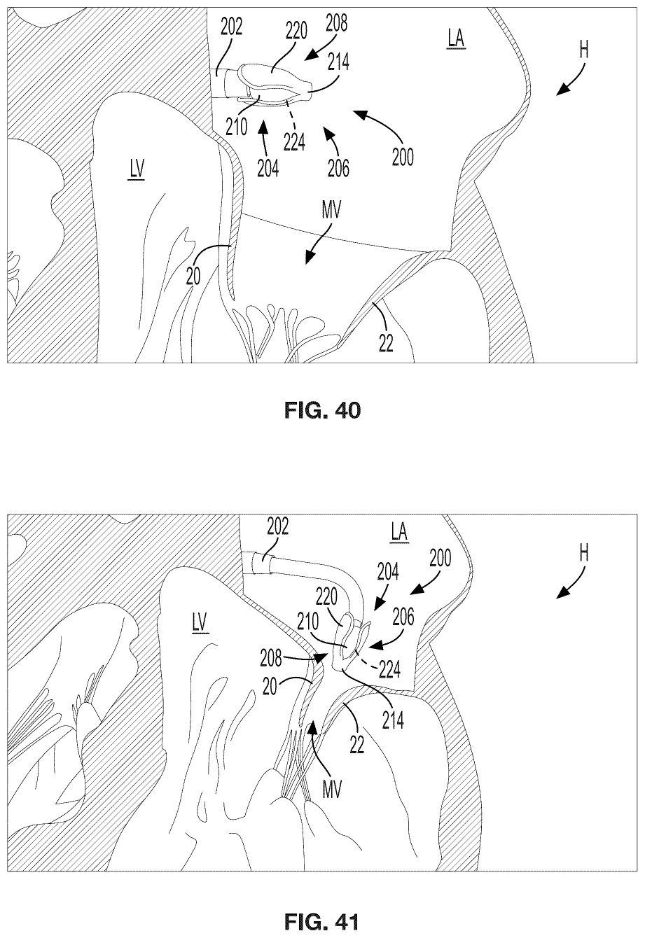

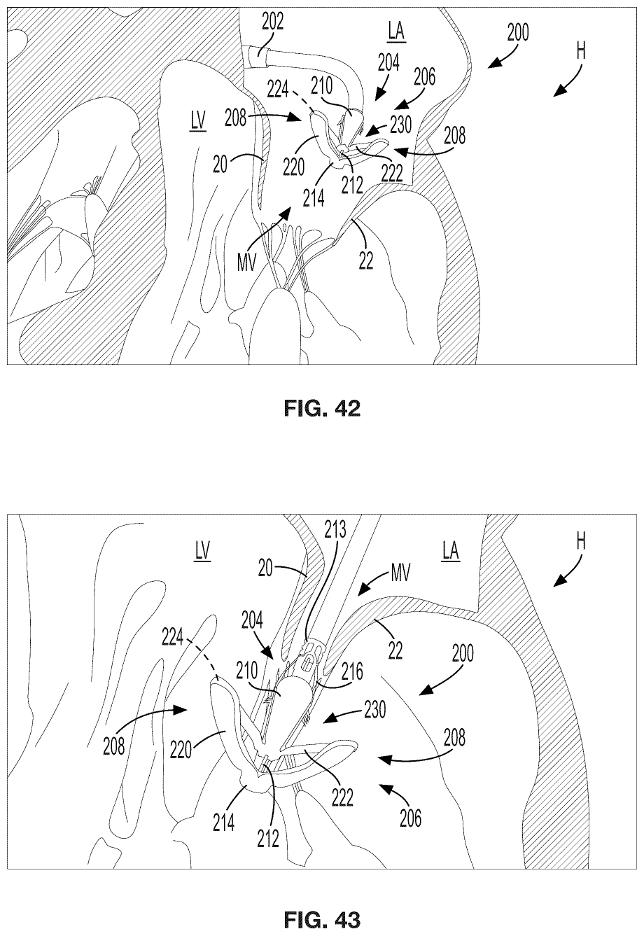

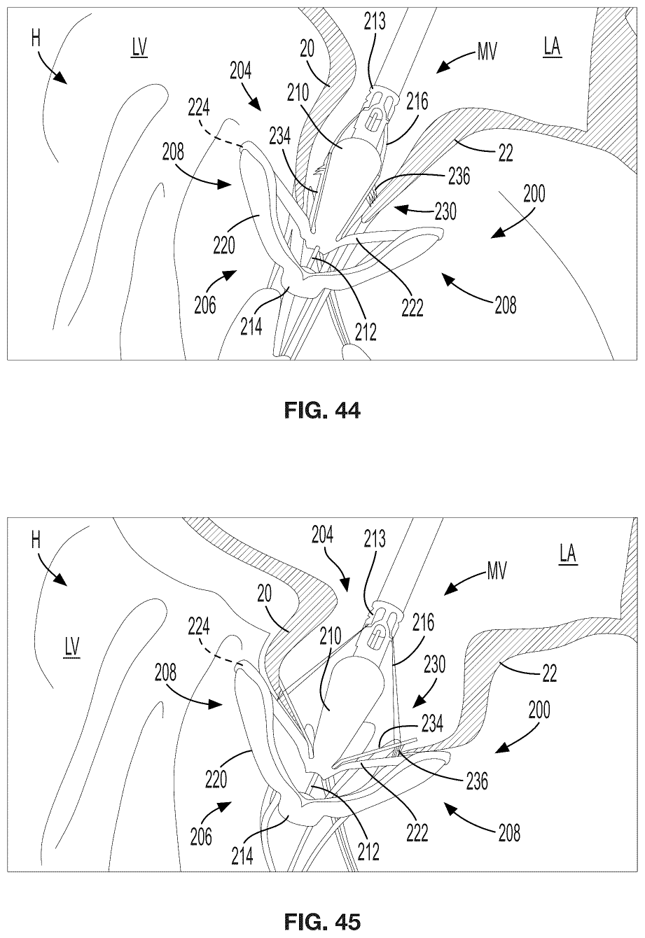

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the optional barbs, friction-enhancing elements, or securing structures 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy—produced in a wire, sheet, tubing, or laser sintered powder—and are biased to hold the outer paddles 220 closed around the inner paddles 222, coaptation element 210, and/or the clasps 230 pinched around native leaflets 20, 22.

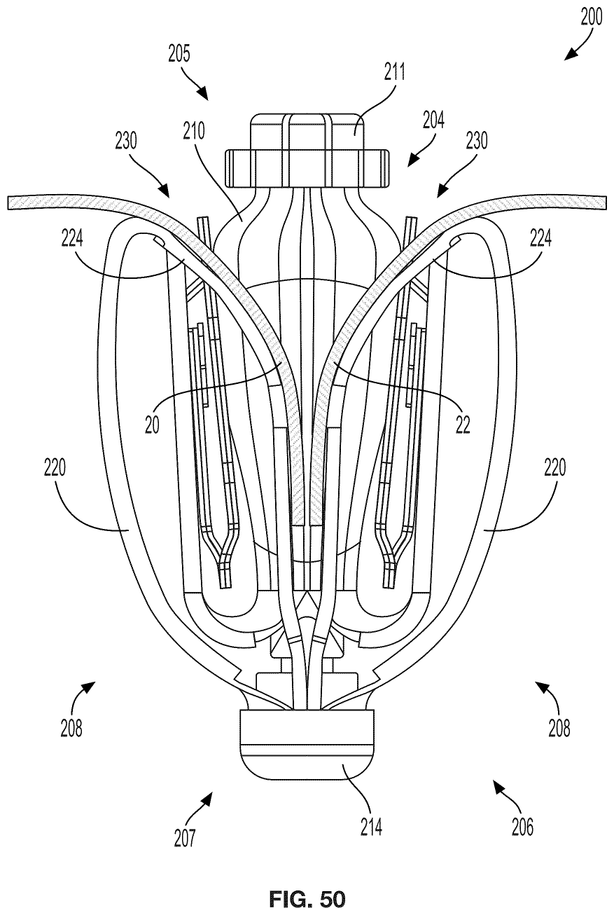

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the mitral valve MV during systole, thereby facilitating repair of functional valve disease that may be causing mitral regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20,22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

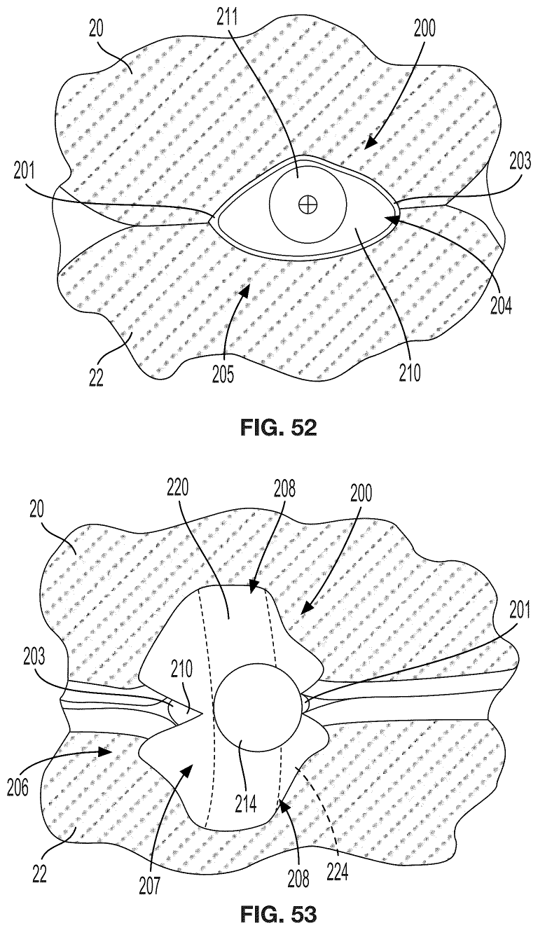

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented or inhibited. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial-lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame 224 shape (toward the valve annulus) can help to achieve coaptation on the lower end of the leaflets, reduce stress, and minimize transvalvular gradients.

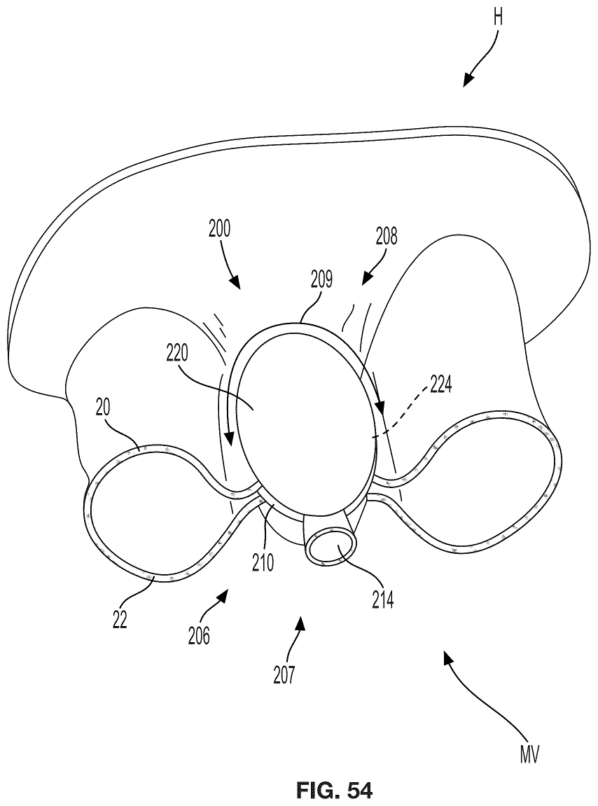

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area 209. For example, in FIG. 54, the force on the leaflets 20, 22 by the paddle frames is spread along the entire rounded length of the paddle frame 224, as the leaflets 20 try to open during the diastole cycle.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and U.S. Provisional Patent App. No. 63/217,622, filed on Jul. 1, 2021. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or U.S. Provisional Patent App. No. 63/217,622. Patent Cooperation Treaty International Application No. PCT/US2018/028189 (International Publication No. WO 2018/195215) and U.S. Provisional Patent App. No. 63/217,622 are incorporated herein by reference in their entirety for all purposes.

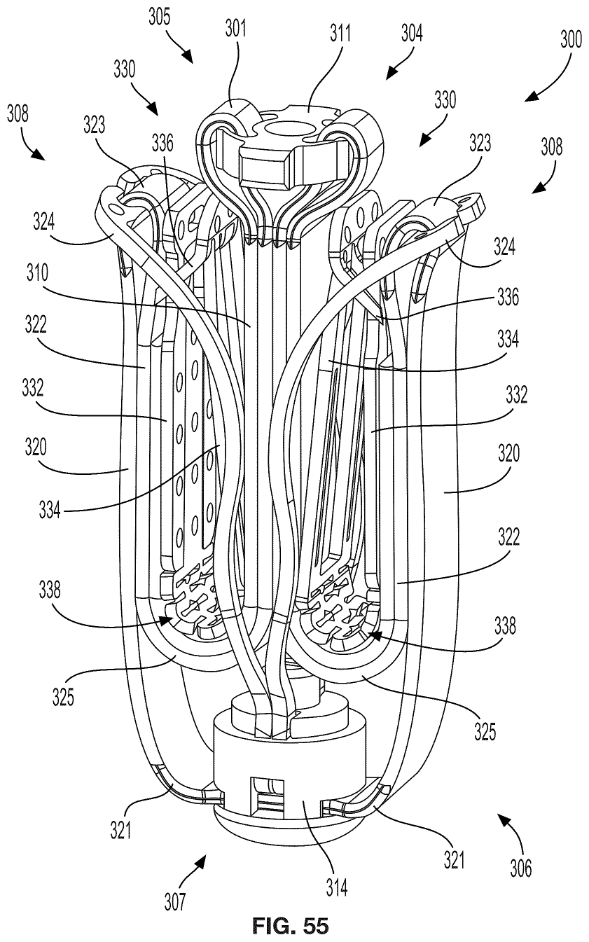

Referring now to FIG. 55, an example of an implantable device or implant 300 (e.g., an implantable prosthetic device, a valve repair device, a valve repair device, etc.) is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion/region 304, and the coaptation portion/region 304 can optionally include a coaptation element 310 (e.g., spacer, plug, membrane, sheet, gap filler, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49 or another capture mechanism described herein or otherwise known) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation member or coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations with a coaptation member or coaptation element 310, the coaptation member or coaptation element 310 and the anchors 308 can be coupled together in various ways. For example, as shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In some implementations, as illustrated, the coaptation element 310, the outer paddle portions 320, the inner paddle portions 322, and the connection portions 321, 323, 325 are formed from a continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device. For example, the anchors 308 can be positioned in a fully extended or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

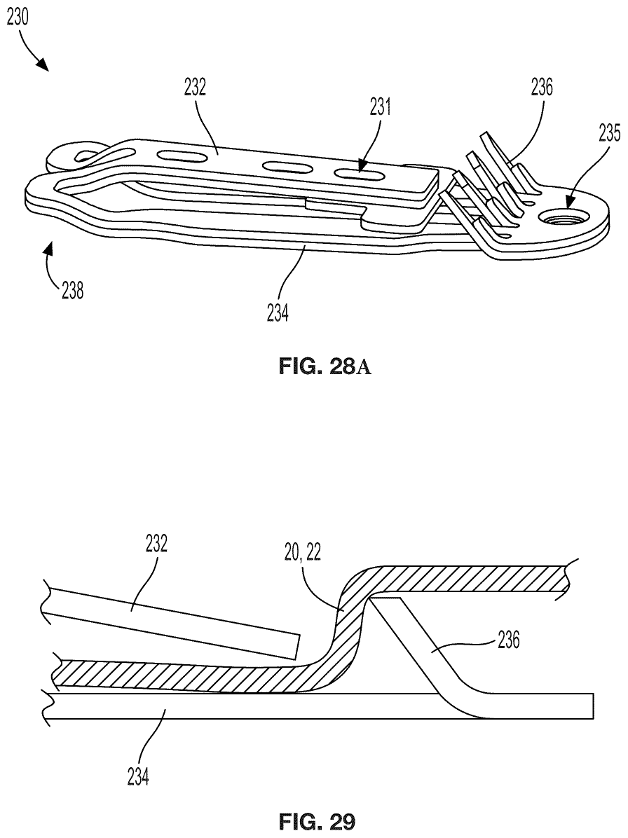

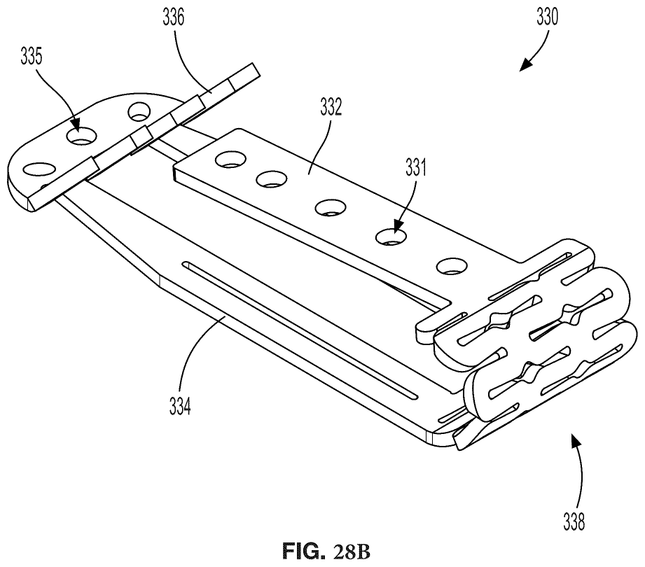

In some implementations, the clasps comprise a movable arm coupled to an anchor. In some implementations, the clasps 330 (as shown in detail in FIG. 28B) include a base or fixed arm 332, a movable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and movable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the movable arms 334 are opened to open the clasps 330 and expose the optional barbs, friction-enhancing elements, or securing structures. The clasps 330 are opened by applying tension to actuation lines (e.g., the actuation lines 216 shown in FIGS. 43-48) attached to holes 335 in the movable arms 334, thereby causing the movable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which can be wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and U.S. Provisional Patent App. No. 63/217,622. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or U.S. Provisional Patent App. No. 63/217,622. Patent Cooperation Treaty International Application No. PCT/US2019/055320 (International Publication No. WO 2020/076898) and U.S. Provisional Patent App. No. 63/217,622 are incorporated herein by reference in their entireties for all purposes.

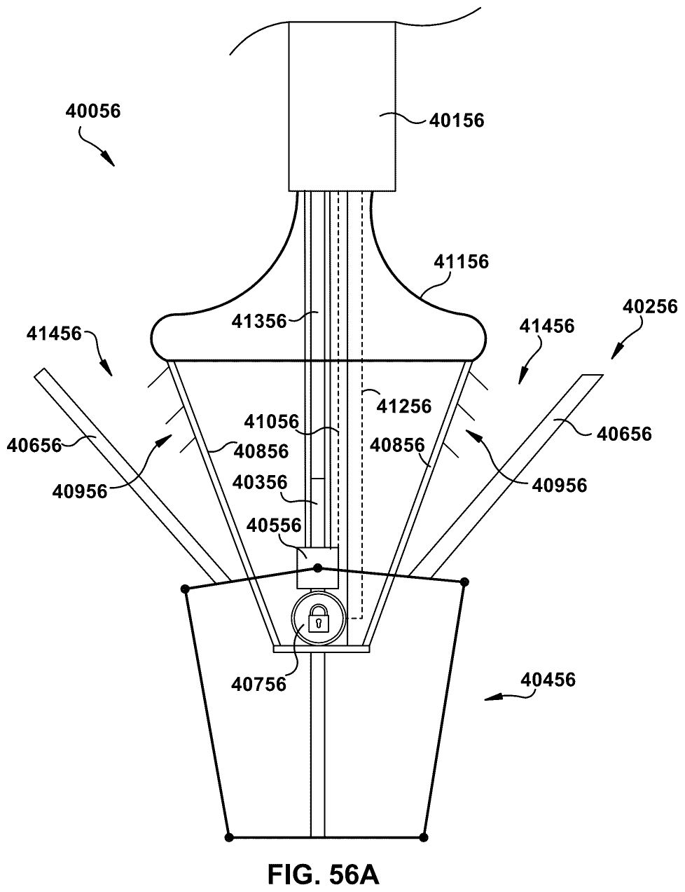

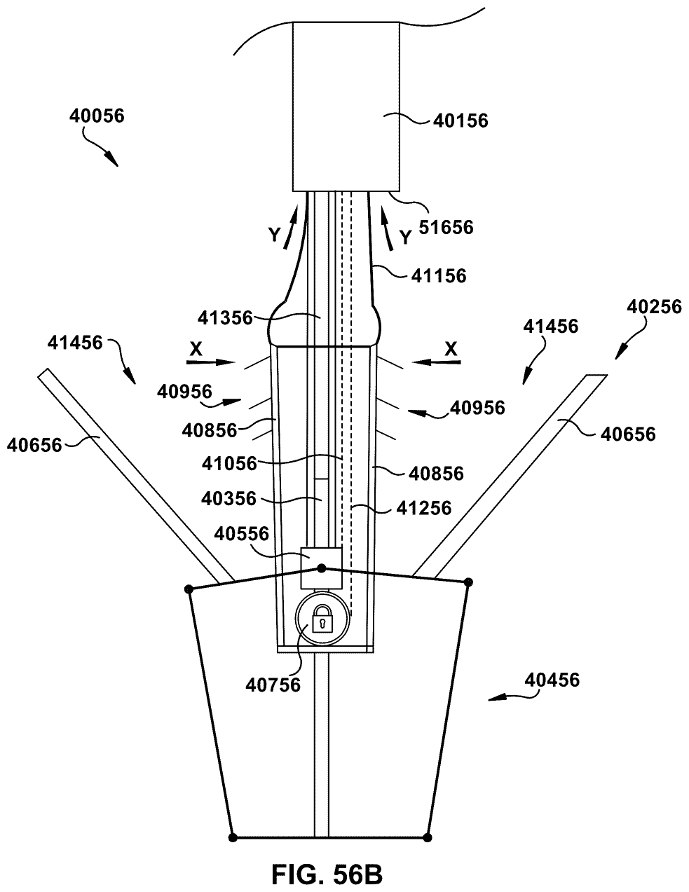

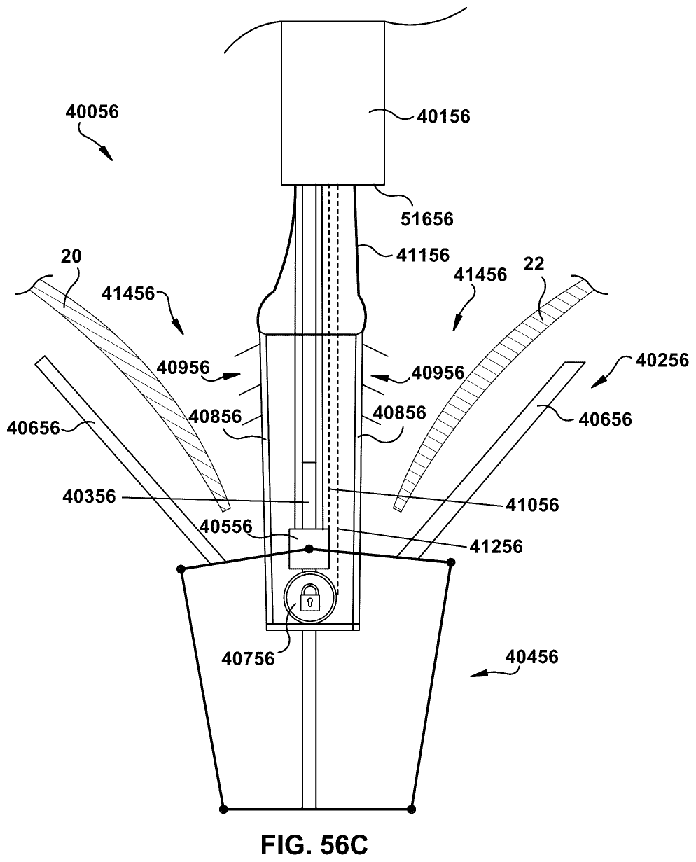

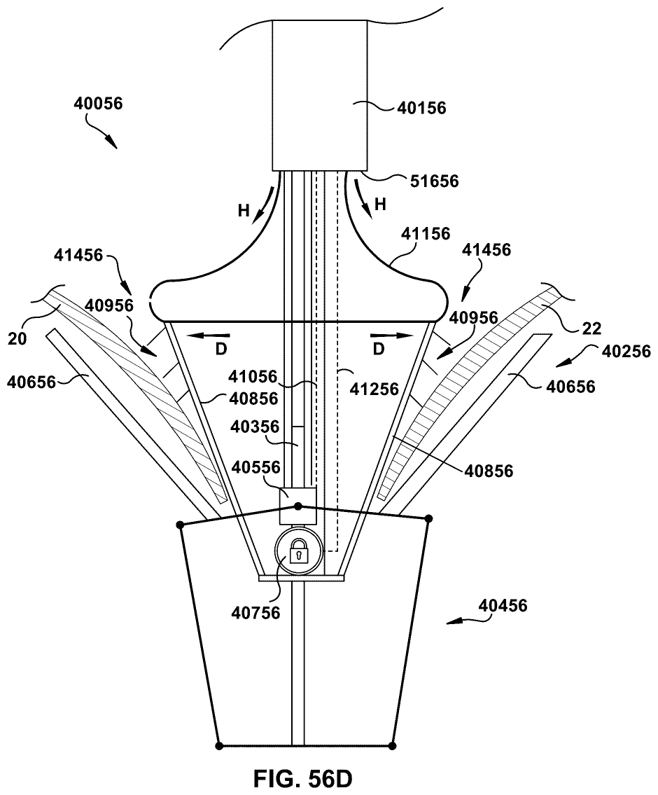

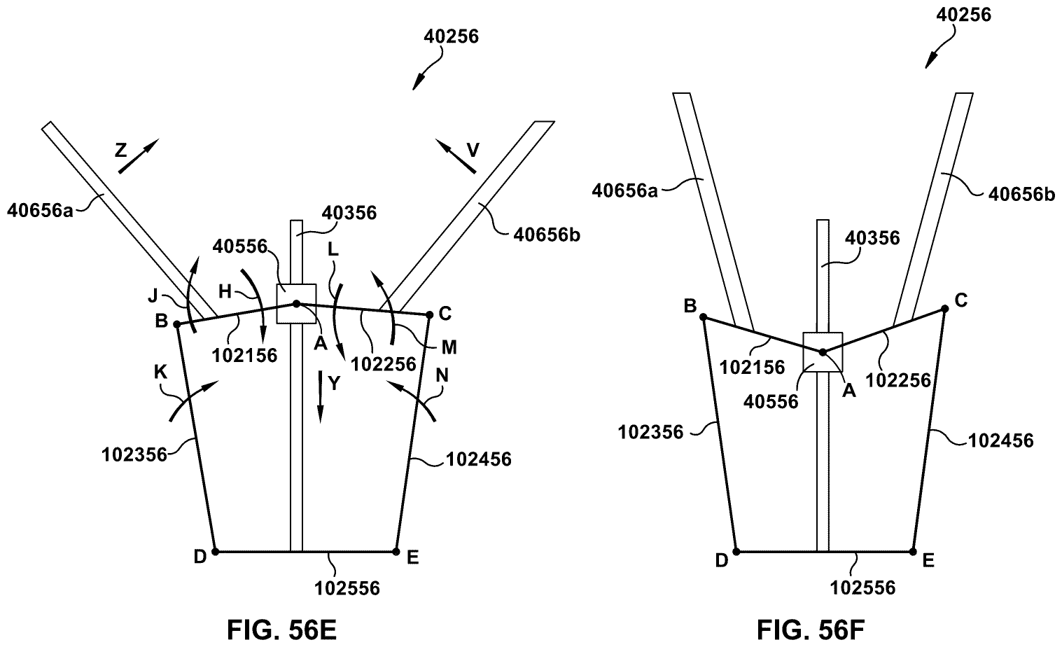

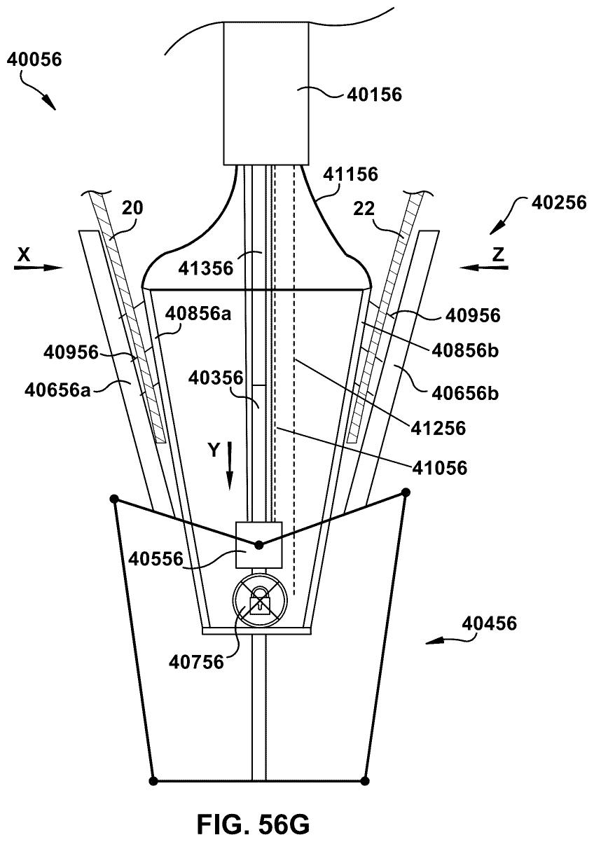

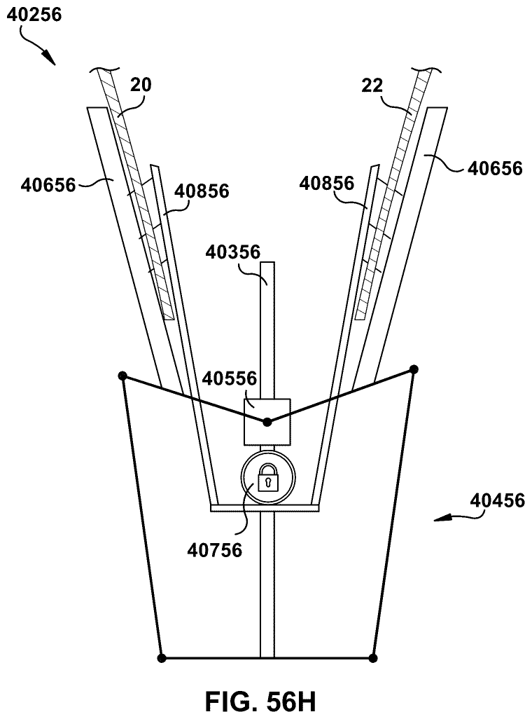

The concepts disclosed by the present application can be used with a wide variety of different valve repair devices. FIGS. 56A-56H illustrate another example of one of the many valve repair systems 40056 for repairing a native valve of a patient that the concepts of the present application can be applied to. The valve repair system 40056 includes a delivery device 40156 and a valve repair device 40256.

The valve repair device 40256 includes a base assembly 40456 and an anchor portion. In some implementations, the anchor portion comprises a pair of paddles 40656 and a pair of gripping members 40856. In some implementations, the paddles 40656 can be integrally formed with the base assembly. For example, the paddles 40656 can be formed as extensions of links of the base assembly. In some implementations, as illustrated, the base assembly 40456 of the valve repair device 40256 has a shaft 40356, a coupler 40556 configured to move along the shaft, and a lock 40756 configured to lock the coupler in a stationary position on the shaft. The coupler 40556 is mechanically connected to the paddles 40656, such that movement of the coupler 40556 along the shaft 40356 causes the paddles to move between an open position and a closed position. In this way, the coupler 40556 serves as a means for mechanically coupling the paddles 40656 to the shaft 40356 and, when moving along the shaft 40356, for causing the paddles 40656 to move between their open and closed positions.

In some implementations, the gripping members 40856 are pivotally connected to the base assembly 40456 (e.g., the gripping members 40856 can be pivotally connected to the shaft 40356, or any other suitable member of the base assembly), such that the gripping members can be moved to adjust the width of the opening 41456 between the paddles 40656 and the gripping members 40856. The gripping member 40856 can include a gripping portion (e.g., barbs, protrusions, ridges, grooves, textured surfaces, adhesive, etc.) 40956 for attaching the gripping members to valve tissue when the valve repair device 40256 is attached to the valve tissue. The gripping member 40856 forms a means for gripping the valve tissue (in particular tissue of the valve leaflets) with a sticking means or portion such as the barbed portion 40956. When the paddles 40656 are in the closed position, the paddles engage the gripping members 40856, such that, when valve tissue is attached to the barbed portion 40956 of the gripping members, the paddles act as holding or securing means to hold the valve tissue at the gripping members and to secure the valve repair device 40256 to the valve tissue. In some implementations, the gripping members 40856 are configured to engage the paddles 40656 such that the barbed portion 40956 engages the valve tissue member and the paddles 40656 to secure the valve repair device 40256 to the valve tissue member. For example, in certain situations, it can be advantageous to have the paddles 40656 maintain an open position and have the gripping members 40856 move outward toward the paddles 40656 to engage valve tissue and the paddles 40656.

While the examples shown in FIGS. 56A-56H illustrate a pair of paddles 40656 and a pair of gripping members 40856, it should be understood that the valve repair device 40256 can include any suitable number of paddles and gripping members.

In some implementations, the valve repair system 40056 includes a placement shaft 41356 that is removably attached to the shaft 40356 of the base assembly 40456 of the valve repair device 40256. After the valve repair device 40256 is secured to valve tissue, the placement shaft 41356 is removed from the shaft 40356 to remove the valve repair device 40256 from the remainder of the valve repair system 40056, such that the valve repair device 40256 can remain attached to the valve tissue, and the delivery device 40156 can be removed from a patient's body.

The valve repair system 40056 can also include a paddle control mechanism 41056, a gripper control mechanism 41156, and a lock control mechanism 41256. The paddle control mechanism 41056 is mechanically attached to the coupler 40556 to move the coupler along the shaft, which causes the paddles 40656 to move between the open and closed positions. The paddle control mechanism 41056 can take any suitable form, and can comprise, for example, a shaft, wire tube, hypotube, rod, suture, line, etc. For example, the paddle control mechanism can comprise a hollow shaft, a catheter tube or a sleeve that fits over the placement shaft 41356 and the shaft 40356 and is connected to the coupler 40556.

The gripper control mechanism 41156 is configured to move the gripping members 40856 such that the width of the opening 41456 between the gripping members and the paddles 40656 can be altered. The gripper control mechanism 41156 can take any suitable form, such as, for example, a line, a suture, a wire, a rod, a catheter, a tube, a hypotube, etc.

The lock control mechanism 41256 is configured to lock and unlock the lock. The lock 40756 serves as a locking means for locking the coupler 40556 in a stationary position with respect to the shaft 40356 and can take a wide variety of different forms and the type of lock control mechanism 41256 can be dictated by the type of lock used. In some implementations, the lock 40756 includes a pivotable plate having a hole, in which the shaft 40356 of the valve repair device 40256 is disposed within the hole of the pivotable plate. In this implementation, when the pivotable plate is in the tilted position, the pivotable plate engages the shaft 40356 to maintain a position on the shaft 40356, but, when the pivotable plate is in a substantially non-tilted position, the pivotable plate can be moved along the shaft (which allows the coupler 40556 to move along the shaft 40356). In other words, the coupler 40556 is prevented or inhibited from moving in the direction Y (as shown in FIG. 56E) along the shaft 40356 when the pivotable plate of the lock 40756 is in a tilted (or locked) position, and the coupler is allowed to move in the direction Y along the shaft 40356 when the pivotable plate is in a substantially non-tilted (or unlocked) position. In implementations in which the lock 40756 includes a pivotable plate, the lock control mechanism 41256 is configured to engage the pivotable plate to move the plate between the tilted and substantially non-tilted positions. The lock control mechanism 41256 can be, for example, a rod, a suture, a wire, or any other member that is capable of moving a pivotable plate of the lock 40756 between a tilted and substantially non-tilted position. In some implementations, the pivotable plate of the lock 40756 is biased in the tilted (or locked) position, and the lock control mechanism 41256 is used to move the plate from the tilted position to the substantially non-tilted (or unlocked) position. In some implementations, the pivotable plate of the lock 40756 is biased in the substantially non-tilted (or unlocked) position, and the lock control mechanism 41256 is used to move the plate from the substantially non-tilted position to the tilted (or locked) position.

FIGS. 56E-56F illustrate the valve repair device 40256 moving from an open position (as shown in FIG. 56E) to a closed position (as shown in FIG. 56F). The base assembly 40456 includes a first link 102156 extending from point A to point B, a second link 102256 extending from point A to point C, a third link 102356 extending from point B to point D, a fourth link 102456 extending from point C to point E, and a fifth link 102556 extending from point D to point E. The coupler 40556 is movably attached to the shaft 40356, and the shaft 40356 is fixed to the fifth link 102556. The first link 102156 and the second link 102256 are pivotally attached to the coupler 40556 at point A, such that movement of the coupler 40556 along the shaft 40356 moves the location of point A and, consequently, moves the first link 102156 and the second link 102256. The first link 102156 and the third link 102356 are pivotally attached to each other at point B, and the second link 102256 and the fourth link 102456 are pivotally attached to each other at point C. One paddle 40656a is attached to first link 102156 such that movement of first link 102156 causes the paddle 40656a to move, and the other paddle 40656b is attached to the second link 102256 such that movement of the second link 102256 causes the paddle 40656b to move. In some implementations, the paddles 40656a, 40656b can be connected to links 102356, 102456 or be extensions of links 102356, 102456.

In order to move the valve repair device from the open position (as shown in FIG. 56E) to the closed position (as shown in FIG. 56F), the coupler 40556 is moved along the shaft 40356 in the direction Y, which moves the pivot point A for the first link 102156 and the second link 102256 to a new position. Movement of the coupler 40556 (and pivot point A) in the direction Y causes a portion of the first link 102156 near point A to move in the direction H, and the portion of the first link 102156 near point B to move in the direction J. The paddle 40656a is attached to the first link 102156 such that movement of the coupler 40556 in the direction Y causes the paddle 40656a to move in the direction Z. In addition, the third link 102356 is pivotally attached to the first link 102156 at point B such that movement of the coupler 40556 in the direction Y causes the third link 102356 to move in the direction K. Similarly, movement of the coupler 40556 (and pivot point A) in the direction Y causes a portion of the second link 102256 near point A to move in the direction L, and the portion of the second link 102256 near point C to move in the direction M. The paddle 40656b is attached to the second link 102256 such that movement of the coupler 40556 in the direction Y causes the paddle 40656b to move in the direction V. In addition, the fourth link 102456 is pivotally attached to the second link 102256 at point C such that movement of the coupler 40556 in the direction Y causes the fourth link 102456 to move in the direction N. FIG. 56F illustrates the final position of the valve repair device 40256 after the coupler 40556 is moved as shown in FIG. 56E.

Referring to FIG. 56B, the valve repair device 40256 is shown in the open position (similar to the position shown in FIG. 56E), and the gripper control mechanism 41156 is shown moving the gripping members 40856 to provide a wider gap at the opening 41456 between the gripping members and the paddles 40656. In some implementations, as illustrated, the gripper control mechanism 41156 includes a line, such as a suture, a wire, etc. that is threaded through an opening in an end of the gripping members 40856. Both ends of the line extend through the delivery opening 51656 of the delivery device 40156. When the line is pulled through the delivery opening 51656 in the direction Y, the gripping members 40856 move inward in the direction X, which causes the opening 41456 between the gripping members and the paddles 40656 to become wider.

Referring to FIG. 56C, the valve repair device 40256 is shown such that valve tissue 20, 22 is disposed in the opening 41456 between the gripping members 40856 and the paddles 40656. Referring to FIG. 56D, after the valve tissue 20, 22 is disposed between the gripping members 40856 and the paddles 40656, the gripper control mechanism 41156 is used to lessen the width of the opening 41456 between the gripping members and the paddles. That is, in the illustrated example, the line of the gripper control mechanism 41156 is released from or pushed out of the opening 51656 of the delivery member in the direction H, which allows the gripping members 40856 to move in the direction D to lessen the width of the opening 41456. While the gripper control mechanism 41156 is shown moving the gripping members 40856 to increase the width of the opening 41456 between the gripping members and the paddles 40656 (FIG. 56C), it should be understood that the gripping members may not need to be moved in order to position valve tissue in the opening 41456. In certain circumstances, however, the opening 41456 between the paddles 40656 and the gripping members 40856 may need to be wider in order to receive the valve tissue.

Referring to FIG. 56G, the valve repair device 40256 is in the closed position and secured to valve tissue 20, 22. The valve repair device 40256 is secured to the valve tissue 20 by the paddles 40656a, 40656b and the gripping members 40856a, 40856b. In particular, the valve tissue 20,22 is attached to the valve repair device 40256 by the gripping portion 40956 of the gripping members 40856a, 40856b, and the paddles 40656a, 40656b engage the gripping members 40856 to secure the valve repair device 40256 to the valve tissue 20, 22.

In order to move the valve repair device 40256 from the open position to the closed position, the lock 40756 is moved to an unlocked condition (as shown in FIG. 56G) by the lock control mechanism 41256. Once the lock 40756 is in the unlocked condition, the coupler 40556 can be moved along the shaft 40356 by the paddle control mechanism 41056. In some implementations, as illustrated, the paddle control mechanism 41056 moves the coupler 40556 in a direction Y along the shaft, which causes one paddle 40656a to move in a direction X and the other paddle 40656b to move in a direction Z. The movement of the paddles 40656a, 40656b in the direction X and the direction Z, causes the paddles to engage the gripping members 40856a, 40856b and secure the valve repair device 40256 to the valve tissue 20, 22.

Referring to FIG. 56H, after the paddles 40656 are moved to the closed position to secure the valve repair device 40256 to the valve tissue 20, 22 (as shown in FIG. 56G), the lock 40756 is moved to the locked condition by the lock control mechanism 41256 (FIG. 56G) to maintain the valve repair device 40256 in the closed position. After the valve repair device 40256 is maintained in the locked condition by the lock 40756, the valve repair device 40256 is removed from the delivery device 40156 by disconnecting the shaft 40356 from the placement shaft 41356 (FIG. 56G). In addition, the valve repair device 40256 is disengaged from the paddle control mechanism 41056 (FIG. 56G), the gripper control mechanism 41156 (FIG. 56G), and the lock control mechanism 41256. Removal of the valve repair device 40256 from the delivery device 40156 allows the valve repair device to remain secured to valve tissue 20, 22 while the delivery device 40156 is removed from a patient.

Additional features of the device 40256, modified versions of the device, delivery systems for the device, and methods for using the device and delivery system are disclosed by Patent Cooperation Treaty International Application No. PCT/US2019/012707 (International Publication No. WO 2019139904) and U.S. Provisional Patent App. No. 63/217,622. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or U.S. Provisional Patent App. No. 63/217,622. Patent Cooperation Treaty International Application No. PCT/US2019/012707 (International Publication No. WO 2019139904) and U.S. Provisional Patent App. No. 63/217,622 are incorporated herein by reference in their entirety for all purposes.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

During implantation of an implantable device or implant in the native heart valve, movement of the device to the implanted position may be impeded or obstructed by the native heart structures. For example, articulable portions of an implantable device or implant (such as paddle portions of anchors used to secure the device to the native heart valve tissue) may rub against, become temporarily caught, or be temporarily blocked by the chordae tendineae CT (shown in FIGS. 3 and 4) that extend to the valve leaflets. An example implantable device or implant can be configured to reduce the likelihood of the device or implant getting temporarily caught or blocked by the CT. For example, the implantable device or implant can take a wide variety of different configurations that are configured to be actively or passively narrowed to reduce the width of a paddle frame of an anchor portion of the device and, consequently, reduce the surface area of the device, which will make it easier to move the device/implant past and/or through the CT.

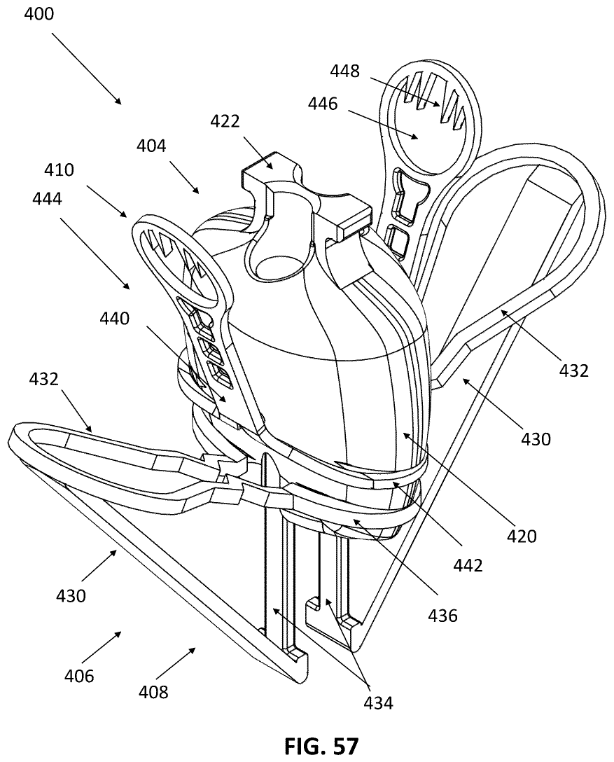

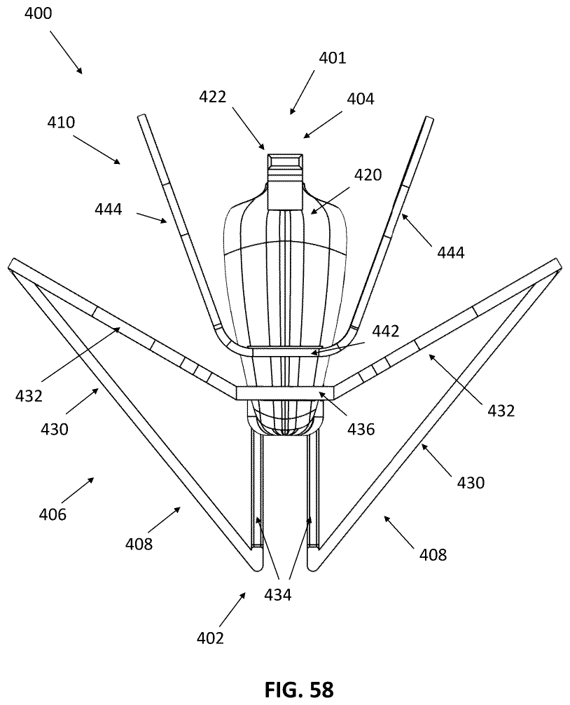

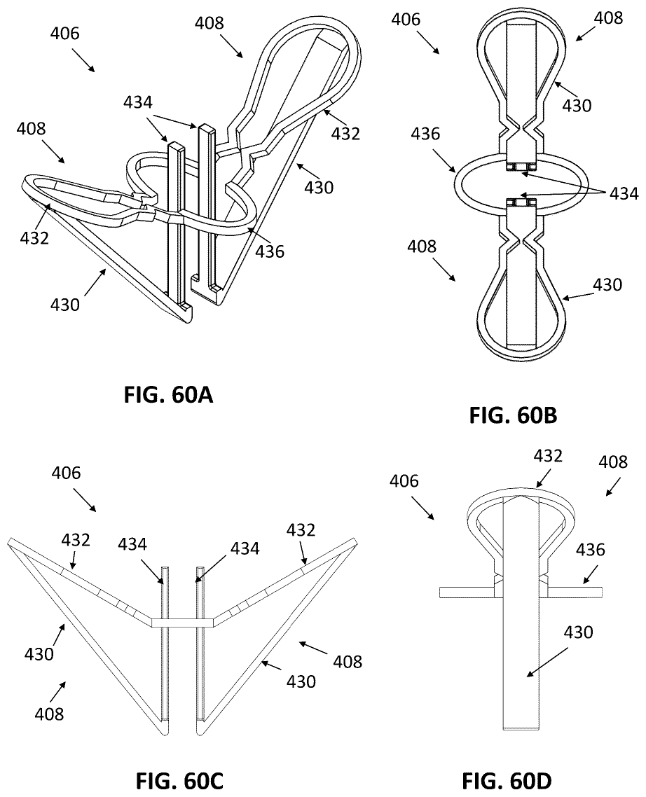

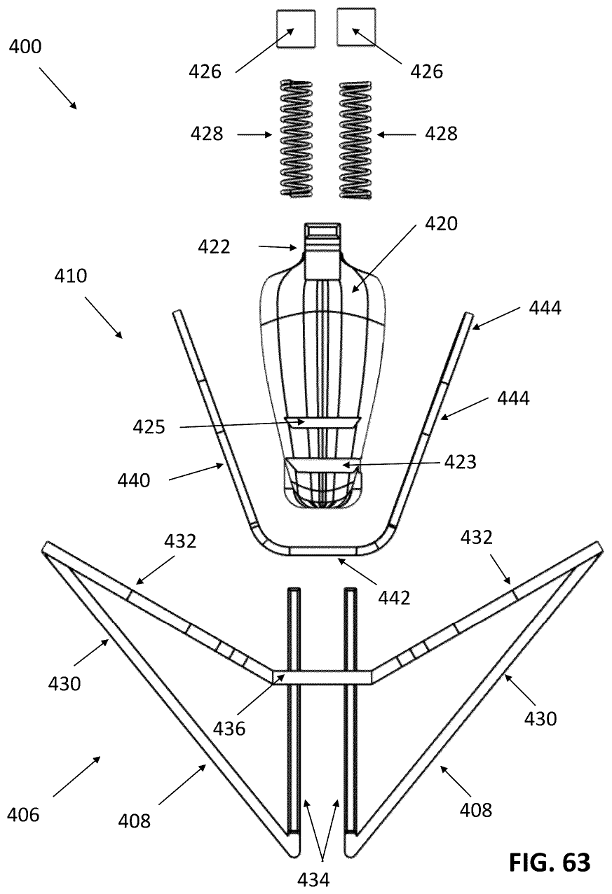

Referring now to FIGS. 57 through 67, an example of a device 400 (e.g., an implantable prosthetic device, a prosthetic spacer device, a valve repair device, etc.) is shown. The valve repair device 400 is one of the many different configurations that the device 100 that is schematically illustrated in FIGS. 8-15 can take. The device 400 can include any other features for an implantable prosthetic device discussed in the present application or any of the applications that are incorporated herein by reference, and the device 400 can be positioned to engage valve tissue 20, 22 as part of any suitable valve repair system (e.g., any valve repair system disclosed in the present application or any of the applications that are incorporated herein by reference). The various components of the valve repair device 400 can be made at any suitable size to accommodate different size patient anatomy.

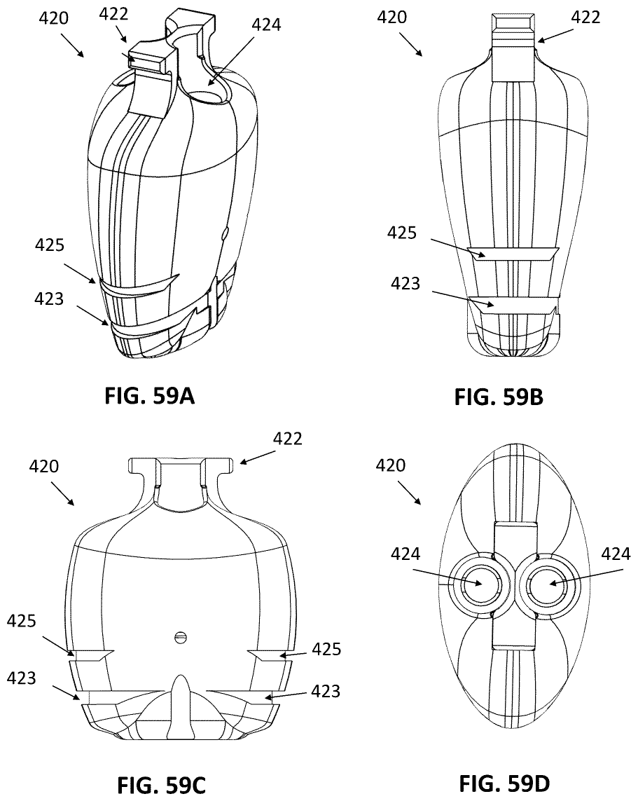

The valve repair device 400 extends from a proximal portion 401 to a distal portion 402 The valve repair device can include an optional coaptation portion 404 and an anchor portion. In some implementations, the anchor portion comprises a paddle portion 406 and/or an attachment portion 410. The coaptation portion 404 (e.g., spacer, coaption element, gap filler, membrane, sheet, plug, wedge, and balloon etc.) includes a coaptation element 420 for implantation between the leaflets 20, 22 of the native valve. The coaptation element 420 has a generally elongated and round shape. In particular, the coaptation element 420 has an elliptical shape or cross-section when viewed from above (FIG. 59D) and has a tapered shape or cross-section when seen from a front view (e.g., FIG. 59C). A blend of these three geometries can result in the three-dimensional shape of the illustrated coaptation element 420 that achieves the benefits described herein. The rounded shape of the coaptation element 420 can also be seen, when viewed from above, to substantially follow or be close to the shape of the collars for the attachment portion 410 and the paddle portion 406, as described below.

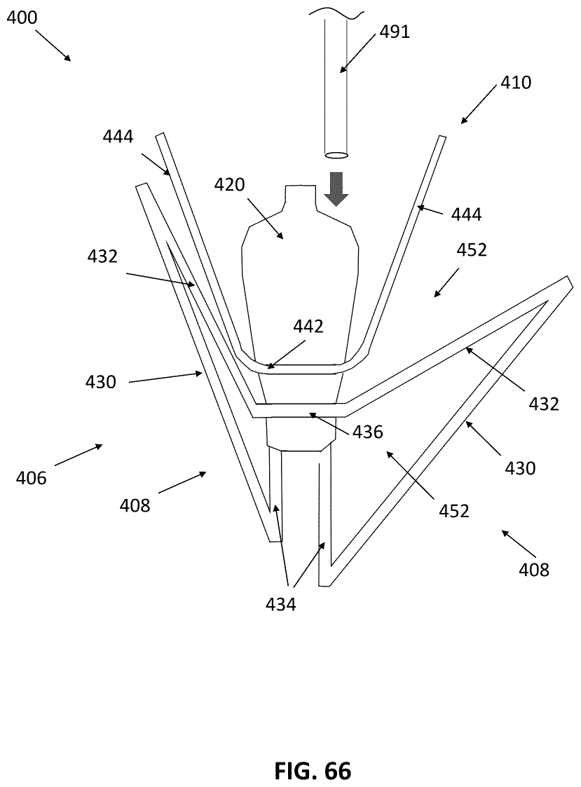

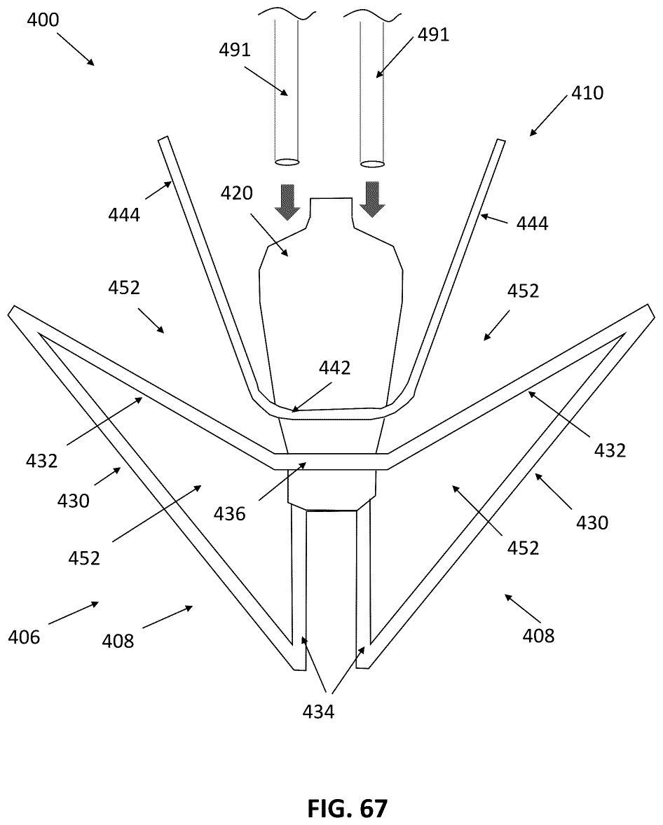

As shown in FIGS. 59A through 59D, the coaptation element 420 (e.g., spacer, coaption element, gap filler, membrane, sheet, plug, wedge, and balloon etc.) can include a boss or connection portion 422 extending upwardly from a proximal portion of the coaptation element 420. The boss or connection portion 422 can be sized and shaped to be secured and/or manipulated by a user during an implantation operation. The boss or connection portion 422 can be sized and shaped such that the coaptation element 420 can be retained, deployed, positioned, recaptured, repositioned, and/or redeployed during the implantation operation. For example, the boss or connection portion 422 can be sized, shaped, or otherwise configured to be engaged or positioned by an actuation element (e.g., actuation shaft, actuation rod, actuation tube, actuation wire, actuation line, etc.) (FIGS. 66-67), and/or to be removably attach to a delivery system or capture mechanism.

The coaptation element 420 can include one or more paddle securing recesses 423 and one or more clasp securing recesses 425 extending inwardly into outer portions of the coaptation element 420. The paddle securing recesses 423 can be sized and shaped to at least partially receive or secure the paddle portion 406 to the coaptation element 420. The clasp securing recesses 425 can be disposed distal to or below the paddle securing recesses 423 and can be sized and shaped to at least partially receive or secure the attachment portion 410 to the coaptation element 420. However, the connection of the coaptation element 420, the attachment portion 410, and the paddle portion 406 can take any configuration. For example, the coaptation element 420 can include a singular paddle securing recess 423 and a single clasp securing recess 425, each extending around the coaptation element 420, or the coaptation element 420 can be integral with the attachment portion 410 and/or the paddle portion 406.

As shown in FIGS. 59A through 59D, the coaptation element 420 can include two or more passages 424 on either side of the boss or connection portion 422. The passages 424 extend longitudinally through the coaptation element 420 from the proximal portion to the distal portion. The passages 424 can be sized and shaped to receive one or more components which can open and close the valve repair device 400.

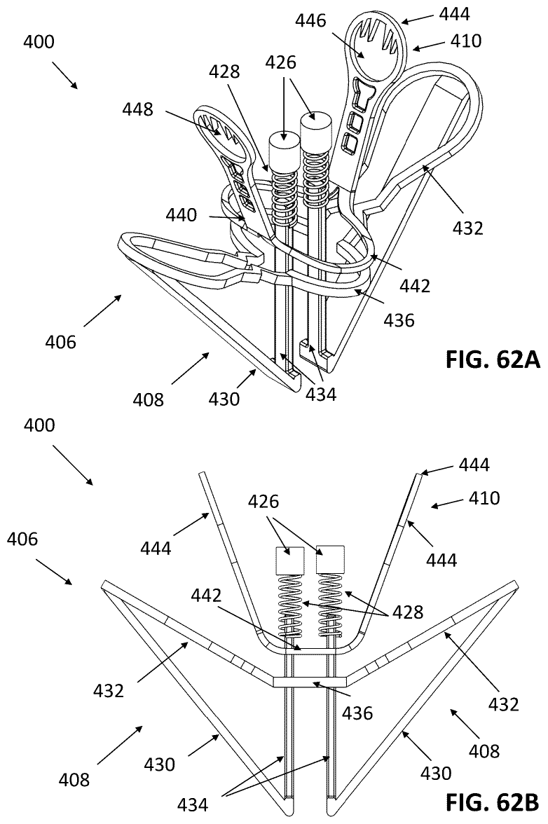

As shown in FIGS. 62A through 64, the coaptation element 420 can include an actuator 426 at least partially disposed in the proximal or upper portion of each passage 424. Each actuator 426 can be sized and shaped to slidably fit in the respective passage 424. The actuators 426 can be a cap, button, or other component which can be actuated by an actuation element (e.g., actuation wire, shaft, rod, line, etc.) extending from a delivery sheath or system (e.g., delivery system 102, 202). For example, the actuators 426 can be configured to be engaged or actuated by an actuation element 491 (e.g., actuation shaft, actuation wire, etc.;

FIGS. 22-24) to at least partially open the valve repair device 400.

The paddle portion 406 of the valve repair device 400 includes a plurality of paddles 408, each paddle 408 including an outer paddle 430, an inner paddle 432, and a paddle extension shaft 434. The paddles 408 can facilitate the engagement of valve tissue 20, 22 as part of any suitable valve repair system. The paddle extension shaft 434 can be a substantially vertical shaft which can be at least partially received in one of the passages 424 of the coaptation element 420.

The outer paddles 430 can be substantially rectangular and can facilitate the engagement of the tissue 20, 22 as part of any suitable valve repair system. However, the outer paddles 430 can also be a wire or mesh frame or any other suitable configuration. Each outer paddle 430 can extend upwardly and outwardly from the distal end of the paddle extension shaft 434. The outer paddle 430 can be flexibly connected to the paddle extension shaft 434 such that the outer paddle 430 can be at least partially pivotable about the end of the paddle extension shaft 434. For example, the outer paddle 430 can be connected to the paddle extension shaft 434 by a hinge, joint, or other pivotable connector or can be flexibly integral with the distal end of the paddle extension shaft 434.

The inner paddles 432 can be generally flat with a bulbous or lightbulb-shaped cross-section with a wider distal end and a narrower medial end. The inner paddles 432 can be a substantially wire frame. However, the inner paddles 432 can be any suitable shape or configuration. For example, the inner paddles 432 can be rectangular or any other shape and can be a mesh or solid frame or any other suitable configuration. Each inner paddle 432 can extend upwardly and radially outwardly from a medial position to the proximal and radially outer end of one of the outer paddles 430. The wider portion of the inner paddle 432 can be flexibly connected to the upper end of the outer paddle 430 such that the outer paddle 430 and/or the inner paddle 432 can be at least partially pivotable about each other. In some implementations, as illustrated, the inner paddle 432 is integral with the outer paddle 430 such that the inner paddle 432 and outer paddle 430 can flex or pivot about each other. However, the inner paddle 432 and outer paddle 430 can be connected by any suitable connection. For example, the inner paddle 432 can be connected to the outer paddle by a hinge, joint, or other pivotable connector.

The paddle portion 406 also includes a rounded or elliptical paddle collar 436 which can connect or secure the paddle portion 406 to the coaptation element 420. For example, the paddle collar 436 can be sized and shaped to fit into the paddle securing recesses 423 and around an outer portion of the coaptation element 420. For example, the paddle collar 436 can be sized and shaped to be at least partially snap fit into the paddle securing recesses 423 of the coaptation element 420. The paddle collar 436 and paddle securing recesses 423 can also be sized, shaped, or otherwise configured such that the paddle collar 436 can be at least partially secured in the paddle securing recesses 423 by an interference fit. Each inner paddle 432 can be connected to the paddle collar 436 and the outer paddle 430 and the outer paddle 430 can be connected to the paddle extension shaft 434 such that each paddle 408 (e.g., outer paddle 430, inner paddle 432, and paddle extension shaft 434) can be articulated, maneuvered, or otherwise articulated independently of the other paddle 408, as described below.

The outer paddles 430, inner paddles 432, paddle extension shafts 434, and paddle collar 436 can be derived from a single super-elastic sheet, ribbon or wire which can resist plastic deformation. In some implementations, as illustrated, the paddle collar 436 is semi-circular and integral with the inner paddles 432. However, the paddle portion 406 can be configured or connected in any suitable manner. For example, the paddle collar 436 can be circular and separate from the inner paddles 432 and the inner paddles 432 can be connected to opposing sides of the paddle collar 436 by a hinge, joint, or other flexible or pivotable connector.

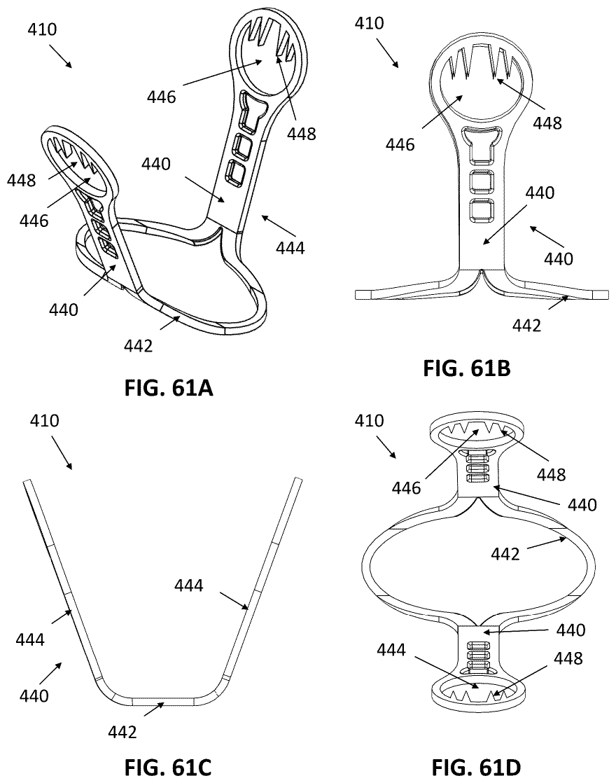

As shown in FIGS. 61A through 61D, attachment portion or gripping members (e.g., gripping arms, clasp arms, etc.) 410 can include a rounded or elliptical collar 442 that connects the two clasp arms together. The collar can connect or secure the attachment portion or gripping members 410 to the coaptation element 420. The collar 442 can be sized and shaped to be secured, placed, or otherwise disposed on the coaptation element 420. For example, the collar 442 can be sized and shaped to be at least partially snap fit into the clasp securing recesses 425 of the coaptation element 420. The collar 442 and clasp securing recesses 425 can also be sized, shaped, or otherwise configured such that the collar 442 can be at least partially secured in the clasp securing recesses 425 by an interference fit.

The attachment portion or gripping members 410 can include two or more clasp elements (clasps, clasp arms, etc.) 444 which can facilitate the engagement of valve tissue 20, 22 as part of any suitable valve repair system. The gripping members 410 can comprise clasps 130 that include a base or fixed arm, a movable arm, optional barbs, friction-enhancing elements, or other means for securing (e.g., protrusions, ridges, grooves, textured surfaces, adhesive, etc.), and a joint portion 138 as illustrated by FIGS. 8-27, 28A, 28B, and 29-51. Each clasp element 444 can have a narrower radially proximal portion 440 which is connected to the collar 442 and a wider radially distal portion which can include a clasp engagement portion 446 for engagement of the valve tissue 20, 22 as part of any suitable valve repair system. In some implementations, as illustrated, the clasp elements 444 are substantially flat with a bulbous or lightbulb or teardrop-shaped cross section. However, the clasp elements 444 can have any suitable size, shape, or configuration. For example, the clasp elements 444 can be substantially solid, a wire frame, rectangular, and/or similar to the size, shape, or configuration of the outer paddles 430 or inner paddles 432.

The clasp elements 444 can extend radially outward and proximally upward from the collar 442. The clasp elements 444 can be integrally formed with the collar 442. The attachment portion or gripping members 410 can be formed from a single, super-elastic sheet, ribbon, or wire such that the attachment portion or gripping members 410 can resist deformation. For example, the attachment portion or gripping members 410 can be formed from a single sheet or piece of material such that the radially outward portions of the clasp elements 444 are urged or biased downwardly or distally. However, the attachment portion or gripping members 410 can have any suitable shape, size, or configuration and the collar 442 and clasp elements 444 can have any suitable connection. For example, the clasp elements 444 can be connected to the collar 442 by a hinge, joint, or other flexible or pivotable connector.

Each clasp element 444 can include one or more projections or barbs 448 extending into the clasp engagement portion 446. The optional barbs 448 can engage the leaflets 20, 22 and secure the valve repair device 400 in the native valve, e.g., native mitral valve, when the device 400 is in the closed position, as described below. The tissue of the leaflet 20, 22 is not pierced by the barbs 448, though in some implementations the barbs 448 can partially or fully pierce through the leaflet 20, 22. In some implementations, as illustrated, the optional barbs 448 extend radially inward into the clasp engagement portion 446 and in line with the remainder of the clasp element 444. However, the barbs 448 can have any suitable size, shape, orientation, or configuration to secure the valve repair device 400 in the native valve. For example, the barbs 448 can be at an angle or perpendicular to the remainder of the clasp element 444 such that the barbs 448 can engage the tissue of the leaflets 20, 22.

As shown in FIGS. 62A through 64, the coaptation element 420 can also include a biasing element 428 in each of the passages 424 which can oppose the force output from the paddle portion 406 when the valve repair device 400 is the closed position, as detailed below. Each biasing element 428 (e.g., spring, resilient band, compressible member, compressible fluid, etc.) is disposed between and connected or affixed to the actuator 426 and a proximal portion of the paddle extension shaft 434. In some implementations, as illustrated, the biasing elements 428 are coil springs. However, the biasing elements 428 can be any device or component which can provide a biasing force. For example, the biasing elements 428 can be leaf springs, shape-memory alloys such as Nitinol, or any other biasing device.

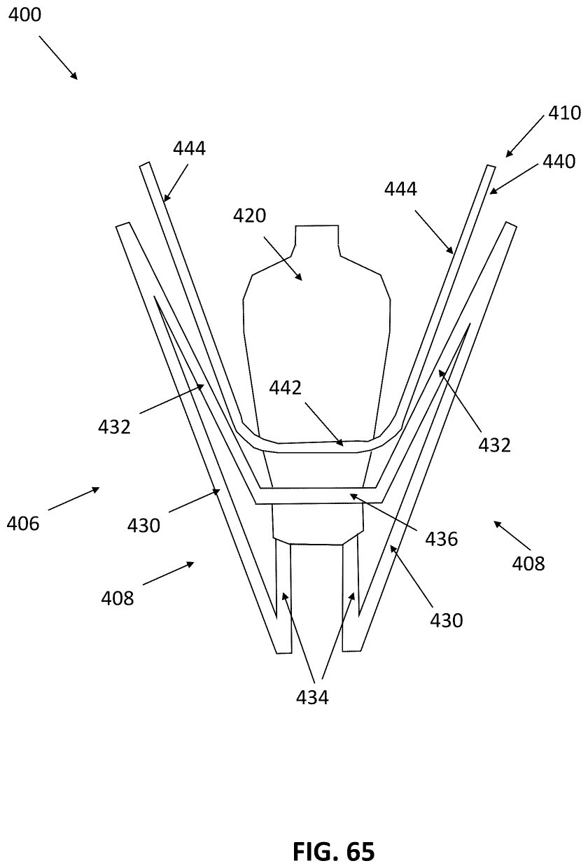

Referring now to FIGS. 65 through 67, the valve repair device 400 can be moved between a closed position and an open position. As shown in FIG. 65, the device 400 can be deployed in the closed position with the paddles 408 (e.g., outer paddles 430, inner paddles 432, and paddle extension shafts 434) pulled proximally upward and radially inward. The biasing elements 428 can keep the paddle extension shafts 434 pulled proximally into the passage 424 of the coaptation element 420. The clasp elements 444 can optionally be pulled proximally upward and radially inward as well. The clasp elements 444 can be in engagement with the inner paddles 432 of the paddles 408.

As shown in FIG. 66, one of the paddles 408 can be moved to the open position with the actuation element (e.g., actuation shaft, actuation rod, actuation tube, actuation wire, actuation line, etc.) 491. The actuation element 491 can engage one of the actuators 426 to oppose the respective biasing element 428 and move the actuator 426 downward. Moving the actuator 426 and/or biasing element 428 distally or downwardly in the passage 424 exerts a downward force on the paddle extension shaft 434 which moves the paddle extension shaft 434 distally or downwardly out of the passage 424. The downward movement of the paddle extension shaft 434 opens the respective paddles 430, 432. The outer paddle 430 is pulled downwardly or distally and the radially outer portion of the outer paddle 432 is pulled radially outward. The movement of the outer paddle 430 pulls the radially outer portion of the inner paddle 432 downwardly and radially outwardly, creating tissue receiving gaps 452 between the outer paddle 430 and inner paddle 432 and between the inner paddle 432 and the clasp element 444.

As shown in FIG. 67, the other paddle 408 can be moved to the open position with one of the actuation elements 491. The same process can be repeated to move the paddle extension shaft 434, the outer paddle 430, and inner paddle 432 to create tissue receiving gaps 452 between the outer paddle 430 and inner paddle 432 and between the inner paddle 432 and the clasp element 444.

While the valve repair device 400 is in the partially open position (e.g., one paddle open in FIG. 66) or the fully open position (e.g., both paddles open in FIG. 67), the valve repair device 400 can be maneuvered, positioned, or otherwise moved to the desired position. The valve repair device 400 can be maneuvered or otherwise moved such that the native leaflets 20, 22 are in one of the tissue receiving gaps 452 on either or both sides of the valve repair device 400. For example, the position or movement of the valve repair device 400 can be controlled by a connection or engagement with the boss or connection portion 422 of the coaptation element 420. For example, the boss or connection portion 422 can be sized, shaped, or otherwise configured to be engaged or positioned by an actuation element 491, and/or to be removably attached to a delivery system, collar, or capture mechanism (e.g., one or more of a clamp, clip, pin, suture, line, lasso, noose, snare, buckle, lock, latch, etc.).

Once one of the paddles 408 is in place in the native valve, e.g., native mitral valve, with the native leaflets 20, 22 in the tissue receiving gaps 452 on either side of the device 400, the respective paddle 408 can be closed. For example, the valve repair device 400 can be closed to capture the native leaflets 20, 22, such as between the inner paddle 432 and the clasp element 444. The actuation element 491 can be retracted proximally or upwardly to disengage the actuator 426. Disengaging the actuator 426 causes the biasing element 428 and paddle extension shaft 434 to retract proximally or upwardly into the passage 424. The upward movement of the paddle extension shaft 434 allows the outer paddle 430 and inner paddle 432 to move upwardly and to flex or pivot inwardly toward the clasp element 444. The outer paddle 430 can move upwardly and inwardly to move the inner paddle 432 upwardly and inwardly to press valve tissue against the clasp element 444. The native leaflets 20, 22 can be secured by the biasing force of the biasing element 428 that acts on the paddles 430, 432 and/or by the distal or downward bias of the clasp element 444.

Once the native leaflets 20, 22 are secured on one side of the valve repair device 400, the valve repair device 400 can be positioned or repositioned such that the native leaflets 20, 22 are disposed in one of the tissue receiving gaps 452 on the other side of the valve repair device 400. The closing process can be repeated for the other paddle 408 when the native leaflets 20, 22 are in place on the other side of the valve repair device 400, such as in one of the tissue receiving gaps 452.

While the process has been described as opening both paddles 408 and closing each paddle 408 in turn, the device 400 can be opened, positioned, and closed in other manners. For example, one paddle 408 can be opened, positioned, and closed in place and then the other paddle 408 can be opened, positioned, and closed in place or both paddles 408 can be opened and positioned and then simultaneously closed in place.

Once the valve repair device 400 is closed in the desired position, the valve repair device 400 can be released from the delivery system or capture mechanism, and actuation element 491 can be withdrawn and removed. The native leaflets 20, 22 can be secured or engaged by the clasp engagement portion 446, such as by the barbs 448 of the clasp elements 444.

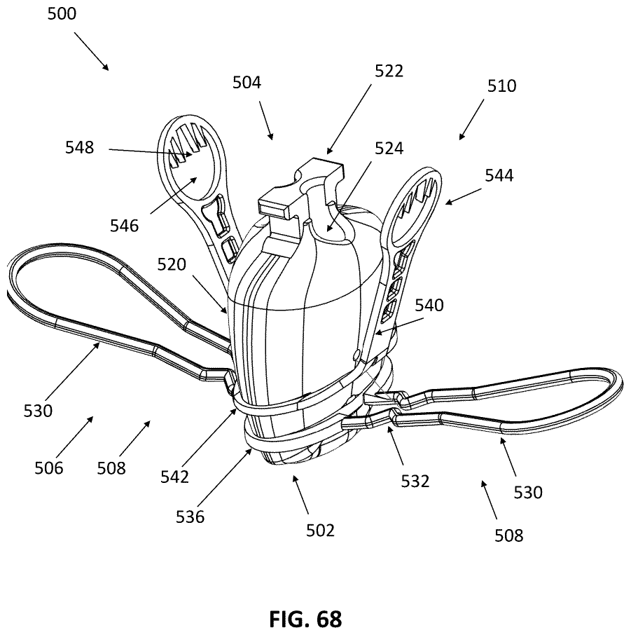

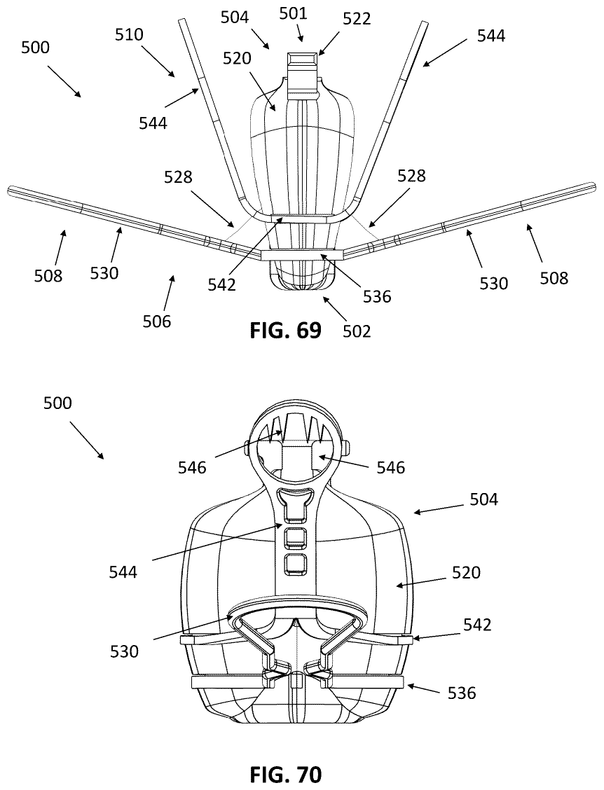

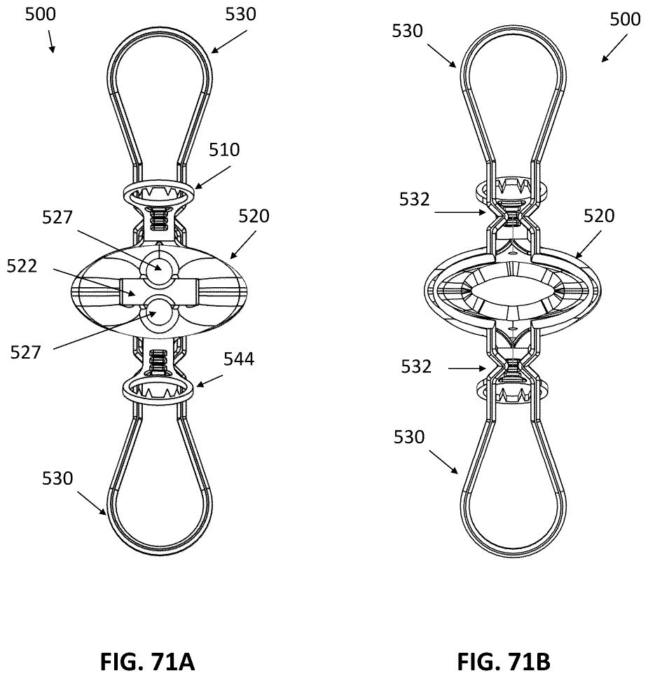

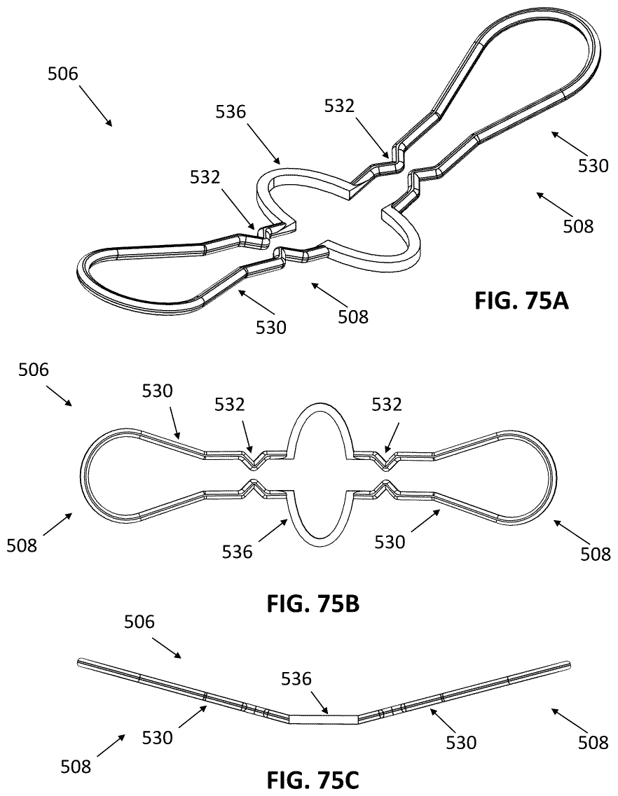

Referring now to FIGS. 68 through 79, an example of a device (e.g., an implantable prosthetic device, a prosthetic spacer device, a valve repair device, etc.) 500 is shown. The valve repair device 500 is one of the many different configurations that the device 100 that is schematically illustrated in FIGS. 8-15 can take. The device 500 can include any other features for an implantable prosthetic device discussed in the present application or any of the applications that are incorporated herein by reference, and the device 500 can be positioned to engage valve tissue 20, 22 as part of any suitable valve repair system (e.g., any valve repair system disclosed in the present application or any of the applications that are incorporated herein by reference). The various components of the valve repair device 500 can be made at any suitable size to accommodate different size patient anatomy.

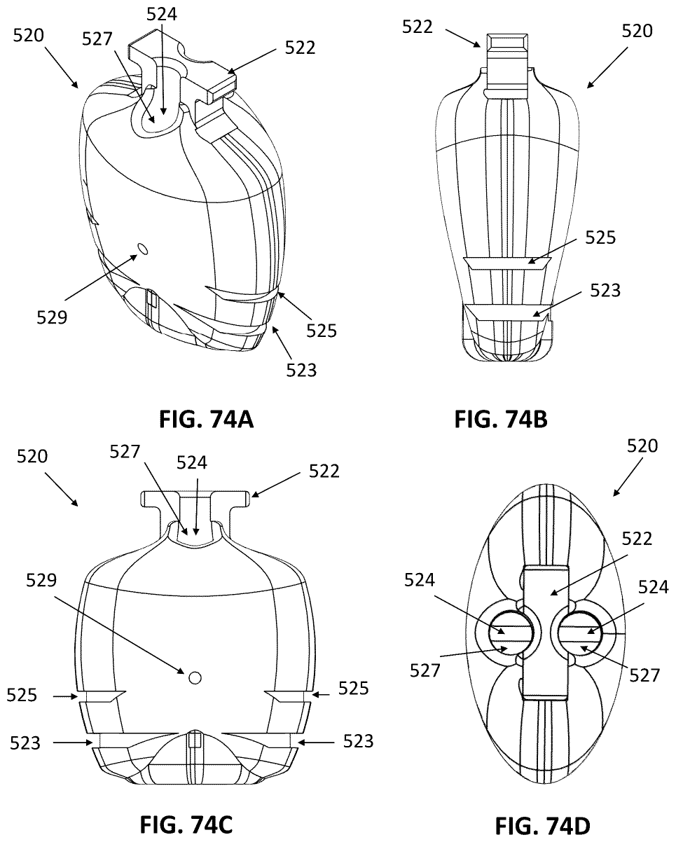

The valve repair device 500 extends from a proximal portion 501 to a distal portion 502 and can include an optional coaptation portion 504 and anchor portion. In some implementations, the anchor portion can comprise a paddle portion 506 and/or an attachment portion or gripping members 510. The coaptation portion 504 includes a coaptation element 520 (e.g., spacer, coaption element, gap filler, membrane, sheet, plug, wedge, and balloon etc.) for implantation between the leaflets 20, 22 of the native valve. The coaptation element 520 has a generally elongated and round shape. In particular, the coaptation element 520 has an elliptical shape or cross-section when viewed from above (FIG. 74D) and has a tapered shape or cross-section when seen from a front view (e.g., FIG. 74C). A blend of these three geometries can result in the three-dimensional shape of the illustrated coaptation element 520 that achieves the benefits described herein. The rounded shape of the coaptation element 520 can also be seen, when viewed from above, to substantially follow or be close to the shape of the collars for the attachment portion or gripping members and the paddle portion, as described below.

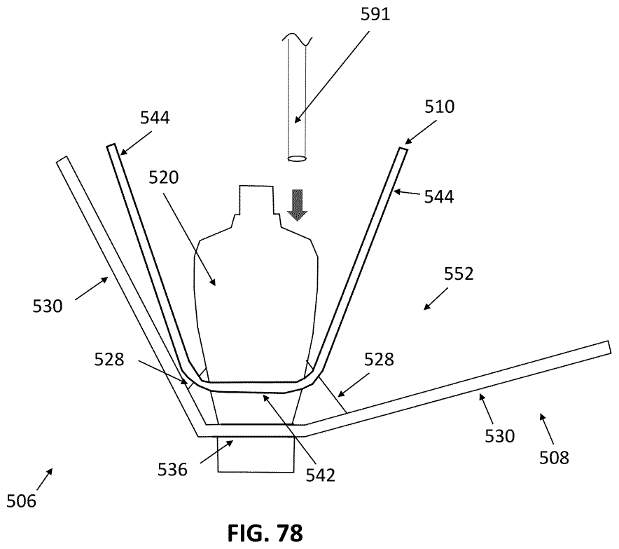

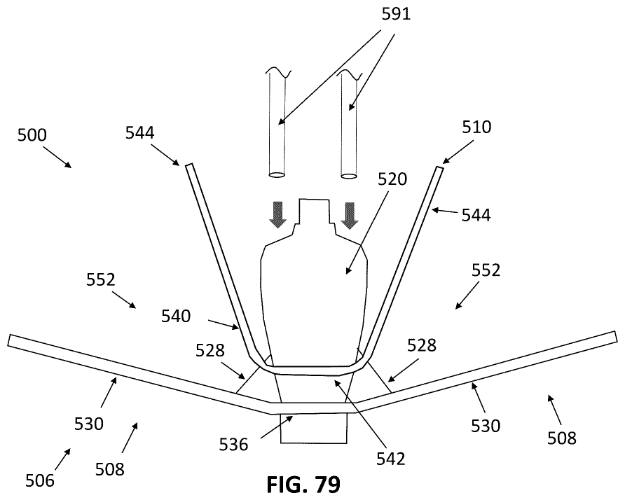

As shown in FIGS. 74A through 74D, the coaptation element 520 can include a boss or connection portion 522 extending upwardly from a proximal portion of the coaptation element 520. The boss or connection portion 522 can be substantially similar to the boss or connection portion 422 of FIGS. 57 through 67. The boss or connection portion 522 can be sized and shaped to be secured and/or manipulated by a user during an implantation operation. The boss or connection portion 522 can be sized and shaped such that the coaptation element 520 can be retained, deployed, positioned, recaptured, repositioned, and/or redeployed during the implantation operation. For example, the boss or connection portion 522 can be sized, shaped, or otherwise configured to be engaged or positioned by an actuation element (e.g., actuation shaft, actuation rod, actuation tube, actuation wire, actuation line, etc.) 591 (FIGS. 78-79), and/or to be removably attach to a delivery system or capture mechanism (e.g. one or more of a clamp, clip, pin, suture, line, lasso, noose, snare, buckle, lock, latch, etc.).,

The coaptation element 520 can include one or more paddle securing recesses 523 and one or more clasp securing recesses 525 extending inwardly into outer portions of the coaptation element 520. The paddle securing recesses 523 can be sized and shaped to at least partially receive or secure the paddle portion 506 to the coaptation element 520. The clasp securing recesses 525 can be disposed distal to or below the paddle securing recesses 523 and can be sized and shaped to at least partially receive or secure the attachment portion or gripping members 510 to the coaptation element 520. However, the connection of the coaptation element 520, the attachment portion or gripping members 510, and the paddle portion 506 can take any configuration. For example, the coaptation element 520 can include a singular paddle securing recess 523 and a single clasp securing recess 525, each extending around the coaptation element 520, or the coaptation element 520 can be integral with the attachment portion or gripping members 510 and/or the paddle portion 506.

As shown in FIGS. 74A through 74D, the coaptation element 520 can include two or more passages 524 on either side of the boss or connection portion 522. The passages 524 can be angled or partially L-shaped with a passage inlet 527 in the proximal or top portion of the coaptation element 520 and one or more passage outlets 529 in the outer or side portion of the spacer or coaptation element 520. The passage inlet 527 and at least part of the passages 524 can be sized and shaped to receive one or more components which can open and close the valve repair device 500. The passage outlet 529 is disposed proximally above the paddle securing recess 523 and can also be disposed above the clasp securing recess 525.

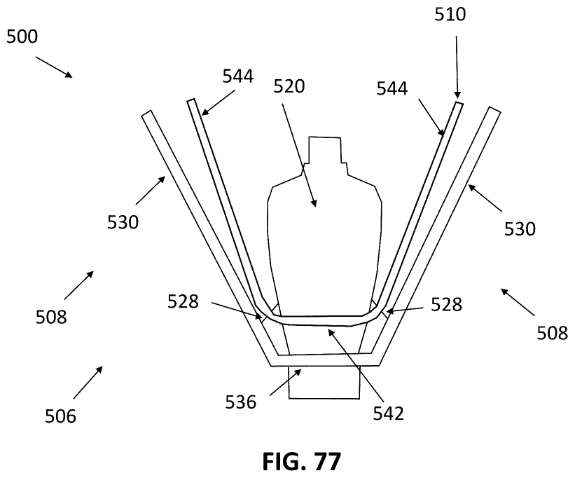

The coaptation element 520 can include an actuator 526 at least partially disposed in the proximal or upper portion of each passage 524. Each actuator 526 can be sized and shaped to securely fit in the respective passage 524. The actuators 526 can be a cap, button, or other component which can be actuated by an actuation element (e.g., actuation wire, shaft, rod, line, etc.) extending from a delivery sheath or system. For example, the actuators 526 can be configured to be engaged or actuated by an actuation element 591 (e.g., actuation shaft, actuation wire, etc.; FIGS. 77-79) to at least partially open the valve repair device 500.

The paddle portion 506 of the valve repair device 500 includes a plurality of paddles 508, each paddle 508 including a paddle 530 and a connection portion 532. The paddles 508 can facilitate the engagement of valve tissue 20, 22 as part of any suitable valve repair system. The paddle 530 can be generally flat with a bulbous or lightbulb-shaped cross-section having a wider distal end and a narrower medial end. The paddle 530 can be a substantially wire frame. However, the paddle 530 can be any suitable shape or configuration. For example, the paddle 530 can be rectangular or any other shape and can be a mesh or solid frame or any other suitable configuration. Each paddle 530 can extend upwardly and radially outwardly from a medial position to a proximal and radially outer end.

The connection portion 532 of the paddle 508 can be used to control the movement of the paddle 530. The connection portion 532 can be moved, controlled, or otherwise maneuvered by an actuation element (e.g., actuation shaft, actuation wire, etc.). The connection portion 532 can be disposed in the narrower, medial portion of the paddle 530 and can be formed by pinching or crimping the frame of the paddle 530. However, the connection portion 532 can be formed in any suitable manner. For example, the connection portion 532 can be a loop, hook, or other connective component which can permit a connection with the paddle 530.

The paddle portion 506 also includes a rounded or elliptical paddle collar 536 which can connect or secure the paddle portion 506 to the coaptation element 520. For example, the paddle collar 536 can be sized and shaped to fit into the paddle securing recesses 523 and around an outer portion of the coaptation element 520. For example, the paddle collar 536 can be sized and shaped to be at least partially snap fit into the paddle securing recesses 523 of the coaptation element 520. The paddle collar 536 and paddle securing recesses 523 can also be sized, shaped, or otherwise configured such that the paddle collar 536 can be at least partially secured in the paddle securing recesses 523 by an interference fit. Each paddle 530 can be connected to the paddle collar 536 such that each paddle 508 (e.g., paddle 530, connection portion 532) can be flexed or pivoted about the paddle collar 536 and articulated, maneuvered, or otherwise articulated independently of the other paddle 508, as described below.

The paddle portion 506 can be configured such that the paddles 530 are biased downwardly and outwardly (e.g., away from the proximal portion of the valve repair device 500). The paddles 530, connection portions 532, and paddle collar 536 can be derived from a single super-elastic sheet, ribbon or wire which can resist plastic deformation. In some implementations, as illustrated, the paddle collar 536 is semi-circular and integral with paddles 530. However, the paddle portion 506 can be configured or connected in any suitable manner. For example, the paddle collar 536 can be circular and separate from the paddles 530 and the paddles 530 can be connected to opposing sides of the paddle collar 536 by a hinge, joint, or other flexible or pivotable connector.

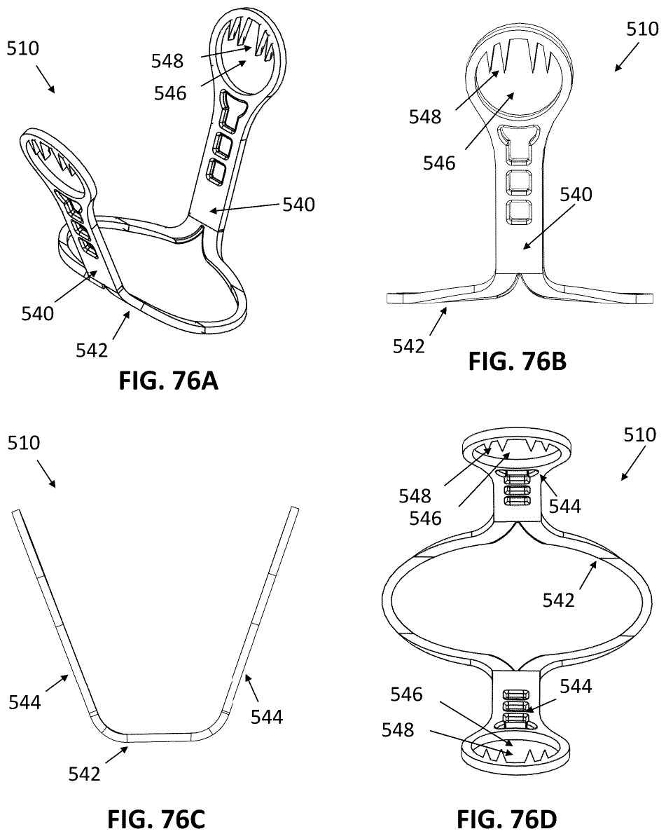

The attachment portion or gripping members 510 can be substantially similar to the attachment portion or gripping members 410 shown in FIGS. 57 through 67. As shown in FIGS. 76A through 76D, the attachment portion or gripping members 510 can include a rounded or elliptical collar 542 which can connect or secure the attachment portion or gripping members 510 to the coaptation element 520. The collar 542 can be sized and shaped to be secured, placed, or otherwise disposed on the coaptation element 520. For example, the collar 542 can be sized and shaped to be at least partially snap fit into the clasp securing recesses 525 of the coaptation element 520. The collar 542 and clasp securing recesses 525 can also be sized, shaped, or otherwise configured such that the collar 542 can be at least partially secured in the clasp securing recesses 525 by an interference fit.

The attachment portion or gripping members 510 can include two or more clasp elements 544 which can facilitate the engagement of valve tissue 20, 22 as part of any suitable valve repair system. Each clasp element 544 can have a narrower portion 540 which is connected to the collar 542 and a wider portion which can include a clasp engagement portion 546 for engagement of the valve tissue 20, 22 as part of any suitable valve repair system. In some implementations, as illustrated, the clasp elements 544 are bulbous with a partially lightbulb or teardrop-shape. However, the clasp elements 544 can have any suitable size, shape, or configuration. For example, the clasp elements 544 can be substantially solid, a wire frame, rectangular, and/or similar to the size, shape, or configuration of the paddles 530.

The clasp elements 544 can extend radially outward and proximally upward from the collar 542. The clasp elements 544 can be integrally formed with the collar 542. The attachment portion or gripping members 510 can be formed from a single, super-elastic sheet, ribbon, or wire such that the attachment portion or gripping members 510 can resist deformation. For example, the attachment portion or gripping members 510 can be formed from a single sheet or piece of material such that the radially outward portions of the clasp elements 544 are urged or biased downwardly or distally. However, the attachment portion or gripping members 510 can have any suitable shape, size, or configuration and the collar 542 and clasp elements 544 can have any suitable connection. For example, the clasp elements 544 can be connected to the collar 542 by a hinge, joint, or other flexible or pivotable connector.

Each clasp element 544 can include one or more optional projections or barbs 548 extending into the clasp engagement portion 546. The barbs 548 can engage the leaflets 20, 22 and secure the valve repair device 500 in the native valve, e.g., native mitral valve, when the device 500 is in the closed position, as described below. The tissue of the leaflet 20, 22 is not pierced by the barbs 548, though in some implementations the barbs 548 can partially or fully pierce through the leaflet 20, 22. In some implementations, as illustrated, the barbs 548 extend radially inward into the clasp engagement portion 546 and in line with the remainder of the clasp element 544. However, the barbs 548 can have any suitable size, shape, orientation, or configuration to secure the valve repair device 500 in the native valve. For example, the barbs 548 can be at an angle or perpendicular to the remainder of the clasp element 544 such that the barbs 548 can engage the tissue of the leaflets 20, 22.

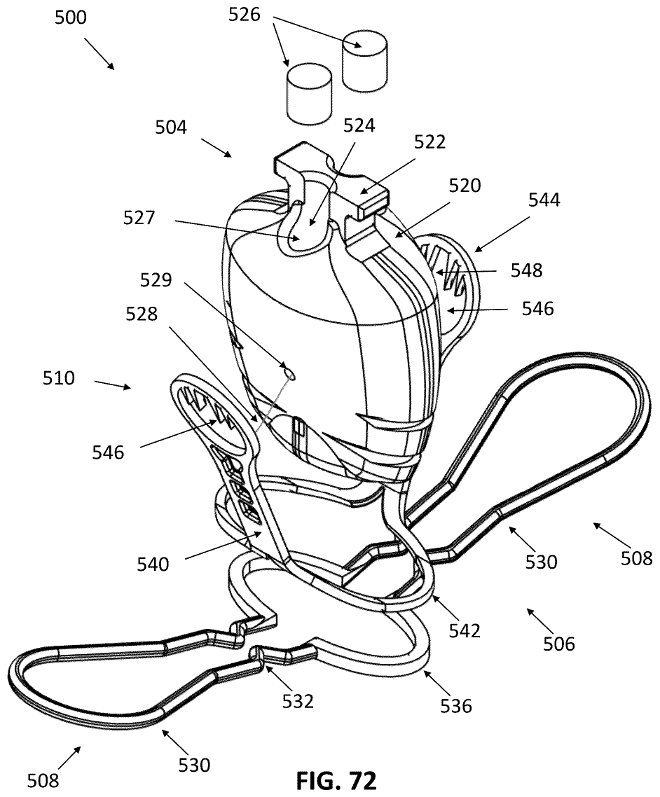

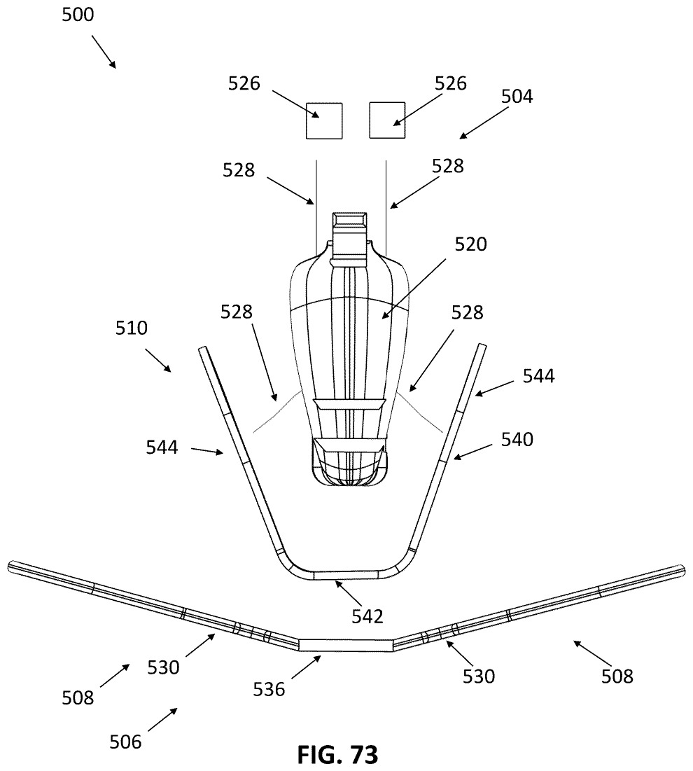

As shown in FIGS. 72 and 73, the coaptation element 520 can also include a connection element (e.g., line, rod, tube, shaft, hypotube, etc.) 528 in each of the passages 524 which can facilitate the opening and closing of the valve repair device 500, as detailed below. Each connection element 528 is disposed between and connected or affixed to one of the actuators 526 and one of the paddles 530 (See FIGS. 77-79). FIGS. 72 and 73 are exploded views, so the connection element 528 is shown as being disconnected and spaced apart from the paddles 530. The connection element 528 can be connected or affixed to the paddle 530 in a wide variety of different ways. For example, the end of the connection element 528 can be looped around or connected or affixed to connection portion 532. In some implementations, as illustrated, the connection elements 528 are sutures or wires. However, the connection elements 528 can be any device or component which can provide a connection between the actuator 526 and the paddle 530. For example, the connection elements 528 can be shape-memory alloys such as Nitinol. In some implementations, as illustrated, the device 500 is depicted as having one connection element 528 in each passage 524. However, the device 500 can have any number of connection elements 528. For example, the device 500 can have two connection elements 528 in each passage 524 with one connection element 528 connected to each side of the paddle 530 and/or the connection portion 532.

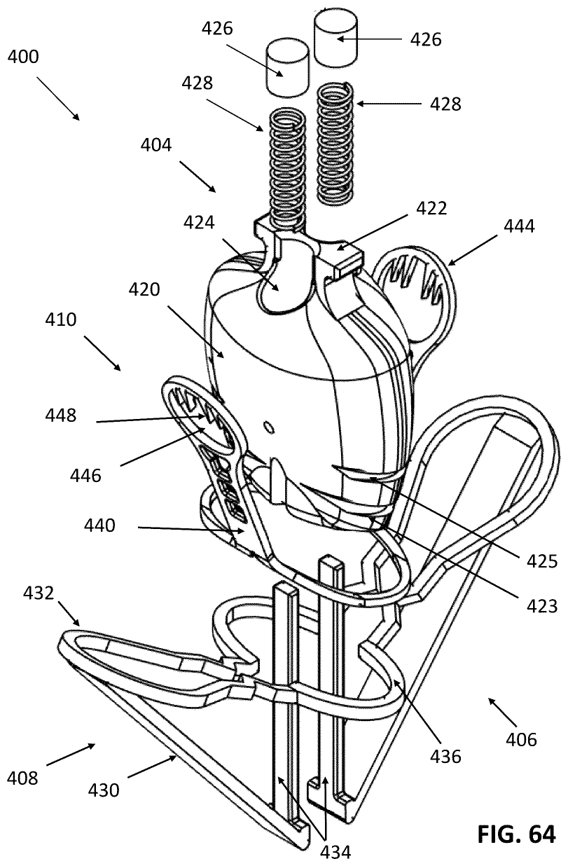

Referring now to FIGS. 77 through 79, the valve repair device 500 can be moved between a closed position and an open position. As shown in FIG. 77, the device 500 can be deployed in the closed position with the paddles 508 (e.g., paddles 530, connection portions 532) pulled proximally upward and radially inward. The connection elements 528 can keep the paddles 530 and connection portions 532 pulled proximally upwardly and inwardly toward the coaptation element 520. For example, the connection elements 528 can provide a tensioning force which opposes the downward or distal biasing force of the paddles 508 and thereby retains the paddles 508 in the closed or retracted position. For example, a spring or other biasing element (see biasing elements 428 in FIG. 64) can bias the actuators 526 to the proximal end of the spacer. As a result, the actuators 526 pull the connection elements 528 to bias the paddles to the closed or retracted position. The clasp elements 544 can be in engagement with the paddles 530.

As shown in FIG. 78, one of the paddles 508 can be moved to the open position with the actuation element (e.g., rod, shaft, tube, etc.) 591. The actuation element 591 can engage one of the actuators 526 and move the actuator 526 downwardly. Moving the actuator 526 distally or downwardly in the passage 524 provides slack to or decreases the tension in the connection element 528. The increased slack in the connection element 528 permits the biasing force of the paddle portion 506 to pivot or flex the paddle 530 distally or downwardly. The downward or distal biasing force of the paddle portion 506 causes the outer portion of the paddle 530 to move distally and radially outwardly creating a tissue receiving gap 452 between the paddle 530 and the clasp element 544.

As shown in FIG. 79, the other paddle 508 can be moved to the open position with one of the actuation elements 591. The same process can be repeated to move the other paddle 530 to create another tissue receiving gap 552 between the other paddle 530 and other clasp element 544.

While the valve repair device 500 is in the partially open position (e.g., one paddle is open in FIG. 78) or the fully open position (e.g., both paddles are open in FIG. 79), the valve repair device 500 can be maneuvered, positioned, or otherwise moved to the desired position. The valve repair device 500 can be maneuvered or otherwise moved such that the native leaflets 20, 22 are in one of the tissue receiving gaps 552 on either or both sides of the valve repair device 500. For example, the position or movement of the valve repair device 500 can be controlled by a connection or engagement with the boss or connection portion 522 of the coaptation element 520. For example, the boss or connection portion 522 can be sized, shaped, or otherwise configured to be engaged or positioned by an actuation element (e.g., actuation shaft, actuation rod, actuation tube, actuation wire, actuation line, etc.) 591, and/or to be removably attached to a delivery system, collar, or capture mechanism (e.g. one or more of a clamp, clip, pin, suture, line, lasso, noose, snare, buckle, lock, latch, etc.).,

Once one of the paddles 508 is in place in the native valve, e.g., native mitral valve, with the native leaflets 20, 22 in the tissue receiving gaps 552, the respective paddle 508 can be closed. For example, the valve repair device 500 can be closed to capture the native leaflets 20, 22, such as between the paddle 530 and the clasp element 544. The actuation element 591 can be retracted proximally or upwardly to disengage the actuator 526. Disengaging the actuator 526 causes the actuator 526 and connection element 528 to retract proximally or upwardly into the passage 524 and toward the proximal portion 501 of the valve repair device 500. For example, the coaptation element 520 can include a spring or biasing element, such as the biasing element 428 described in FIGS. 57 through 67, which biases the actuator 526 toward the proximal portion 501 of the valve repair device 500. The upward movement of the actuator 526 and connection element 528 increases the tensioning force applied to the connection portion 532 and pulls the paddle 530 proximally and radially inward toward the clasp element 544. The paddle 530 can move upwardly and inwardly to at least partially engage the clasp element 544. The native leaflets 20, 22 can be secured by the distal or downward bias of the clasp element 544 and the upward tensioning force applied to the paddle 530 by the connection element 528.

Once the native leaflets 20, 22 are secured on one side of the valve repair device 500, the valve repair device 500 can be positioned or repositioned such that the native leaflets 20, 22 are disposed in the tissue receiving gap 552 on the other side of the valve repair device 500. The closing process can be repeated for the other paddle 508 when the native leaflets 20, 22 are in place on the other side of the valve repair device 500, such as in the tissue receiving gap 552.

While the process has been described as opening both paddles 508 and closing each paddle 508 in turn, the device 500 can be opened, positioned, and closed in other manners. For example, one paddle 508 can be opened, positioned, and closed in place and then the other paddle 508 can be opened, positioned, and closed in place or both paddles 508 can be opened and positioned and then simultaneously closed in place.

Once the valve repair device 500 is closed in the desired position, the valve repair device 500 can be released from the delivery system or capture mechanism, and the delivery system, capture mechanism, and actuation element 591 can be withdrawn and removed. The native leaflets 20, 22 can be secured or engaged by the clasp engagement portion 546, such as by the barbs 548 of the clasp elements 544.

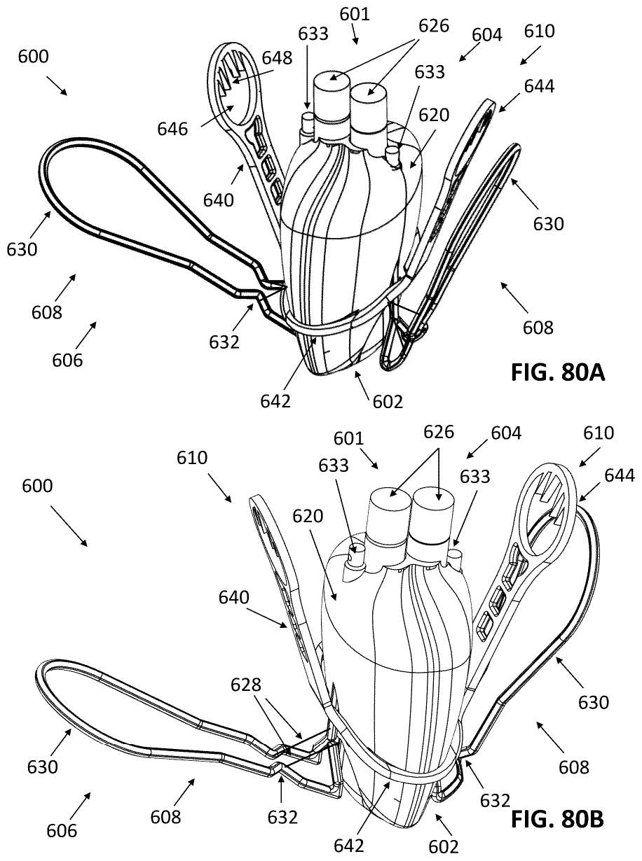

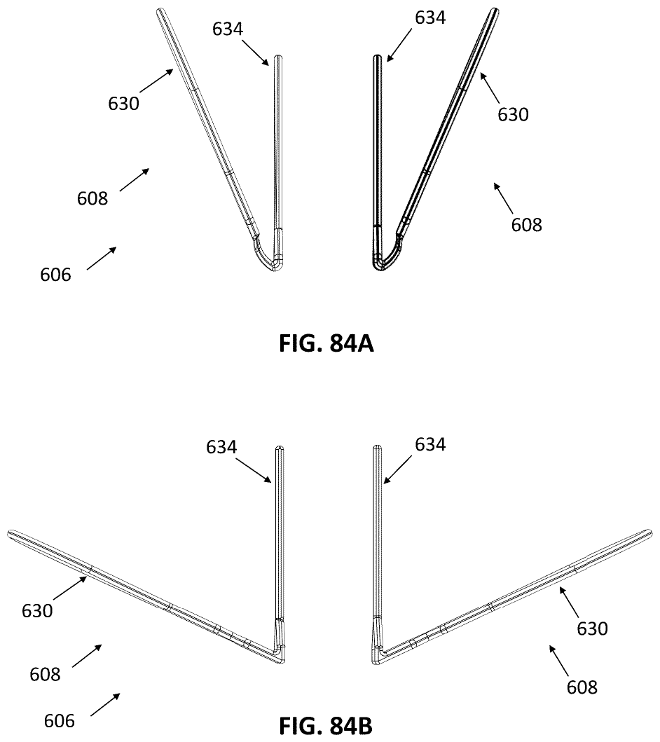

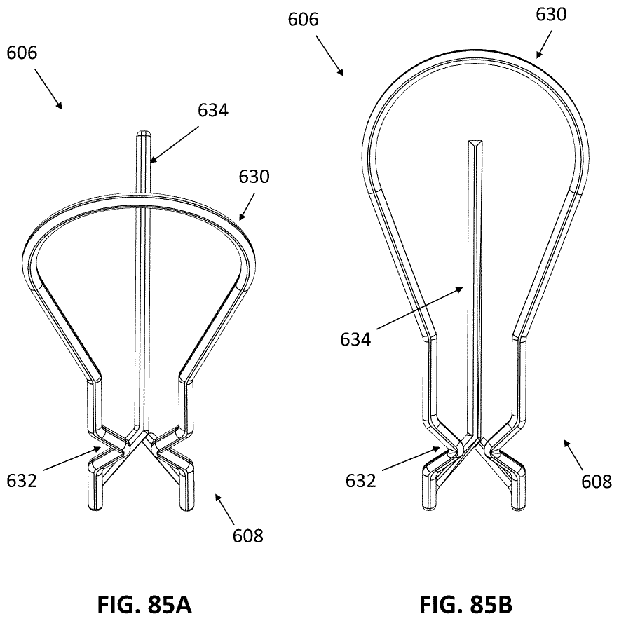

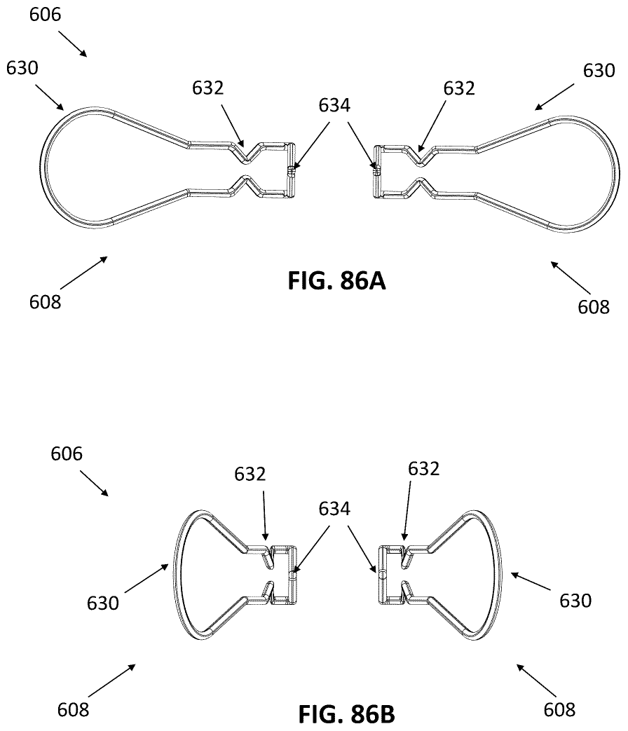

Referring now to FIGS. 80A through 94, an example of a device (e.g., an implantable prosthetic device, a prosthetic spacer device, a valve repair device, etc.) 600 is shown. The valve repair device 600 is one of the many different configurations that the device 100 that is schematically illustrated in FIGS. 8-15 can take. The device 600 can include any other features for an implantable prosthetic device discussed in the present application or any of the applications that are incorporated herein by reference, and the device 600 can be positioned to engage valve tissue 20, 22 as part of any suitable valve repair system (e.g., any valve repair system disclosed in the present application or any of the applications that are incorporated herein by reference). The various components of the valve repair device 600 can be made at any suitable size to accommodate different size patient anatomy.

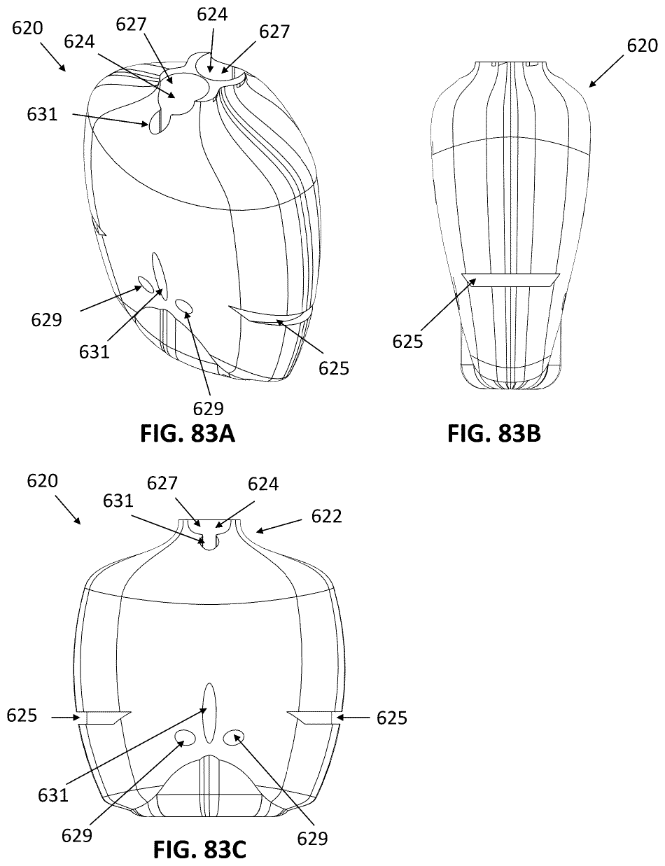

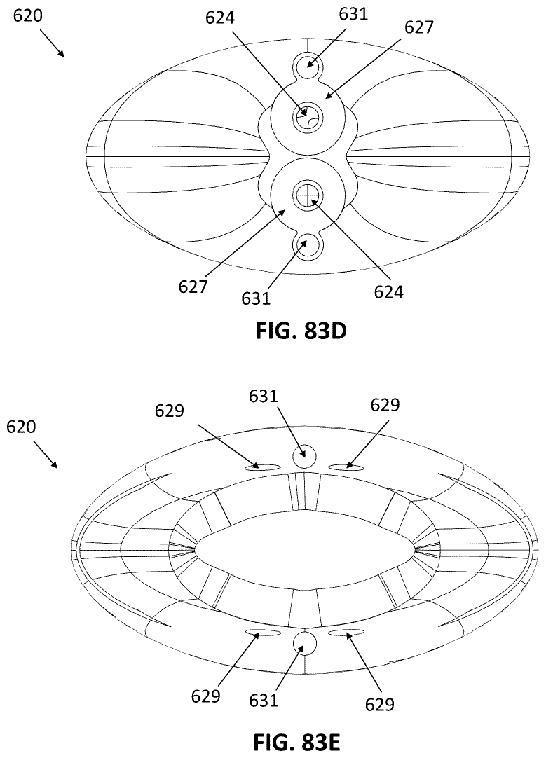

The valve repair device 600 extends from a proximal portion 601 to a distal portion 602 and can include an optional coaptation portion 604 and an anchor portion. In some implementations, the anchor portion comprises one or more paddle portions 606 and/or an attachment portion or gripping members 610. The coaptation portion 604 includes a coaptation element 620 (e.g., spacer, coaption element, gap filler, membrane, sheet, plug, wedge, and balloon etc.) for implantation between the leaflets 20, 22 of the native valve. The coaptation element 620 has a generally elongated and round shape. In particular, the coaptation element 620 has an elliptical shape or cross-section when viewed from above (FIG. 83D) and has a tapered shape or cross-section when seen from a front view (e.g., FIG. 83C). A blend of these three geometries can result in the three-dimensional shape of the illustrated coaptation element 620 that achieves the benefits described herein. The round shape of the coaptation element 620 can also be seen, when viewed from above, to substantially follow or be close to the shape of the collars for the attachment portion or gripping members and the paddle portion, as described below.

As shown in FIGS. 83A and 83B, the coaptation element 620 can include a boss or connection portion 622 extending upwardly from a proximal portion of the coaptation element 620. The boss or connection portion 622 can be sized and shaped to be secured and/or manipulated by a user during an implantation operation. The boss or connection portion 622 can be sized and shaped such that the coaptation element 620 can be retained, deployed, positioned, recaptured, repositioned, and/or redeployed during the implantation operation. For example, the boss or connection portion 622 can be sized, shaped, or otherwise configured to be engaged or positioned by an actuation element (e.g., actuation shaft, actuation rod, actuation tube, actuation wire, actuation line, etc.) 691, and/or to be removably attach to a delivery system or capture mechanism. The boss or connection portion 622 can at least partially comprise one or more actuators, as detailed below.

The coaptation element 620 can include one or more clasp securing recesses 625 extending inwardly into outer portions of the coaptation element 620. The clasp securing recesses 625 can be sized and shaped to at least partially receive or secure the attachment portion or gripping members 610 to the coaptation element 620. However, the connection of the coaptation element 620 and the attachment portion or gripping members 610 can take any configuration. For example, the coaptation element 620 can include a single clasp securing recess 625 extending around the coaptation element 620 or the coaptation element 620 can be integral with the attachment portion or gripping members 610.

As shown in FIGS. 83A through 83E, the coaptation element 620 can include two or more passages 624 on either side of the boss or connection portion 622. The passages 624 can be angled or partially L-shaped with a passage inlet 627 in the proximal or top portion of the coaptation element 620 and two passage outlets 629 in the outer or side portion of the coaptation element 620. The passage 624 can have a substantially vertical portion extending downwardly from the passage inlet 627 which branches into two radially extending or angled passages which lead to the two passage outlets 629. The passage inlet 627 and at least part of the passages 624 can be sized and shaped to receive one or more components which can open and close one of the paddle portions 606. The passage outlets 629 can be disposed below the clasp securing recess 625.

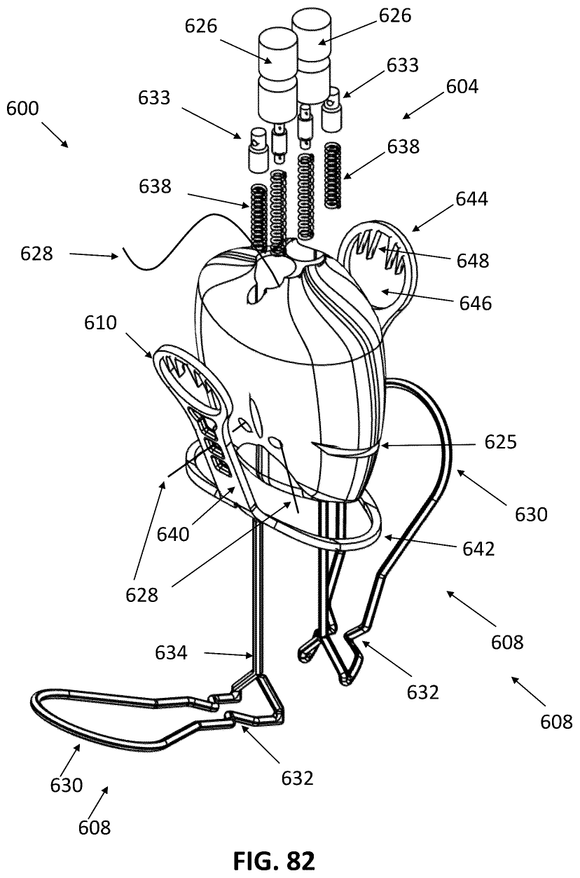

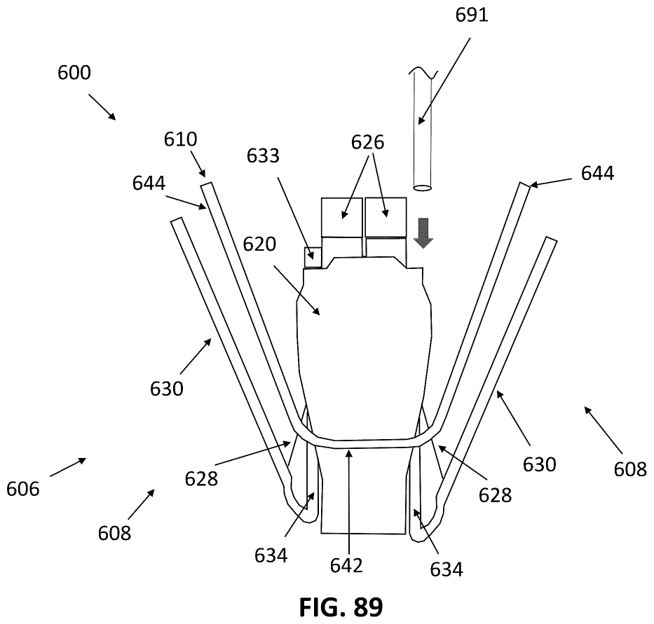

Referring to FIG. 82, the coaptation element 620 can include a first actuator 626 disposed above or partially extending into the proximal or upper portion of each passage 624. Each first actuator 626 can be sized and shaped to fit at least partially into the respective passage 624. The first actuators 626 can be a cap, button, or other component which can be actuated by an actuation element (e.g., actuation wire, shaft, rod, line, etc.) extending from a delivery sheath or system, such as delivery system 202 (See FIGS. 38-49). For example, the first actuators 626 can be configured to be engaged or actuated by an actuation element 691 (e.g., actuation shaft, actuation wire, etc.; FIGS. 89-94) to at least partially open the valve repair device 600.

The coaptation element 620 can also include two or more passages 631 that are each disposed radially outwardly from the passage inlets 627. Each passage 631 can extend longitudinally through the coaptation element 620 from the proximal portion to the distal portion. The passages 631 can be narrower than the passage inlet 627. The passages 631 can be sized and shaped to receive one or more components which can raise and lower one of the paddle portions 606, as detailed below. The passage outlets 629 can be disposed symmetrically on either side of the passage 631.

The coaptation element 620 can include a second actuator 633 disposed above or partially extending into the proximal or upper portion of each passage 631 when the paddle portion 606 is in the raised position, as detailed below. Each second actuator 633 can be sized and shaped to fit in the respective passage 631. The second actuators 633 can be a cap, button, or other component which can be actuated by an actuation element (e.g., actuation wire, shaft, rod, line, etc.) extending from a delivery sheath or system. For example, the second actuators 633 can be configured to be engaged or actuated by one of the actuation elements 691 (e.g., actuation wire, actuation shaft, rod, line, etc.) to at least partially open the valve repair device 600.

The paddle portions 606 of the valve repair device 600 each include a paddle 608 with a paddle 630 and a paddle extension shaft 634. The paddle extension shaft 634 can be a substantially vertical shaft which can be at least partially received in one of the passages 631 of the coaptation element 620. Each paddle extension shaft 634 can be partially secured in one of the passages 631 of the coaptation element 620. The paddle extension shafts 634 can be disposed in the passages 631 such that the remainder of the valve repair device 600 can twist, pivot, or otherwise rotate about the paddle extension shaft 634, such as when the paddle 608 is in engagement with valve tissue 20, 22, as described below. For example, the coaptation element 620, the other paddle portion 606, and/or the attachment portion or gripping members 610 can rotate about a longitudinal axis extending through one of the paddle extension shafts 634 when the paddle 608 associated with the paddle extension shaft 634 is in engagement with the valve tissue 20, 22. Such control over the valve repair device 600 can permit a user to then position the valve repair device 600 such that the other paddle portion 606 can engage the valve tissue 20, 22, as detailed below.

The paddles 608 can facilitate the engagement of valve tissue 20, 22 as part of any suitable valve repair system. The paddle 630 can be generally flat with a bulbous or lightbulb-shaped cross-section having a wider distal end and a narrower medial end. The paddle 630 can be a substantially looped wire frame. However, the paddle 630 can be any suitable shape or configuration. For example, the paddle 630 can be rectangular or any other shape and can be a mesh or solid frame or any other suitable configuration. Each paddle 630 can extend upwardly and outwardly from the distal end of one of the paddle extension shafts 634. The paddle 630 can be flexibly connected to the paddle extension shaft 634 such that the paddle 630 can be at least partially pivotable about the end of the paddle extension shaft 634. For example, the paddle 630 can be connected to the paddle extension shaft 634 by a hinge, joint, or other pivotable connector or can be flexibly integral with the distal end of the paddle extension shaft 634.

The paddles 608 can optionally include a connection portion 632 which can be used to control the movement of the paddle 630. The connection portion 632 can be moved, controlled, or otherwise maneuvered by an actuation element (e.g., actuation shaft, actuation wire, etc.). The connection portion 632 can be disposed in the narrower, medial portion of the paddle 630 and can be formed by pinching or crimping the frame of the paddle 630. However, the connection portion 632 can be formed in any suitable manner. For example, the connection portion 632 can be a loop, hook, or other connective component which can permit a connection with the paddle 630.

The paddle portion 606 can be configured such that the paddles 630 are biased downwardly and outwardly (e.g., away from the proximal portion of the valve repair device 600). The paddles 630, connection portions 632, and paddle extension shaft 634 can be derived from a single super-elastic sheet, ribbon or wire which can resist plastic deformation. However, the paddle portion 606 can be configured or connected in any suitable manner. For example, the paddle extension shaft 634, paddle 630, and connection portion 632 can be separate components which are connected by pivotable or flexible connectors.

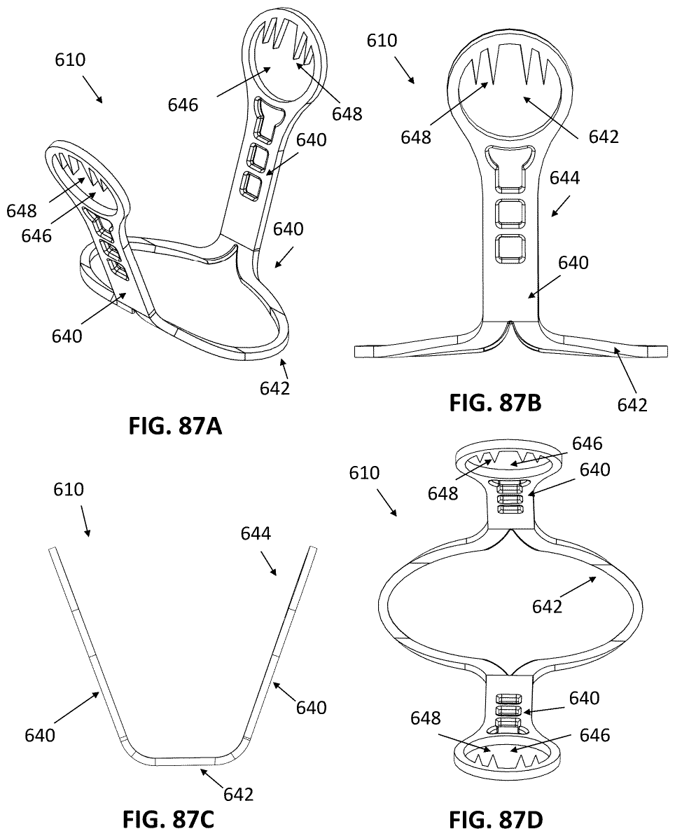

The attachment portion or gripping members 610 can be substantially similar to the attachment portion or gripping members 410 shown in in FIGS. 57 through 67 or the attachment portion or gripping members 510 shown in FIGS. 68 through 79. As shown in FIGS. 87A through 87D, the attachment portion or gripping members 610 can include a rounded or elliptical collar 642 which can connect or secure the attachment portion or gripping members 610 to the coaptation element 620. The collar 642 can be sized and shaped to be secured, placed, or otherwise disposed on the coaptation element 620. For example, the collar 642 can be sized and shaped to be at least partially snap fit into the clasp securing recesses 625 of the coaptation element 620. The collar 642 and clasp securing recesses 625 can also be sized, shaped, or otherwise configured such that the collar 642 can be at least partially secured in the clasp securing recesses 625 by an interference fit.

The attachment portion or gripping members 610 can include two or more clasp elements 644 which can facilitate the engagement of to engage valve tissue 20, 22 as part of any suitable valve repair system. Each clasp element 644 can have a narrower radially proximal portion 640 which is connected to the collar 642 and a wider radially distal portion which can include a clasp engagement portion 646 for engagement of the valve tissue 20, 22 as part of any suitable valve repair system. In some implementations, as illustrated, the clasp elements 644 are substantially flat with a bulbous or lightbulb- or teardrop-shaped cross-section. However, the clasp elements 644 can have any suitable size, shape, or configuration. For example, the clasp elements 644 can be substantially solid, a wire frame, rectangular, and/or similar to the size, shape, or configuration of the paddles 630.

The clasp elements 644 can extend radially outward and proximally upward from the collar 642. The clasp elements 644 can be integrally formed with the collar 642. The attachment portion or gripping members 610 can be formed from a single, super-elastic sheet, ribbon, or wire such that the attachment portion or gripping members 610 can resist deformation. For example, the attachment portion or gripping members 610 can be formed from a single sheet or piece of material such that the radially outward portions of the clasp elements 644 are urged or biased downwardly or distally. However, the attachment portion or gripping members 610 can have any suitable shape, size, or configuration and the collar 642 and clasp elements 644 can have any suitable connection. For example, the clasp elements 644 can be connected to the collar 642 by a hinge, joint, or other flexible or pivotable connector.

Each clasp element 644 can include one or more optional projections or barbs 648 extending into the clasp engagement portion 646. The optional barbs 648 can engage the leaflets 20, 22 and secure the valve repair device 600 in the native valve, e.g., native mitral valve, when the device 600 is in the closed positions, as described below. The tissue of the leaflet 20, 22 is not pierced by the barbs 648, though in some implementations the barbs 648 can partially or fully pierce through the leaflet 20, 22. In some implementations, as illustrated, the barbs 648 extend radially inward into the clasp engagement portion 646 and in line with the remainder of the clasp element 644. However, the barbs 648 can have any suitable size, shape, orientation, or configuration to secure the valve repair device 600 in the native valve. For example, the barbs 648 can be at an angle or perpendicular to the remainder of the clasp element 644 such that the barbs 648 can engage the tissue of the leaflets 20, 22.

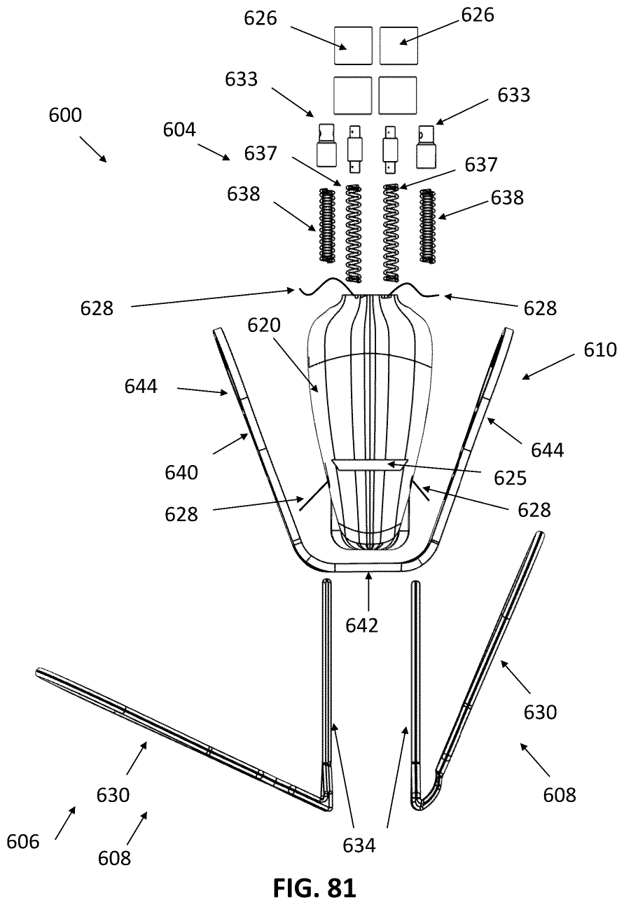

As shown in FIGS. 81 through 82, the coaptation element 620 can also include one or more connection elements 628 in each of the passages 624 which can facilitate the opening and closing of the valve repair device 600, as detailed below. Each connection element (e.g., line, shaft, tube, hypotube, wire, etc.) 628 can be disposed between and connected or affixed to one of the first actuators 626 and the corresponding paddle 630. When the device 600 is in the closed position, the connection element 628 can provide a tensioning force to the paddle 630 which keeps the paddle 630 in the upright or closed position, and when the first actuator 626 is engaged or depressed into the passage 624, the connection element 628 can provide slack or decrease the tension which allows the paddle 630 to open or flex radially outward, as described below. The connection element 628 can be connected or affixed to the paddle 630. For example, the end of the connection element 628 can be looped around or connected or affixed to connection portion 632. In some implementations, as illustrated, the connection elements 628 are sutures or wires. However, the connection elements 628 can be any device or component which can provide a connection between the actuator 626 and the paddle 630. For example, the connection elements 628 can be shape-memory alloys such as Nitinol. In some implementations, as illustrated, the connection element 628 has a singular portion at the passage inlet 627 and two ends, each extending out from one of the passage outlets 629. However, any number and configuration of connection elements 628 can be used. For example, each passage 624 can include two connection elements 628, each extending through one of the passage outlets 629.

As shown in FIGS. 81 and 82, the coaptation element 620 can also include a first biasing element (e.g., spring, band, compressible material, compressible fluid, etc.) 637 in each of the passages 624 which can oppose the forces output from the paddle portion 606. For example, the first biasing element 637 can exert a biasing force which keeps the first actuator 626 in the upright or undepressed state when the device 600 is in the closed position. Each first biasing element 637 can be disposed between and connected or affixed to the first actuator 626 and a proximal portion of the one or more connection elements 628. In some implementations, as illustrated, the first biasing elements 637 are coil springs. However, the first biasing elements 637 can be any device or component which can provide a biasing force. For example, the first biasing elements 637 can be leaf springs, shape-memory alloys such as Nitinol, or any other biasing device.

The coaptation element 620 can also include a second biasing element (e.g., spring, band, compressible material, compressible fluid, etc.) 638 in each of the passages 631 which can oppose the forces output from the paddle portion 606. The second biasing element 638 can keep the paddle extension shaft 634 pulled proximally into the passage 631 of the coaptation element 620. For example, the second biasing element 638 can exert a biasing force which keeps the second actuator 633 in the undepressed state and the paddle extension shaft 634 in the proximal or retracted position when the device 600 is in the closed or retracted position. Each biasing element 638 can be disposed between and connected or affixed to the second actuators 633 and a proximal portion of one of the paddle extension shafts 634. In some implementations, as illustrated, the second biasing elements 638 are coil springs. However, the second biasing elements 638 can be any device or component which can provide a biasing force. For example, the second biasing elements 638 can be leaf springs, shape-memory alloys such as Nitinol, or any other biasing device.

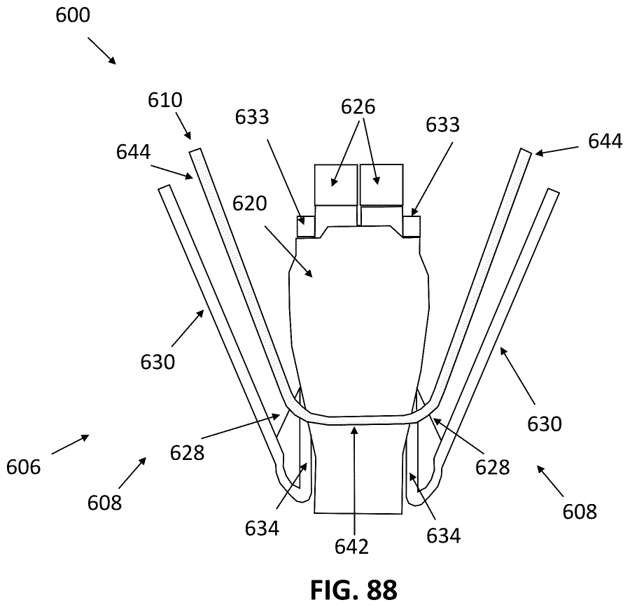

Referring now to FIGS. 88 through 94, the valve repair device 600 can be moved between a closed position and an open position. As shown in FIG. 88, the device 600 can be deployed in the closed position with the paddles 608 (e.g., paddles 630, connection portions 632, and paddle extension shaft 634) pulled proximally upward and radially inward. The connection elements (e.g., lines, rods, tubes, hypotubes, sutures, etc.) 628 can keep the paddles 630 and connection portions 632 pulled proximally upwardly and inwardly toward the coaptation element 620. For example, the connection elements 628 can provide a tensioning force which opposes the downward or distal biasing force of the paddles 608 and thereby retains the paddles 630 of the paddles 608 in the closed or retracted position. The second biasing element 638 can provide a biasing force which keeps the paddle extension shafts 634 in the upright or retracted position. The clasp elements 644 can be in engagement with the paddles 630.

As shown in FIG. 89, one of the paddles 608 can be moved to the protracted position with one of the actuation elements 691. The actuation element 691 can engage one of the second actuators 633 and move the second actuator 633 downwardly. Moving the second actuator 633 distally or downwardly in the passage 631 counteracts the biasing force of the second biasing element 638 and exerts a downward force on the paddle extension shaft 634 which moves the paddle extension shaft 634 at least partially distally or downwardly out of the passage 631.

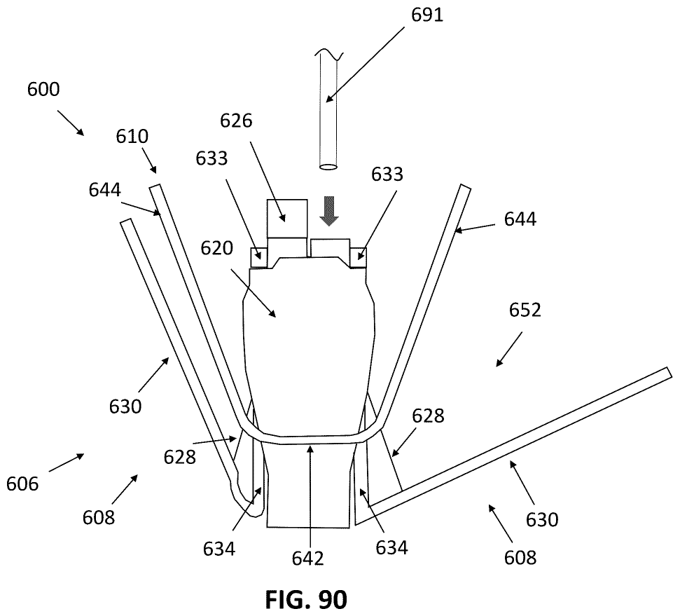

As shown in FIG. 90, one of the paddles 608 can be moved to the open position with one or more actuation elements 691. The actuation element 691 can engage one of the first actuators 626 and move the first actuator 626 downwardly or distally. Moving the first actuator 626 downwardly in the passage 624 provides slack to or decreases the tension in the connection element 628. The increased slack in the connection element 628 permits the biasing force of the paddle portion 606 to pivot or flex the paddle 630 distally or downwardly. The downward or distal biasing force of the paddle portion 606 causes the outer portion of the paddle 630 to move distally and radially outwardly creating a tissue receiving gap 652 between the paddle 630 and the clasp element 644.

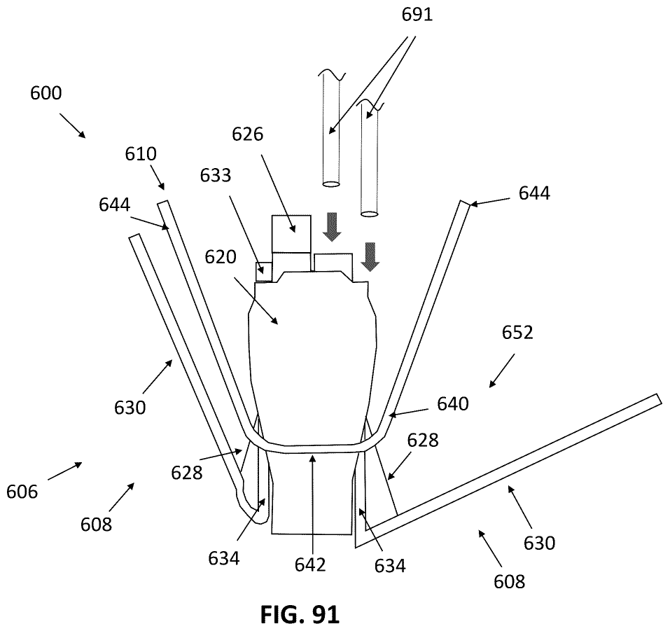

As shown in FIG. 91, the device 600 can be moved to the partially open position with one of the paddles 608 moved to the protracted and open position with one or more actuation elements 691. The one or more actuation elements 691 can engage one of the second actuators 633 (as shown in FIG. 89) and the first actuator 626 on the same side of the coaptation element 620 as the engaged second actuator 633 (as shown in FIG. 90). Moving the first and second actuators 626, 633 downwardly and distally causes the paddle extension shaft 634 to move distally or downwardly out of the passage 631 and causes the outer portion of the paddle 630 to move distally and radially outwardly creating a tissue receiving gap 652 between the paddle 630 and the clasp element 644, as described above. Either the first actuator 626 or the second actuator 633 can be engaged and actuated first or the first and second actuators 626, 633 can be engaged and actuated simultaneously.

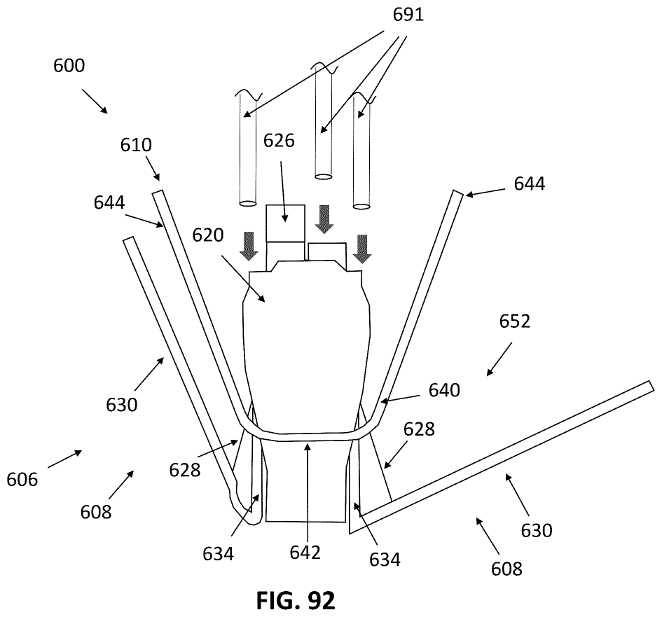

The same process can be repeated to move the other paddle 630 to create another tissue receiving gap 652 between the other paddle 630 and other clasp element 644. As shown in FIG. 92, the other paddle 608 can be moved to the protracted position with one of the actuation elements 691. The actuation element 691 can engage the second actuator 633 and move the second actuator 633 downward. Moving the second actuator 633 distally or downwardly in the passage 631 counteracts the biasing force of the second biasing element 638 and exerts a downward force on the paddle extension shaft 634 which moves the paddle extension shaft 634 at least partially distally or downwardly out of the passage 631.

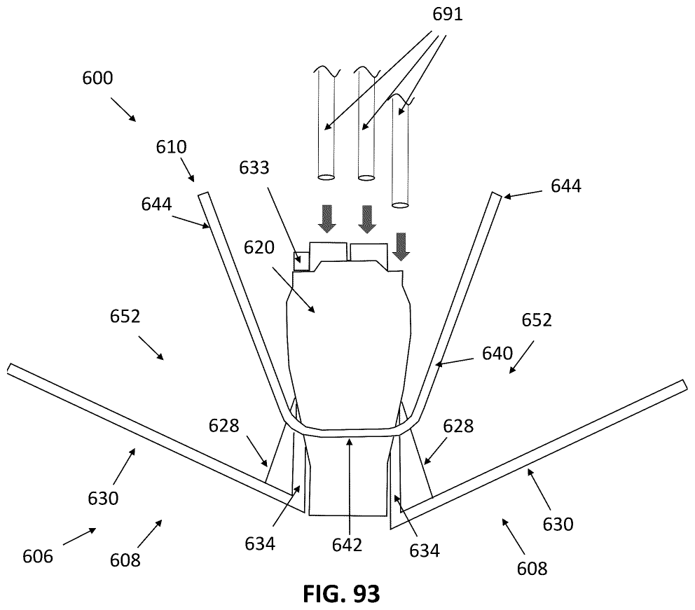

As shown in FIG. 93, the other paddle 608 can be moved to the open position with one or more actuation elements 691. The actuation element 691 can engage the other first actuator 626 and move the first actuator 626 downwardly or distally. Moving the first actuator 626 downwardly in the passage 624 provides slack to or decreases the tension in the connection element 628. The increased slack in the connection element 628 permits the biasing force of the paddle portion 606 to pivot or flex the paddle 630 distally or downwardly. The downward or distal biasing force of the paddle portion 606 causes the outer portion of the paddle 630 to move distally and radially outwardly creating a tissue receiving gap 652 between the paddle 630 and the clasp element 644.

As shown in FIG. 89, one of the paddles 608 can be moved to the protracted position with one of the actuation elements 691. The actuation element 691 can engage one of the second actuators 633 and move the second actuator 633 downward. Moving the second actuator 633 distally or downwardly in the passage 631 counteracts the biasing force of the second biasing element 638 and exerts a downward force on the paddle extension shaft 634 which moves the paddle extension shaft 634 distally or downwardly out of the passage.

One or both of the paddles 608 can be moved to the open position with one or more actuation elements 691. The actuation element 691 can engage one of the first actuators 626 and move the first actuator 626 downwardly or distally. Moving the first actuator 626 downwardly in the passage 624 provides slack to or decreases the tension in the connection element 628. The increased slack in the connection element 628 permits the biasing force of the paddle portion 606 to pivot or flex the paddle 630 distally or downwardly. The downward or distal biasing force of the paddle portion 606 causes the outer portion of the paddle 630 to move distally and radially outwardly creating a tissue receiving gap 652 between the paddle 630 and the clasp element 644.

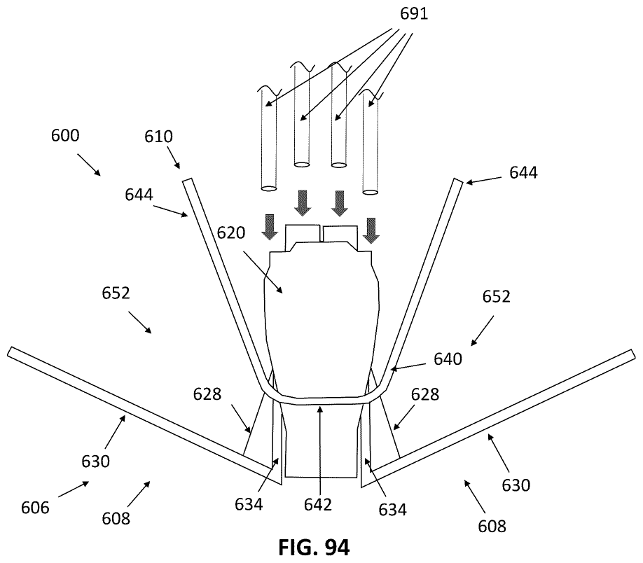

As shown in FIG. 94, the device 600 can be moved to the fully open position with both paddles 608 moved to the protracted and open position with one or more actuation elements 691. The one or more actuation elements 691 can engage the other second actuator 633 (as shown in FIG. 92) and the other first actuator 626 on the same side of the coaptation element 620 (as shown in FIG. 93). Moving the first and second actuators 626, 633 downwardly and distally causes the paddle extension shaft 634 to move distally or downwardly out of the passage 631 and causes the outer portion of the paddle 630 to move distally and radially outwardly creating a tissue receiving gap 652 between the paddle and the clasp element 644, as described above. Either the first actuator 626 or the second actuator 633 can be engaged and actuated first or the first and second actuators 626, 633 can be actuated simultaneously.

While the valve repair device 600 is in a partially open position (e.g., FIG. 91) or a fully open position (e.g., FIG. 94), the valve repair device 600 can be maneuvered, positioned, or otherwise moved to the desired position. The valve repair device 600 can be maneuvered or otherwise moved such that the native leaflets 20, 22 are in one of the tissue receiving gaps 652 on either or both sides of the valve repair device 600. For example, the position or movement of the valve repair device 600 can be controlled by a connection or engagement with the boss or connection portion 622 of the coaptation element 620. For example, the boss or connection portion 622 can be sized, shaped, or otherwise configured to be engaged or positioned by an actuation element, such as actuation element 691, and/or to be removably attached to a delivery system, collar, or capture mechanism, such as delivery system 202 (See FIGS. 38-49).

Once one of the paddles 608 is in place in the native valve, e.g., native mitral valve, with the native leaflets 20, 22 in the tissue receiving gap 652, the respective paddle 608 can be closed. For example, the valve repair device 600 can be closed to capture the native leaflets 20, 22, such as between the paddle 630 and the clasp element 644. The actuation element 691 can be retracted proximally or upwardly to disengage the first and second actuators 626, 633. Disengaging the first and second actuators 626, 633 causes the actuators 626, 633, connection element 628, and paddle extension shaft 634 to retract proximally or upwardly toward the proximal portion 601 of the valve repair device 600 and paddle 630 to flex or retract radially inwardly toward the coaptation element 620. For example, releasing the first and second actuators 626, 633 causes the first and second biasing elements 637, 638 to push the first and second actuators 626, 633 toward the proximal portion 601 of the valve repair device 600 and the clasp element 644. The upward movement of the first actuator 626 increases the tension of the connection element 628 which pulls the connection portion 632 and paddle 630 toward the coaptation element 620. The upward movement of the second actuator 633 pulls the paddle extension shaft 634 proximally or upwardly back into the passage 631 of the coaptation element 620. The native leaflets 20, 22 can be secured by the distal or downward bias of the clasp element 644 and the upward tensioning force applied to the paddle 630 by the connection element 628 and the first and second biasing elements 637, 638. The first and second biasing elements 637, 638 can keep the device 600 in the closed position (FIG. 88) with the native leaflets 20, 22 secured in the tissue receiving gap 652.

Once the native leaflets 20, 22 are secured on one side of the valve repair device 600, the valve repair device 600 can be repositioned such that the native leaflets 20, 22 are disposed in the tissue receiving gap 652 on the other side of the valve repair device 600. The closing process can be repeated for the other paddle 608 when the native leaflets 20, 22 are in place on the other side of the valve repair device 600, such as in the tissue receiving gap 652. While the process has been described as opening both paddle portions 606 and closing each paddle portion 606 in turn, the device 600 can be opened, positioned, and closed in other manners. For example, one paddle portion 606 can be opened, positioned, and closed in place and then the other paddle portion 606 can be opened, positioned, and closed in place or both paddle portions 606 can be opened and positioned and then simultaneously closed in place.

Once the valve repair device 600 is closed in the desired position, the valve repair device 600 can be released from the delivery system or capture mechanism, such as delivery system 202 (See FIGS. 38-49), and the delivery system, capture mechanism, and the actuation elements 691 can be withdrawn and removed. The native leaflets 20, 22 can be secured or engaged by the clasp engagement portion 646, such as by the barbs 648 of the clasp elements 644.

In some implementations, the valve repair device or implant can be configured such that the paddles can transition from a substantially freely rotatable configuration, such as during delivery and deployment, to a substantially secured configuration, such as after the device has been secured to the leaflets of the native valve. For example, the device can include a pass over-center mechanism which allows rotation of the paddles during delivery and deployment of the device and which maintains the paddles in a closed position or configuration after the device has been deployed or implanted in the native heart.

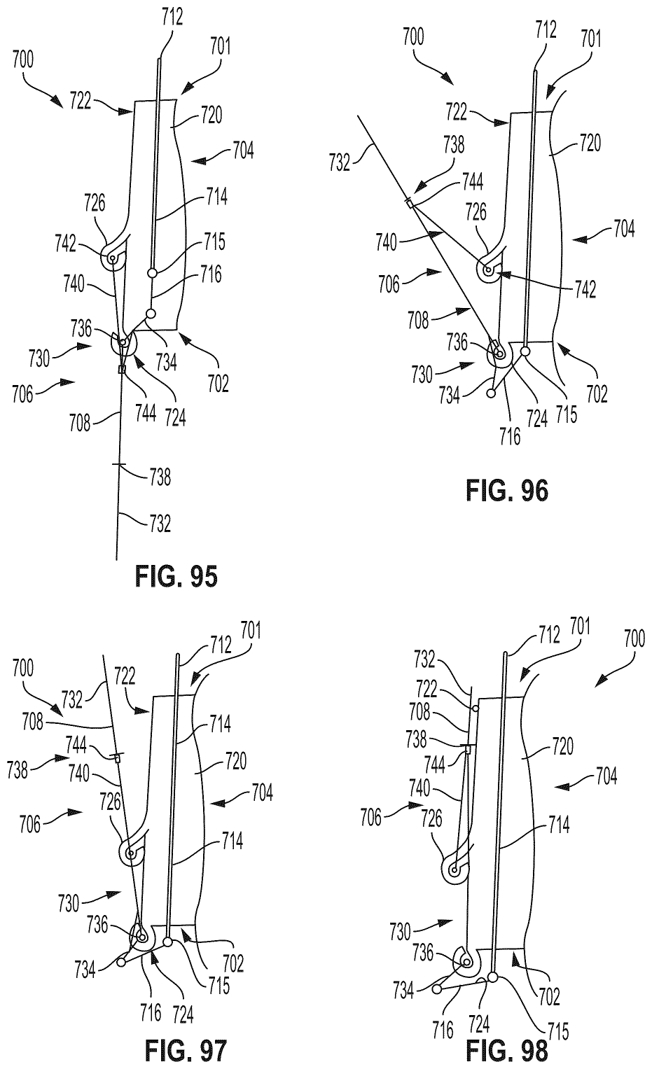

Referring now to FIGS. 95 through 98, an example of a device (e.g., an implantable prosthetic device, a prosthetic spacer device, a valve repair device, etc.) 700 is schematically illustrated. In some implementations, the device 700 can include a pass over-center mechanism which allows rotation anchor portions (e.g., of paddles of an anchor portion and/or of gripping members of an anchor portion) during delivery and deployment of the device and which maintains the anchor portion (e.g., paddles and/or gripping members of the anchor portion) in a closed position or configuration after the device has been deployed or implanted in the native heart.

In FIGS. 95-98, a portion of the device 700 with a paddle portion 706 is shown. The device 700 can have any number of paddle portions 706. For example, the device 700 can have one, two, or three paddle portions 706, with each included paddle portion being configured to engage a native heart valve leaflet.

The device 700 can include any other features for implantable prosthetic devices discussed in the present application or any of the applications that are incorporated herein by reference, and the device 700 can be positioned to engage valve tissue 20, 22 as part of any suitable valve repair system (e.g., any valve repair system disclosed in the present application or any of the applications that are incorporated herein by reference). The various components of the device 700 can be made at any suitable size to accommodate different size patient anatomy.

In some implementations, the device 700 extends from a proximal portion 701 to a distal portion 702 and can include an optional coaptation portion 704 and one or more paddle portions 706. In some implementations, the coaptation portion 704 can include a coaptation element 720 (e.g., spacer, coaption element, gap filler, membrane, sheet, plug, wedge, and balloon etc.) for implantation between the leaflets 20, 22 of the native valve. The coaptation element 720 can include any features for spacers or coaptation elements discussed in the present application or any of the applications that are incorporated herein by reference.

In some implementations, the coaptation element 720 includes an outer surface 722 with a first or distal retaining hinge 724 extending outwardly from the outer surface 722 and disposed near the distal portion 702 of the device 700 and a second or proximal retaining hinge 726 extending outwardly from the outer surface 722 and disposed proximally from the first retaining hinge 724 between the proximal and distal portions 701, 702 of the device 700. The second retaining hinge 726 can be disposed above (proximally) and aligned with the first retaining hinge 724 and the first and second retaining hinges 724, 726 can be configured to pivotably retain a portion of the paddle portion 706 of the device 700, as described below.

In some implementations, the first and second retaining hinges 724, 726 each define a circular passage which can receive a tube or shaft and permit the tube or shaft to rotate within the passage. The first and second retaining hinges 724, 726 can be flexible to allow the paddle attachment portion 706 to rotate during delivery and deployment as well as to maintain the paddle portions 706 in a closed position or configuration. While the illustrated retaining hinges 724, 726 are disposed beyond the outer surface 722, the retaining hinges 724, 726 can be part of or disposed within the outer surface 722.

In some implementations, as illustrated, the cross-section of the optional coaptation element 720 is substantially cylindrical. However, the coaptation element 720 can have any suitable size, shape, or configuration. For example, the coaptation element 720 can be any of the spacers or coaptation elements described in the present application and/or the coaptation element 720 can be narrower, such as the size of a small shaft. When included, the coaptation element 720 can have oval, d-shaped, rounded d-shaped, cross-section(s) that mimic the shape of a native valve, etc. Further, the first and second retaining hinges 724, 726 can be disposed on a strut or frame of the device 700 instead of on the coaptation element 720.

In some implementations, each paddle portion 706 of the device 700 includes a paddle 708 with a paddle arm 730 and a slider or follower arm 740. The paddle arm 730 can include a first paddle member 732 and a second paddle member 734 extending at an angle from the first paddle member 732. The paddle arm 730 can also include a paddle fastener 736 at the junction between the first and second paddle members 732, 734 that is configured to be pivotally coupled and/or otherwise retained in the first retaining hinge 724.

In some implementations, the paddle arm 730 can be pivotally coupled or otherwise connected to the first retaining hinge 724 via the paddle fastener 736 such that the paddle fastener 736 is retained in the first retaining hinge 724 and that the first and second paddle members 732, 734 can rotate or pivot about the first retaining hinge 724 and/or the paddle fastener 736. The paddle fastener 736 can take a wide variety of different forms. For example, the paddle fastener 736 can be a shaft and/or a bearing that fits within the first retaining hinge 724. In some implementations, the paddle fastener is integrally formed with one or both of the first and second paddle members 732, 734.

In some implementations, the paddle arm 730 also includes a stop 738 disposed along a length of the first paddle member 732 away from the second paddle member 734 and the paddle fastener 736 which can stop, abut, or otherwise prevent an object from sliding farther along the first paddle member 732. The stop 738 can take a variety of different forms. In some implementations, the stop 738 is a cross bar, a protrusion or projection, a fastener, such as a screw or nut, a weldment, etc.

In some implementations, the first and second paddle members 732, 734 are continuous and are formed by bending the paddle arm 730. In some implementations, the first and second paddle members 732, 734 are separate pieces that are connected together or coupled together. When the paddle members 732, 734 are fixed together, an angle between the paddle members can be between 90 degrees and 160 degrees. However, the paddle members 732, 734 can be set at any angle or can be movable relative to one another in some implementations.

In some implementations, the paddle fastener 736 can be perpendicularly connected or fastened between the first and second paddle members 732, 734, such as by being integrally formed, welding, fasteners, adhesives, etc., and such that the paddle fastener 736 can extend through the first retaining hinge 724 and such that the first and second paddle members 732, 734 can pivot or rotate about the paddle fastener 736 and/or the first retaining hinge 724.

In some implementations, the follower arm 740 can be substantially linear. In some implementations, the follower arm can have a follower fastener 742 at one end and a paddle connector 744 at the end opposite the follower fastener 742. The follower fastener 742 can take a wide variety of different forms. For example, the follower fastener 742 can be a shaft and/or a bearing that fits within the second retaining hinge 726. In some implementations, the follower fastener 742 is integrally formed with the follower arm 740.

In some implementations, the follower arm 740 can optionally be or optionally be configured to act as a spring, such as a leaf spring. In some implementations, the follower fastener 742 is configured to be pivotally coupled or otherwise retained in the second retaining hinge 726 such that the follower arm 740 is connected to the second retaining hinge 726 and can rotate or pivot about the second retaining hinge 726 and/or the follower fastener 742. In some implementations, the paddle connector 744 slidably connects or otherwise fastens to the paddle arm 730 along the length of the first paddle member 732 between the paddle fastener 736 and the stop 738. In some implementations, the paddle connector 744 of the follower arm 740 is size, shaped, or otherwise configured to slide along the first paddle member 732 between the paddle fastener 736 and the stop 738. In some implementations, the paddle connector 744 is a loop which is large enough to slidably fit over a portion of the first paddle member 732 but smaller than the stop 738. However, the paddle connector 744 can have another suitable configuration. For example, the paddle connector 744 can be a tongue which fits and slides within a groove or slot in the first paddle member 732. The follower arm 740 can optionally be sized, shaped, or configured to provide a biasing or spring force which can maintain the paddle arm 730 in a closed position, as described below.

In some implementations, the paddle arm 730 and the follower arm 740 can be configured such that the paddle arm 730 can pivot or rotate freely about the first retaining hinge 724 during delivery and deployment. Additionally, the paddle arm 730, follower arm 740, and the first and second retaining hinges 724, 726 can be sized, shaped, spaced, and configured such that the follower arm 740 and the second paddle member 734 can exert a biasing force to hold the first paddle member 732 in a closed position, such as around the native leaflets 20, 22 when the device 700 is deployed, as described below.

In some implementations, an actuation element 712 extends through the coaptation element 720 and is attached or coupled to the second paddle member 734 of the paddle arm 730 opposite the first paddle member 732. The actuation element 712 can take a wide variety of different forms (e.g., as a wire, rod, shaft, tube, screw, suture, line, strip, combination of these, etc.), be made of a variety of different materials, and have a variety of configurations. In some implementations, the actuation element 712 includes a proximal actuation portion 714 pivotably connected to a distal actuation portion 716 at an actuation pivot 715. In some implementations, the distal end of the distal actuation portion 716 can be pivotably connected to the second paddle member 734 opposite the first paddle member 732 and the paddle fastener 736 such that the distal actuation portion 716 and the second paddle member 734 can rotate relative to each other.

In the extended configuration illustrated by FIG. 95, the actuation element 712 extends through the device 700 with the distal actuation portion 716 substantially in line with the proximal actuation portion 714 and with the paddle arm 730 in position such that the second paddle member 734 is angled toward the proximal actuation portion 714 of the actuation element 712. For example, the second paddle member 734 can be angled proximally into the coaptation element 720 proximally above the first retaining hinge 724.

Referring to FIG. 96, the actuation element 712 can be extended distally through the device 700. As the actuation element 712 is extended distally, because the length of the second paddle member 734 is substantially fixed or fixed, the paddle arm 730 pivots or rotates about the first retaining hinge 724 via the paddle fastener 736 toward the proximal portion 701 of the device and the distal actuation portion 716 can pivot or rotate about the actuation pivot 715. As the paddle arm 730 pivots proximally toward the proximal portion 701 of the device 700, the paddle connector 744 of the follower arm 740 slides along the first paddle member 732 toward the stop 738. Further distal extension of the actuation element 712 causes the pivoting distal actuation portion 716 to rotate the second paddle member 734 about the second retaining hinge 726 and thereby cause the first paddle member 732 to rotate proximally toward the proximal portion 701 of the device 700 until the paddle connector 744 abuts the stop 738 of the paddle arm 730 as shown in FIG. 96.

In some implementations, when the paddle connector 744 of the follower arm 740 abuts the stop 738 of the paddle arm 730, an increased force is required to further pivot the first paddle member 732 about the first retaining hinge 724 toward the proximal portion 701 of the device 700. That is, one or more of the proximal hinge portion 726, the distal hinge portion 724, the proximal hinge portion 726 the paddle arm 730, and the follower arm 740 must flex to allow further closing rotation of paddle arm 730 and the follower arm. Once sufficient distal force is exerted via the actuation element 712, the distal hinge portion 724, the proximal hinge portion 726, the paddle arm 730, and/or the follower arm 740 flex or bend such that the first paddle member 732 can rotate farther toward the proximal portion 701 of the device 700.

FIG. 97 illustrates a center position where an imaginary line passes through the paddle connector 744/stop 738, the pivot axis of the proximal hinge 726, and the pivot axis of the distal hinge 724. In some implementations where the paddle arm 730 and the follower arm 740 are straight, such as in the example illustrated by FIG. 97, the paddle arm 730, the follower arm 740, the pivot axis of the hinge 726, and the pivot axis of the hinge 724 are all aligned in the center position. To reach the center position, the first paddle member 732 is proximally rotated beyond the abutment of the stop 738 and the distal hinge portion 724, the proximal hinge portion 726, the paddle arm 730, and/or the follower arm 740 are flexed, elastically deformed, or compressed a maximum amount. In some implementations, the force required to rotate the paddle arm 730 to the center position is at a maximum or substantially a maximum.

At the center point or at a point just beyond the center point (i.e., an over-center point), the biasing force of the flexed distal hinge portion 724, proximal hinge portion 726, paddle arm 730, and/or the follower arm 740 cause the paddle arm 730 and the follower arm 740 to snap inward or toward the optional coaptation element 720. As such, the biasing force of the distal hinge portion 724, the proximal hinge portion 726, the paddle arm 730, and/or the follower arm 740 cause the first paddle member 732 to remained positioned on the closed side of the over-center point as illustrated by FIG. 98. In some implementations, the paddle arm 730 remains in the closed position until an amount of force is exerted upon the paddle arm 730 by the actuation element 712 that is greater than the amount of force required to move the paddle arm 730 and the follower arm 740 in the opposite direction back over the center position.

After the first paddle member 732 and the follower arm 740 are rotated or moved past the center position, the first paddle member 732 and the follower arm 740 are further pivoted about the first and second retaining hinges 724, 726, respectively, by the biasing force(s) such that the first paddle member 732 and the follower arm 740 are oriented proximally toward the proximal portion 701 of the device 700. After the first paddle member 732 is rotated proximally past the over-center position, the distal hinge portion 724, the proximal hinge portion 726, the paddle arm 730, and/or the follower arm 740 bias the first paddle member 732 toward a medial portion of the device 700. In some implementations, the paddle arm 730 is maintained in the closed position after the first paddle member 732 is moved past the over-center position without the need for any additional lock or application of an external force. The paddle arm 730 can be moved to the over-center and closed positions after native tissue has been properly positioned between the first paddle member 732 and the coaptation element 720.

In some implementations, the bias force provided by first and second retaining hinges 724, 726, the follower arm 740, and/or the abutment of the paddle connector 744 with the stop 738 can provide a locking effect, such as a snap-lock effect, which maintains the paddle arm 730 in the closed position. Without additional force exerted on the paddle arm 730, the first paddle member 732 is maintained in the closed position. For example, when the paddle arm 730 is in the closed position, the biasing forces of the first and second retaining hinges 724, 726, the follower arm 740, and the abutment of the paddle connector 744 with the stop 738 can maintain or lock the first paddle member 732 in the closed position until a force is exerted upon the second paddle member 734 by the actuation element 712 sufficient to rotate the paddle arm 730 and the follower arm 740 back the over the center position.

In some implementations, to re-open the paddle arm 730, sufficient force can be applied to proximally retract the actuation element 712 to overcome the biasing force of the follower arm 740, the first and second retaining hinges 724, 726, and/or the abutment of the paddle connector 744 with the stop 738 and move the paddle arm 730 and follower arm 740 to and over the center position. After the paddle arm 730 and the follower arm 740 are moved past the over-center position, the actuation element 712 can be further retracted to distally rotate the paddle arm 730 farther such that the first paddle member 732 rotates past alignment with the follower arm 740 and such that the paddle connector 744 separates from the stop 738. The paddle arm 730 can be further rotated about the first retaining hinge 724 by further proximal retraction of the actuation element 712 through the device.

As shown in FIG. 95, the device 700 can be deployed in a fully elongated position with the actuation element 712 within the device 700 and the first paddle member 732 oriented substantially distally from the device 700. The second paddle member 734 can be oriented proximally and the follower arm 740 can be disposed with the paddle connector 744 connected to the first paddle member 732 at a distance from the stop 738. In such a position, the cross-sectional profile of the device 700 can be minimal for delivery of the device 700, such as via a delivery system as described above. In some implementations, the first paddle member 732 can be oriented at a 180° angle distally away from the device 700. The follower arm 740 is also oriented distally from the second retaining hinge 726.

In some implementations, as illustrated, the first paddle member 732 is oriented substantially distally from the device 700 during delivery. However, the paddle arm 730 can be oriented in other positions and configurations during delivery. For example, the first paddle member 732 can be rotated beyond 180° distally from the device 700 and can be oriented distally from the device 700 and medially toward the longitudinal axis of the actuation element 712 or the first paddle member 732 can be oriented proximally from 180° from the device 700 during deployment.

As shown in FIG. 96, the actuation element 712 can be advanced distally through the device 700 to rotate the paddle arm 730. The distal actuation portion 716 rotates about the actuation pivot 715 from the proximal actuation portion 714 and rotates the second paddle member 734 about the first retaining hinge 724, thereby rotating the first paddle member 732 about the first retaining hinge 724. In some implementations, the follower arm 740 is connected to the first paddle member 732 and rotates with the first paddle member 732. The paddle arm 730 and the follower arm 740 rotate with the paddle connector 744 sliding along the first paddle member 732 until the paddle connector 744 of the follower arm 740 abuts the stop 738 of the first paddle member 732.

As shown in FIG. 97, additional force can be applied to further distally advance the actuation element 712 through the device 700 to further rotate the paddle arm 730 such that the paddle arm 730 and follower arm 740 are in the center position. In some implementations, the force applied to the actuation element 712 can compress or flex the distal hinge portion 724, the proximal hinge portion 726, the paddle arm 730, and/or the follower arm 740 flex or bend as the paddle arm 730 and follower arm 740 rotate. In some implementations, the distal hinge portion 724, the proximal hinge portion 726, the paddle arm 730, and/or the follower arm 740 can be flexed or compressed a maximum distance when the paddle arm 730 and the follower arm 740 are aligned (i.e., to the over-center position).

As shown in FIG. 98, the actuation element 712 can be pulled farther distally through the device 700 by the biasing force of the distal hinge portion 724, the proximal hinge portion 726, the paddle arm 730, and/or the follower arm 740 as the biasing force further rotates the paddle arm 730 beyond the over-center point. In some implementations, the first paddle member 732 can be retained or locked in the closed position by the biasing effect of the follower arm 740 and the first and second retaining hinges 724, 726 until a sufficient force is exerted on the paddle arm 730 to rotate the first paddle member 732 back past the over-center point. If the device is properly secured on the native valve leaflets, the actuation element 712 can be removed, leaving the device 700 secured to the native valve leaflets.

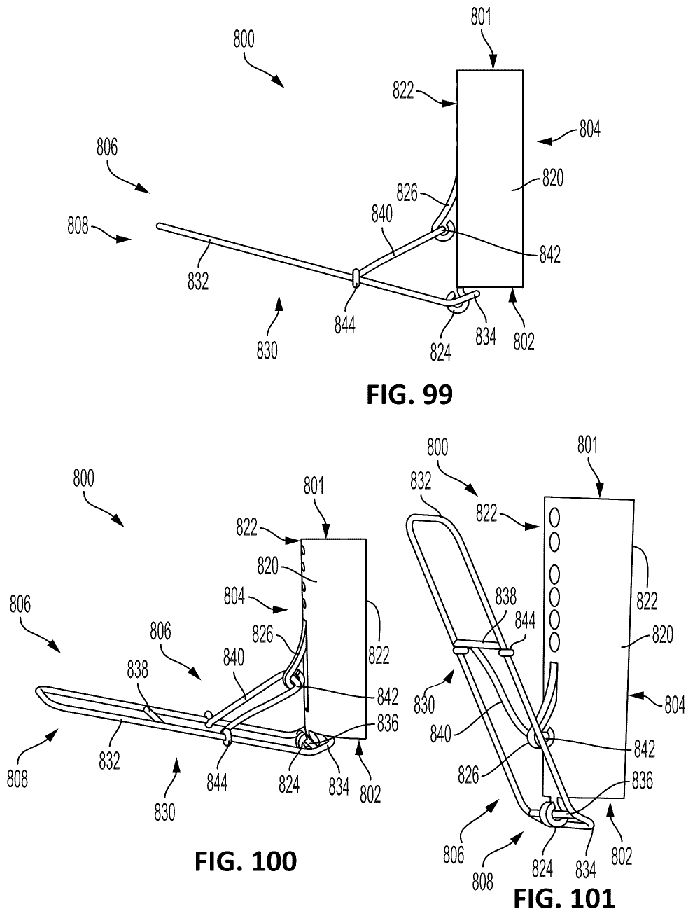

Referring now to FIGS. 99 through 103, an example of an implantable device or implant 800 is shown. The implantable device 800 is one of the many different configurations that the device 700 that is schematically illustrated in FIGS. 95-98 can take. The device 800 can include any other features for an implantable device or implant discussed in the present application, and the device 800 can be positioned to engage valve tissue 20, 22 as part of any suitable valve repair system (e.g., any valve repair system disclosed in the present application). The device/implant 800 can be a valve repair device, implantable device, or another type of implant that attaches to leaflets of a native valve.

In some implementations, the device 800 extends from a proximal portion 801 to a distal portion 802 and can include an optional coaptation portion 804 and anchor portion. In some implementations, the anchor portion comprises one or more paddle portions 806. In some implementations, the anchor portion can optionally comprise an attachment portion or gripping members similar to those described elsewhere herein. In some implementations, the coaptation portion 804 can include a coaptation element 820 (e.g., spacer, coaption element, gap filler, membrane, sheet, plug, wedge, and balloon etc.) for implantation between the leaflets 20, 22 of the native valve. The coaptation element 820 can include any of the features for spacers or coaptation elements discussed in the present application or any of the applications that are incorporated herein by reference.

In some implementations, the coaptation element 820 includes an outer surface 822 with a first or distal retaining hinge 824 extending outwardly from the outer surface 822 and disposed near the distal portion 802 of the device 800 and a second or proximal retaining hinge 826 extending outwardly from the outer surface 822 and disposed proximally from the first retaining hinge 824 between the proximal and distal portions 801, 802 of the device 800. In some implementations, the second retaining hinge 826 can be disposed above (proximal to) the first retaining hinge 824 and the first and second retaining hinges 824, 826 can be configured to pivotably retain a portion of the paddle portion 806 of the device 800, as described below. In some implementations, the first and second retaining hinges 824, 826 each define a circular passage which can receive a tube or shaft and permit the tube or shaft to rotate within the passage. In some implementations, the first and second retaining hinges 824, 826 can be flexible to allow the paddle portions 806 to rotate after the stop is reached during delivery and deployment as well as to maintain the paddle portions 806 in a closed position or configuration.

In some implementations, as illustrated, the coaptation element 820 is substantially cylindrical. However, the coaptation element 820 can have any suitable size, shape, or configuration. For example, the coaptation element 820 can be any of the spacers or coaptation elements described in the present application and/or the coaptation element 820 can be narrower, such as the size of a small shaft. Further, the first and second retaining hinges 824, 826 can be disposed on a strut or frame of the device 800 instead of on the coaptation element 820.

In some implementations, each paddle portion 806 of the device 800 includes a paddle 808 with a paddle arm 830 and a slide or follower arm 840. In some implementations, the paddle arm 830 includes a first paddle member 832 and a second paddle member 834 extending at an angle from the first paddle member 832. In some implementations, as illustrated, the paddle arm 830 comprises a wire or tube that is bent into a substantially rectangular loop shape that is bent to form the first paddle member 832 and the second paddle member 834 such that the first and second paddle members 832, 834 are substantially U-shaped with end portions and two legs extending from the end. However, the paddle arm 830 can have other suitable sizes, shapes, and configurations. For example, the paddle arm 830 can be oval, elliptical, or hourglass shaped, can comprise radial flares extending inwardly or outwardly, can be curved at a proximal end of the first paddle member 832, and can be formed from mechanical linkages.

In some implementations, the paddle arm 830 also includes a paddle fastener 836 at the junction between the first and second paddle members 832, 834 that is configured to be secured or otherwise retained in the first retaining hinge 824. In some implementations, the paddle arm 830 can be fastened or otherwise connected to the first retaining hinge 824 via the paddle fastener 836 such that the paddle fastener 836 is retained in the first retaining hinge 824 and that the first and second paddle members 832, 834 can rotate or pivot about the first retaining hinge 824 and/or the paddle fastener 836. In some implementations, the paddle fastener 836 can rotatably connect to the first retaining hinge 824 such that the first and second paddle members 832, 834 can rotate about the first retaining hinge 824. In some implementations, as illustrated, the paddle fastener 836 is a rod or shaft extending between the legs of the first and second paddle members 832, 834 and is optionally integral with the first and second paddle members 832, 834. However, the paddle fastener 836 can have another size, shape, or configuration. For example, the paddle fastener 836 can comprise connector rods which snap-fit into the ends of the first retaining hinge 824 or can comprise a mechanical linkage which pivotably connects the paddle arm 830 with the first retaining hinge 824.

In some implementations, the paddle arm 830 also includes a stop 838 disposed along a length of the first paddle member 832 away from the second paddle member 834 and the paddle fastener 836 configured to stop, abut, or otherwise prevent an object from sliding farther along the first paddle member 832. In some implementations, as illustrated, the stop 838 is a rod or shaft extending between the legs of the first paddle member 832. However, the stop 838 can have other sizes, shapes, or configurations. For example, the stop 838 can be a projection along a length of the first paddle member 832 or the wall or end of a groove along a length of the first paddle member 832 which prevents an object from sliding farther along the length of the first paddle member 832. In some implementations, as illustrated, the stop 838 is a rod or shaft extending between the legs of the first paddle member 832 and secured between the legs of the first paddle member 832, such as by welding, connectors, fasteners, or adhesives.

In some implementations, the follower arm 840 can optionally be configured to act as a biasing element or spring, such as a leaf spring. In some implementations, the follower arm 840 is substantially U-shaped with one end comprising a follower fastener 842, two legs extending from the follower fastener 842, and paddle connectors 844 at the ends of each leg opposite the follower fastener 842. In some implementations, the follower fastener 842 is configured to be secured or otherwise retained in the second retaining hinge 826 such that the remainder of the follower arm 840 can rotate or pivot about the second retaining hinge 726 and/or the follower fastener 842. In some implementations, the paddle connectors 844 slidably connect or otherwise fasten to the paddle arm 830 along the length of the first paddle member 832 between the paddle fastener 836 and the stop 838.

In some implementations, the paddle connectors 844 are looped portions of the follower arm 840 which are large enough to slidably fit over a portion of the first paddle member 832 but which may not slide past the stop 838. However, the paddle connectors 844 can have other suitable configurations. For example, the paddle connectors 844 can be tongues which fit and slide within grooves or slots in the first paddle member 832. In some implementations, the follower arms 840 can be sized, shaped, or configured to optionally provide biasing or spring forces which can maintain the paddle arm 830 in a closed position or assist in maintaining the paddle arm 830 in the closed position.

The paddle arm 830 and the follower arm 840 can each comprise steel or a shape memory alloy, such as Nitinol—produced in a wire, sheet, tubing, or laser sintered powder—and can be configured such that the paddle arm 830 and follower arm 840 can pivot or rotate freely about the first retaining hinge 824 during delivery and deployment. Additionally, the paddle arm 830, follower arm 840, and the first and second retaining hinges 824, 826 can be sized, shaped, spaced, and configured such that the paddle arm 830, the follower arm 840, the first retaining hinge 824, and/or second retaining hinge 826 exert a biasing force to hold the first paddle member 832 in a closed position, such as around the native leaflets 20, 22 when the device 800 is deployed.

As shown in FIGS. 99 through 104, the device 800 can be moved between an open position and a closed position, similarly to device 700. In some implementations, the device 800 can be deployed in a configuration with the first paddle member 832 oriented substantially distally from the coaptation element 820 and the second paddle member 834 can be rotated distally away from the coaptation element 820 such that the first paddle member 832 rotates proximally toward the coaptation element 820 (FIGS. 99-100). In some implementations, the paddle connectors 844 can slide along the legs of the first paddle member 832 as the first paddle member 832 rotates, thereby proximally rotating the follower arm 840. In some implementations, the first paddle member 832 can be freely rotated until the paddle connectors 844 of the follower arm 840 abut the stop 838 of the first paddle member 832 (FIG. 101).

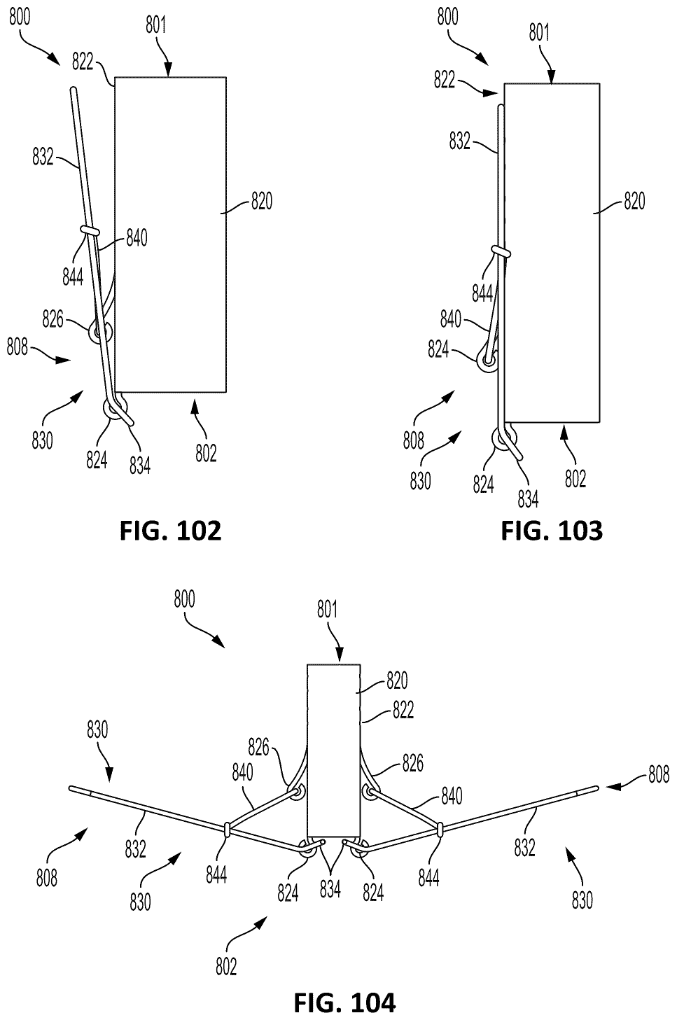

In some implementations, by exerting additional pressure on the second paddle member 834, the paddle arm 830 and follower arm 840 can be rotated to a center position in which the paddle arm 830 and first paddle member 832 are substantially aligned. In the center position, the paddle arm 830, the follower arm 840, the first retaining hinge 824, and/or second retaining hinge 826 is/are flexed, elastically deformed, or compressed a maximum amount (FIG. 102). In some implementations, a maximum amount of force can be applied to the second paddle member 834 to move the paddle arm 830 and the follower arm 840 to the past or over the center position.

In some implementations, the biasing force applied by the paddle arm 830, the follower arm 840, the first retaining hinge 824, and/or second retaining hinge 826 rotate and lock the second paddle member 834 and the first paddle member 832 in the closed position, past or over the center position. For example, the paddle arm 830 can be moved to the over-center position and closed after native heart valve tissue, such as native heart valve leaflets, has been properly placed between the paddle arm 830 and the coaptation element 820 (FIG. 103). In the closed position, the first paddle member 832 can abut and/or be biased towards or against the coaptation element 820.

Referring now to FIGS. 104-106, more than one paddle portion 806 can be included on a device 800. Each paddle 808 has a paddle arm 830 and a follower arm 840. In some implementations, each paddle arm 830 can be pivotally coupled in a first retaining hinge 824 and each follower arm 840 can be secured or otherwise retained in a second retaining hinge 826 as described regarding FIGS. 96-98 and 99-104. In some implementations, the paddles 808 and first and second retaining hinges 824, 826 can be evenly spaced and disposed on substantially opposite sides of the device 800 such that the device 800 can be secured to native leaflets on multiple sides of the device 800. In some implementations, as illustrated, the device 800 has two paddles 808 disposed on opposite sides of the device 800. However, the device 800 can include any number of paddles 808, paddle arms 830, and follower arms 840 secured or otherwise connected to first and second retaining hinges 824, 826. For example, the device can include three paddles 808, such as for use in a tricuspid valve, or four or more paddles 808.

In some implementations, each paddle 808 can be rotated by an actuation element and moved between an open position and a closed position, as described above regarding FIGS. 95-98 and 99-104. In some implementations, the device 800 can be delivered and deployed with the first paddle members 832 of each paddle arm 730 disposed substantially distally from the device 800. In some implementations, the paddle arms 730 can then be proximally rotated to an open position, rotated to an over-center position, and rotated to a closed position beyond the over-center position by an actuation element, as described above. The paddles 808 can be actuated independently or in concert.

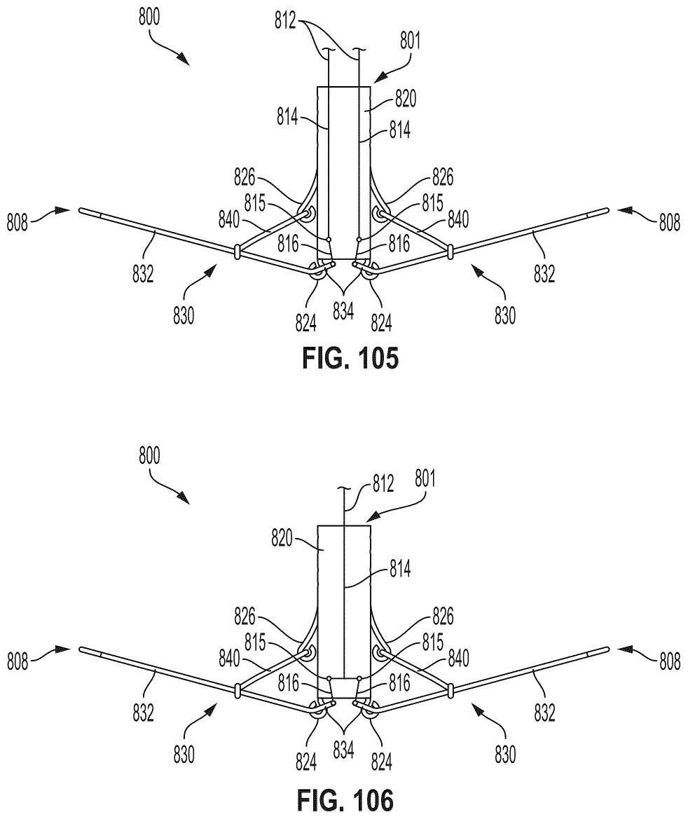

As shown in FIG. 105, the device 800 can be configured such that the paddles 808 can be actuated and moved between the open, center, and closed positions independently. Two actuation elements 812 each having a proximal actuation portion 814 pivotably connected to a distal actuation portion 816 at an actuation pivot 815 extend through the coaptation element 820. In some implementations, the distal actuation portion 816 of each actuation element 812 pivotably connects to one of the second paddle members 834 opposite the first paddle member 832 and the paddle fastener 836. Each actuation element 812 can be independently actuated as described regarding actuation element 712 in FIGS. 95-98 to move each paddle 808 between the open and closed positions. For example, a user can extend or retract the proximal actuation portion 814 of either actuation element 812 through the device 800 to move the respective paddle 808 between the open, center, and closed positions independently from the other paddle 808.

As shown in FIG. 106, the device 800 can be configured such that the paddles 808 can be actuated and moved between the open, over-center, and closed positions simultaneously. In some implementations, an actuation element 812 having a proximal actuation portion 814 and two distal actuation portions 816 pivotably connected to the proximal actuation portion 814 at actuation pivots 815 extends through the coaptation element 820. In some implementations, each distal actuation portion 816 is pivotably connected to one of the second paddle members 834 opposite the first paddle member 832 and the paddle fastener 836. In some implementations, the actuation element 812 can be actuated as described regarding actuation element 712 in FIGS. 95-98 to simultaneously move both paddles 808 between the open and closed positions. For example, a user can extend or retract the proximal actuation portion 814 of the actuation element 812 through the device 800 to simultaneously move both paddles 808 between the open, over-center, and closed positions.

In some implementations, as illustrated, the proximal actuation portion 814 is T-shaped such that each distal actuation portion 816 pivotably connects to the proximal actuation portion 814 via a separate actuation pivot 815. However, the actuation element 812 can have other configurations. For example, both distal actuation portions 816 can be pivotably connected to the proximal actuation portion 814 at a single actuation pivot 815.

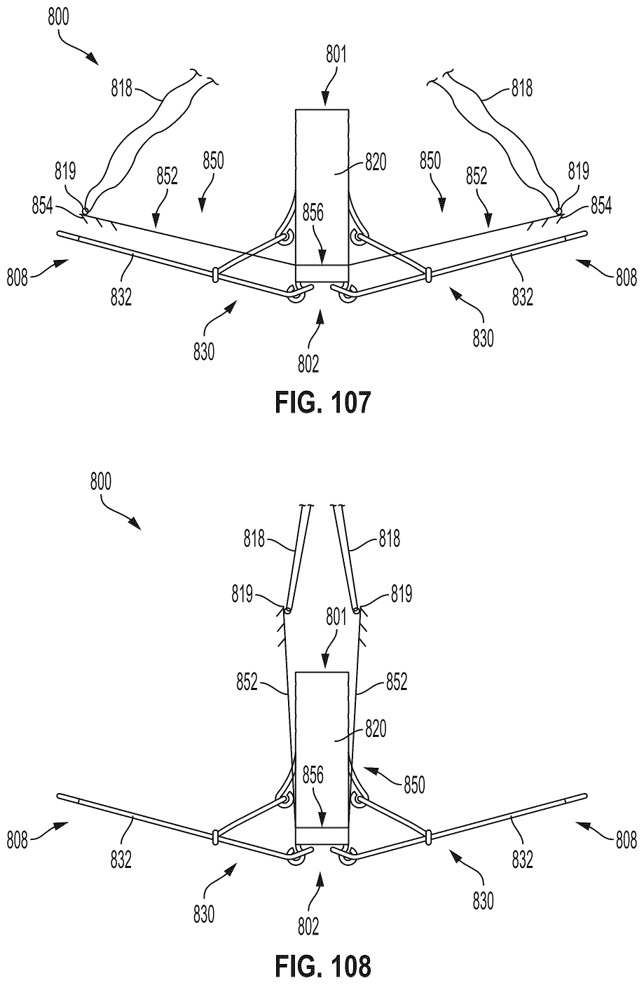

Referring now to FIGS. 107-110, the paddle portions 806 and/or paddles 808 can include attachment portions or gripping members (e.g., gripping arms, clasp arms, etc.) 850 that can be moved between open and closed positions. The device 800 can include a number of gripping members 850 corresponding to the number of paddles 808.

In some implementations, as illustrated, the gripping members 850 can comprise a moveable arm 852 and optional friction-enhancing elements or other securing structures 854 (e.g., barbs, protrusions, ridges, grooves, textured surfaces, adhesive, etc.). In some implementations, the moveable arms 852 can be biased to a normally closed position with the moveable arms 852 oriented distally toward the first paddle members 832. In some implementations, the moveable arms 852 can optionally be spring loaded so that in the closed position, the gripping members 850 continue to provide a pinching force on the grasped native leaflet. Optional barbs, friction-enhancing elements, or securing structures 854 of the gripping members 850 can grab, pinch, and/or pierce the native leaflets to further secure the native leaflets.

In some implementations, the gripping members 850 can be opened by applying tension to actuation lines 818 attached to the moveable arms 852, thereby causing the moveable arms 852 to articulate, flex, or pivot away from the first paddle members 832. In some implementations, the actuation lines 818 can extend through a delivery system (e.g., through a steerable catheter and/or an implant catheter) and can connect to the moveable arms 852 at a loop 819 disposed through or otherwise connected to an outer portion of the moveable arms 852. In some implementations, the actuation lines 818 can take a wide variety of forms, such as, for example, a line, a suture, a wire, a rod, a catheter, or the like.

In some implementations, during implantation, the paddles 808 can be opened and closed, for example, to grasp the native leaflets (e.g., the native mitral valve leaflets, etc.) between the paddles 808 and/or between the paddles 808 and the coaptation element 820. The gripping members 850 can be used to grasp and/or further secure the native leaflets by engaging the leaflets with optional barbs, friction-enhancing elements, or securing structures 854 and pinch the leaflets with the moveable arms 852. In some implementations, the optional barbs, friction-enhancing elements, or other structures 854 (e.g., protrusions, ridges, grooves, textured surfaces, adhesives, etc.) of the gripping members 850 increase friction with the leaflets or can partially or completely puncture the leaflets.

In some implementations, the actuation lines 818 can be actuated separately so that each gripping member 850 can be opened and closed separately. Separate operation allows one leaflet to be grasped at a time, or for the repositioning of a gripping member 850 on a leaflet that was insufficiently grasped, without altering a successful grasp on the other leaflet. In some implementations, the gripping members 850 can be opened and closed relative to the position of the first paddle member 832 (as long as the first paddle member 832 is in an open or at least partially open position), thereby allowing leaflets to be grasped in a variety of positions as the particular situation requires.

As shown in FIGS. 107-108, the gripping members 850 can include a collar 856 which can connect or secure the moveable arms 852 to the coaptation element 820. The collar 856 can be sized and shaped to be secured, placed, or otherwise disposed on the coaptation element 820. For example, the collar 856 can be rounded or elliptical and be sized and shaped to be at least partially snap fit into a clasp securing recess of the coaptation element 820 or to be at least partially secured in the clasp securing recess by an interference fit. In some implementations, the collar 856 can provide a spring or bias force to the moveable arms 852 which biases the moveable arms 852 toward a closed position with the moveable arm 852 biased distally toward the first paddle member 832. In some implementations, the collar 856 can include any suitable joint with the moveable arms 852, such as a flexible joint, a spring joint, a pivot joint, or the like. In some implementations, the joint between the collar 856 and the moveable arm 852 is a flexible piece of material integrally formed with the collar 856 and moveable arms 852.

As shown in FIG. 107, when the paddle arms 830 of the device 800 are in the open position and without tension applied to the actuation lines 818, the moveable arms 852 are biased toward the first paddle member 832 by the collar 856 and/or a hinge connection between the collar 856 and the moveable arms 852. As shown in FIG. 108, tension can be applied to the actuation lines 818 connected to the moveable arms 852 which causes the moveable arms 852 to articulate, flex, or pivot on the joint between the moveable arms 852 and the collar 856. In some implementations, the tension applied to the actuation lines 818 cause the gripping members 850 to move to the open position. For example, the gripping members 850 can be moved to the open position to properly position native tissue between the moveable arms 852 and the first paddle members 832. Releasing the tension in the actuation lines 818 can cause the moveable arms 852 to move back to the closed position. For example, tension in the actuation lines 818 can be released when native tissue is properly positioned between the gripping members 850 and the first paddle members 832 such that the moveable arms 852 close and securely retain the native tissue between the moveable arms 852 and the first paddle member 832. While the illustrated gripping members 850 are simultaneously moved between the open and closed position, it will be understood that the moveable arm 852 of each gripping member 850 can be independently moved between the opened and closed positions.

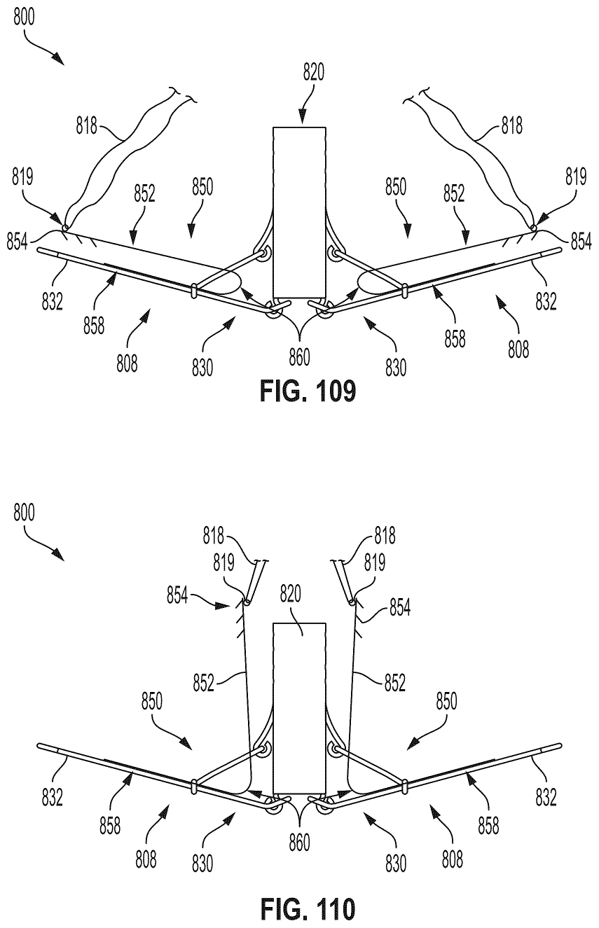

As shown in FIGS. 109-110, in some implementations the gripping members 850 of the device can each include a base or fixed arm 858 and a joint portion 860. In some implementations, the fixed arms 858 are attached to the first paddle members 832 with the joint portion 860 disposed proximate the coaptation element 820. In some implementations, the joint portion 860 can provide a spring force between the fixed and moveable arms 858, 852 of the gripping members 850. In some implementations, the spring force provided by the joint portion 860 can bias the moveable arms 852 to a closed position with the moveable arms 852 distally toward the first paddle member 832.

In some implementations, the joint portion 860 can be any suitable joint, such as a flexible joint, a spring joint, a pivot joint, or the like. In some implementations, the joint portion 860 is a flexible piece of material integrally formed with the fixed and moveable arms 858, 852. In some implementations, the fixed arms 858 are attached to the first paddle members 832 and remain station or substantially stationary relative to the first paddle members 832 when the moveable arms 852 are opened to open the gripping members 850 and expose the optional barbs, friction-enhancing elements, or securing structures.

As shown in FIG. 109, when the paddle arms 830 of the device 800 are in the open position and without tension applied to the actuation lines 818, the moveable arms 852 are biased toward the fixed arms 858 by the joint portions 860. As shown in FIG. 110, tension can be applied to the actuation lines 818 connected to the moveable arms 852 which causes the moveable arms 852 to articulate, flex, or pivot on the joint portions 860.

In some implementations, the tension applied to the actuation lines 818 can cause the gripping members 850 to move to the open position. For example, the gripping members 850 can be moved to the open position to properly position native tissue between the moveable arms 852 and fixed arms 858 and/or the first paddle members 832. Releasing the tension in the actuation lines 818 causes the moveable arms 852 to move back to the closed position. For example, tension in the actuation lines 818 can be released when native tissue is properly positioned between the moveable arms 852 and the fixed arms 858 and/or the first paddle members 832 such that the moveable arms 852 close and securely retain the native tissue between the moveable arms 852 and the fixed arms 858 and/or the first paddle member 832. While the illustrated gripping members 850 are simultaneously moved between the open and closed position, it will be understood that the moveable arm 852 of each gripping member 850 can be independently moved between the opened and closed positions.

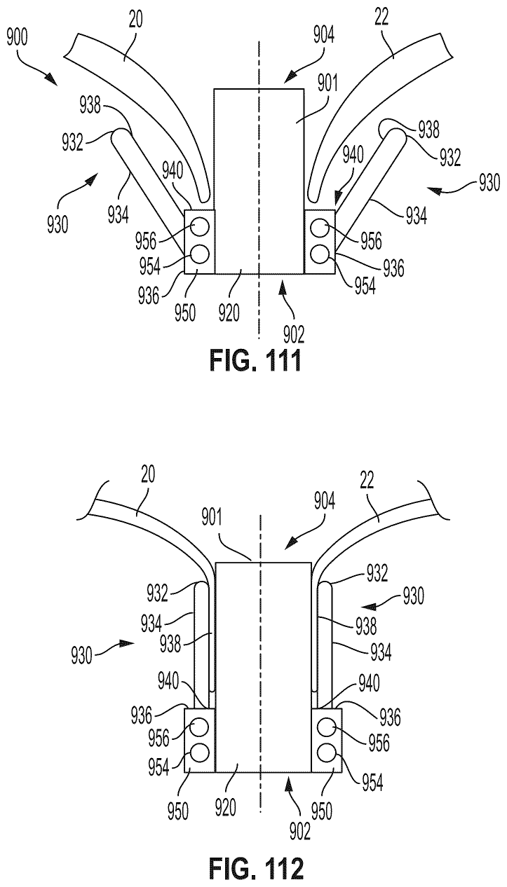

Referring to FIGS. 111 and 112, an example of a device (e.g., an implantable prosthetic device, a prosthetic spacer device, a valve repair device, etc.) 900 is schematically illustrated. The device 900 can include any other features for implantable prosthetic devices discussed in the present application or any of the applications that are incorporated herein by reference, and the device 900 can be positioned to engage valve tissue 20, 22 as part of any suitable valve repair system (e.g., any valve repair system disclosed in the present application or any of the applications that are incorporated herein by reference).

In some implementations, the device 900 extends from a proximal portion 901 to a distal portion 902 and can include an optional coaptation portion 904 and anchor portion. In some implementations, the anchor portion can comprise one or more paddle portions 906. In some implementations, the anchor portion can optionally comprise an attachment portion or gripping members similar to those described elsewhere herein. In some implementations, the coaptation portion 904 can include a coaptation element (e.g., spacer, coaption element, gap filler, membrane, sheet, plug, wedge, and balloon etc.) for implantation between the leaflets 20, 22 of the native valve. The coaptation element 920 can include any features for spacers or coaptation elements discussed in the present application or any of the applications that are incorporated herein by reference.

In some implementations, as illustrated, the cross-section of the optional coaptation element 920 is substantially cylindrical. However, the coaptation element 920 can have any suitable size, shape, or configuration. For example, the coaptation element 920 can be any of the spacers or coaptation elements described in the present application and/or the coaptation element 920 can be narrower, such as the size of a small shaft.

In some implementations, the paddle portion 906 of the device includes one or more paddles 908 with a paddle arm 930 and a paddle arm connector 950 configured to permit the paddle arm 930 to rotate freely when the paddle arm 930 is disposed in a first, unbiased, or rotatable position or configuration in the paddle arm connector 950 and to provide a biasing force which can prevent the paddle arm 930 from rotating when the paddle arm 930 is disposed in a second, biased, or locked position or configuration in the paddle arm connector 950. In some implementations, as illustrated, the device 900 includes two paddles 908 disposed on substantially opposite sides of the device 900. However, the device 900 can include any number of paddles 908. For example, the device can include one paddle 908, three paddles 908, such as for use in a tricuspid valve, or four or more paddles 908.

In some implementations, the paddle arm 930 can be substantially U-shaped with an end portion 932, a first leg portion 934 extending from one side of the end portion 932, and a second leg portion 938 extending from the opposite side of the end portion 932. In some implementations, the first leg portion 934 can include a first coupling portion 936 at the end of the first leg portion 934 opposite the end portion 932 and the second leg portion 938 can include a second coupling portion 940 at the end of the second leg portion 938 opposite the end portion 932. In some implementations, the first and second coupling portions 936, 940 can be configured to be pivotably connected, disposed in, or otherwise attached to the paddle arm connector 950 such that the end portion 932 can rotate about the first and second coupling portions 936, 940.

In some implementations, the paddle arm 930 comprises a wire, tube, shaft, etc. that is bent substantially into a U-shape to form the end portion 932, the first and second leg portions 934, 938, and the first and second coupling portions 936, 940. However, the paddle arm 930 can have other suitable sizes, shapes, and configurations. For example, the paddle arm 930, such as the end portion 932 and the first and second leg portions 934, 938 can be oval, elliptical, or hourglass shape, can comprise radial flares extending inwardly or outwardly, can be curved at a proximal end near the end portion 932, or can be formed from mechanical linkages. Further, the paddle arms 930 can comprise steel or a shape memory alloy, such as Nitinol—produced in a wire, sheet, tubing, or laser sintered powder—and can be configured to provide a biasing force against rotation of the paddle arm 930 about the paddle arm connector 950 when the paddle arm 930 is in the biased or locked position.

In some implementations, the paddle arm connector 950 can be coupled to or otherwise disposed on the coaptation portion 904, such as on coaptation element 920. In some implementations, the first and second coupling portions 936, 940 can be pivotably connected to or disposed in the paddle arm connector 950 such that the paddle arm 930 can be freely rotated about the paddle arm connector 950 and/or the first and second coupling portions 936, 940, toward the coaptation element 920 when the first and/or second coupling portions 936, 940 of the paddle arm 930 are in the unbiased position. In some implementations, the position of the first and/or second coupling portions 936, 940 in a biased position in the paddle arm connector 950 causes a biasing force, such as a leaf spring biasing force in the paddle arm itself, that acts against rotation of the paddle arm 930.

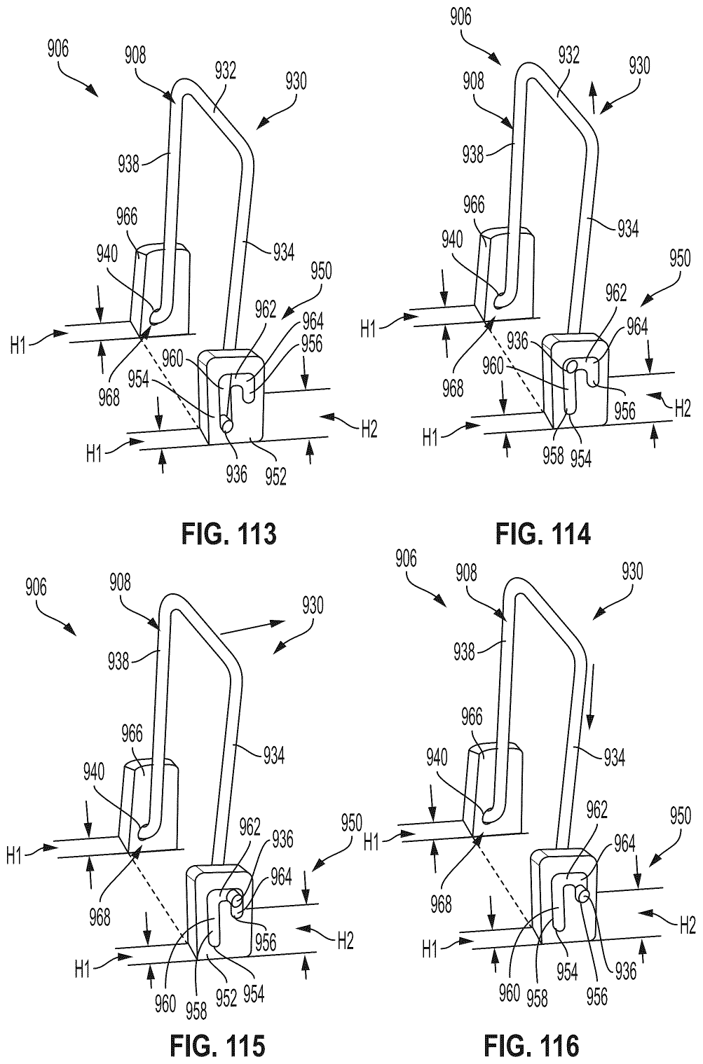

In some implementations, the paddle arm connector 950 can include a first receiving portion 954 for receiving or retaining the first coupling portion 936 when the paddle arm 930 is in the unbiased position and a second receiving portion 956 for receiving or retaining the first coupling portion 936 when the paddle arm 930 is in the biased position. In some implementations, the second coupling portion 940 can be pivotably received in a fixed retaining portion of the paddle arm connector 950 which is substantially in line with the first receiving portion 954 and offset from the second receiving portion 956 (FIGS. 113-116). Referring to FIG. 113, the paddle arm 930 can freely rotate when the paddle arm 930 is in the unbiased position as the first coupling portion 936 is in the first receiving portion 954 and substantially aligned with the second coupling portion 940.

Referring to FIGS. 114-116, the first coupling portion 936 can be moved from the first receiving portion 954 to the second receiving portion 956 such that the paddle arm 930 is moved to the biased position. In some implementations, when the first coupling portion 936 is disposed in the second receiving portion 956, the first and second coupling portions 936, 940 are offset and the difference in rotational axes for the first and second leg portions 934, 938 (e.g., the first and second coupling portions 936, 940) provide a biasing force preventing the paddle arm 930 from rotating. That is, the misalignment between the first coupling portion 936 and the second coupling portion 940 inhibits the paddle arm 930 from pivoting without flexing or bending the paddle arm. In some implementations, the force required to flex or bend the paddle arm 930 biases the paddle arm back to the closed position, so long as the paddle arm is not plastically deformed by the flexing or bending.

As shown in FIG. 111, the device 900 can be deployed or otherwise moved to a substantially open position with the paddle arms 930 oriented away from the coaptation element 920. In some implementations, the paddle arms 930 can be disposed in the unbiased or rotatable position with the first coupling portion 936 of the first leg portion 934 rotatingly disposed in the first receiving portion 954 of the paddle arm connector 950. In the first position, the paddle arms 930 can be freely rotated.

As shown in FIG. 112, the paddle arms 930 can be rotated or actuated from the open position to a closed position proximally toward the coaptation element 920, such as around the native leaflets 20, 22 when the device 900 is deployed, as described below. In some implementations, the paddle arms 930 can be rotated via an actuation element (e.g., actuation shaft, actuation wire, etc.), actuation lines (e.g., line, suture, wire, rod, catheter, etc.), or any other manner described in the present application or any of the applications that are incorporated herein by reference. In some implementations, the first coupling portion 936 can then be moved from the first receiving portion 954 to the second receiving portion 956, thereby moving the paddle arm 930 into the biased or locked position. For example, the first coupling portion 936 can be moved from the first receiving portion 954 to the second receiving portion 956 once the native leaflets 20, 22 have been properly positioned between the paddles 908 and the coaptation element 920.

While the illustrated paddles 908 are simultaneously moved between the open and closed position, it will be understood that the paddle arm 930 of each paddle 908 can be independently moved between the opened and closed positions. Further, each paddle 908 can be independently moved between the unbiased or rotatable position and the biased or locked position. Further, in some implementations, the coaptation element 920 can include one or more slots or passages extending through a body of the coaptation element 920 such that one or more actuation elements can extend through a central portion of the device 900 and attach or otherwise couple to the paddles 908, such as the end portions 932 of the paddle arms 930.

As shown in FIGS. 113-116, each paddle 908 can be configured such that the paddle arm 930 can be moved between the unbiased and biased positions in the paddle arm connector 950. In some implementations, the paddle arm connector 950 can include a paddle channel 958 connecting the first and second receiving portions 954, 956. In some implementations, the paddle channel 958 is configured to allow the first coupling portion 936 of the first leg portion 934 to be moved between the first and second receiving portions 954, 956, such as to move the paddle arm 930 between the unbiased and biased configurations. In some implementations, the paddle channel 958 can be configured such that the first coupling portion 936 can be retained in the first or second receiving portions 954, 956 until acted upon by an external force, such as by a user with an actuation element. While the illustrated paddle channel 958 is substantially L-shaped, it will be understood that the paddle channel 958 can have other shapes. For example, the paddle channel 958 can have an arcuate shape, serpentine shape, zig-zag shape, etc. Any shape that allows the first coupling portion 936 to be moved between first (unbiased or unlocked) and second (biased or locked) stable positions (i.e. the position stays the same unless an external force is applied) where the first coupling portion 936 is aligned with the second connecting portion in the first (unbiased or unlocked) stable position and the first coupling portion 936 is offset from the second connecting portion in the second (biased or locked) stable position.

In some implementations, the first and second coupling portions 936, 940 are bent or otherwise angled outwardly from the first and second leg portions 934, 938, respectively, such that the first coupling portion 936 can be received in a first paddle connector 952 of the paddle arm connector 950 and the second coupling portion 940 can be received in a second paddle connector 966 of the paddle arm connector 950 opposite the first paddle connector 952. In some implementations, the first coupling portion 936 can be angled away from the first and second leg portions 934, 938 and received in the paddle channel 958 of the first paddle connector 952, such as in the first or second receiving portions 954, 956. In some implementations, the second coupling portion 940 can be angled away from the first and second leg portions 934, 938 and received in a fixed retaining portion 968 extending inwardly or through the second paddle connector 966. In some implementations, the fixed retaining portion 968 can be substantially aligned with the first receiving portion 954 of the first paddle connector 952 and can be configured to retain the second coupling portion 940 and permit the second coupling portion 940 to rotate about a fixed axis extending through the fixed retaining portion 968. For example, the fixed retaining portion 968 can be a bore, aperture, or passage extending into or through the second paddle connector 966.

In some implementations, the fixed retaining portion 968 can be positioned or configured to permit the paddle arm 930 to rotate relatively freely when the first coupling portion 936 is disposed in the first receiving portion 954 (unbiased position) and to provide a biasing force to prevent the paddle arm 930 from rotating when the first coupling portion 936 is disposed in the second receiving portion 956 (biased position). For example, the fixed retaining portion 968 can be substantially aligned with the first receiving portion 954 and radially and laterally offset from the second receiving portion 956.

In some implementations, the fixed retaining portion 968 can be disposed in the second paddle connector 966 at a first height H1 from a bottom of the paddle arm connector 950 (e.g., the portion connected to the coaptation element 920) and the first receiving portion 954 can be disposed in the first paddle connector 952 at the first height H1 from the bottom of the paddle arm connector 950. In some implementations, the second receiving portion 956 can be disposed in the first paddle connector 952 at a second height H2 above the bottom of the paddle arm connector 950. In some implementations, the difference between the first and second heights H1, H2 can be such that the difference in the axis of rotation of the first leg portion 934 when the paddle arm 930 is in the biased configuration (e.g., the first coupling portion 936 in the second receiving portion 956) can be sufficiently offset from the axis of rotation of the second leg portion 938 (e.g., the second coupling portion 940 in the fixed retaining portion 968) that rotation of the paddle arm 930 is inhibited or restricted.

In some implementations, the second receiving portion 956 can also be laterally offset from the fixed retaining portion 968. For example, the axes of rotation of the first and second leg portions 934, 938 when the paddle arm 930 is in the unbiased position (e.g., the second coupling portion 940 in the fixed retaining portion 968 and the first coupling portion 936 in the first receiving portion 954) can be substantially aligned such that the paddle arm 930 can freely rotate about the shared axes and the axes of rotation of the first and second leg portions 934, 938 when the paddle arm 930 is in the biased position (e.g., the second coupling portion 940 in the fixed retaining portion 968 and the first coupling portion 936 in the second receiving portion 956) can be offset such that the paddle arm 930 and/or the paddle arm connector 950 exert a biasing force which prevents or otherwise restricts rotation of the paddle arm 930.

While the illustrated first and second coupling portions 936, 940 are substantially perpendicular to the first and second leg portions 934, 938, the first and second coupling portions 936, 940 can have other sizes, shapes, and configurations, such as to further secure the first and second coupling portions 936, 940 in the paddle arm connector 950. For example, the first and second coupling portions 936, 940 can include a projection, flange, or curve disposed at an end of the first and second coupling portions 936, 940 (opposite the leg portions 934, 938) on the opposite side of the paddle channel 958 and the fixed retaining portion 968 from the first and second leg portions 934, 938 such that the first and second coupling portions 936, 940 may not be retracted through or from the paddle channel 958 or the fixed retaining portion 968, respectively.

In some implementations, the paddle channel 958 is configured such that the first coupling portion 936 can be retained in the first receiving portion 954 at the first height H1, moved to the second receiving portion 956, such as via an actuation element, and retained in the second receiving portion 956 at the second height H2. In some implementations, the paddle channel 958 can include a first channel portion 960 extending upwardly (e.g., toward the coaptation element 920) from the first receiving portion 954, a second channel portion 962 extending laterally from an end of the first channel portion 960 opposite the first receiving portion 954, and a third channel portion 964 extending downwardly (e.g., away from the coaptation element 920) from an end of the second channel portion 962 opposite the first channel portion 960 and extending to the second receiving portion 956.

In some implementations, the first channel portion 960 can extend from the first receiving portion 954 at the first height H1 substantially vertically or proximally to a height greater than the second height H2. In some implementations, the second channel portion 962 can extend substantially laterally from the top of the first channel portion 960. In some implementations, the third channel portion 964 can extend downwardly or distally from the second channel portion 962 to the second height H2. In some implementations, the third channel portion 964 can have a length between the second channel portion 962 and the second receiving portion 956 such that the first coupling portion 936 can be retained in the second receiving portion 956 until an upward or proximal force is exerted on the first leg portion 934, such as via an actuation element.

In some implementations, the device 900 can be deployed with the paddle arm 930 of each paddle 908 in the first or unbiased position with the first coupling portion 936 of the first leg portion 934 disposed in the first receiving portion 954 of the paddle arm connector 950 (FIG. 113). In some implementations, the first coupling portion 936 of the first leg portion 934 of the paddle arm 930 can be disposed in the first receiving portion 954 of the paddle arm connector 950 at the first height H1 and substantially aligned with the second coupling portion 940 of the second leg portion disposed in the fixed retaining portion 968 at the first height H1. In some implementations, the first and second leg portions 934, 938 of the paddle arm 930 can be substantially free to pivot or rotate about a common rotation axis extending through the first and second coupling portions 936, 940. For example, the device 900 can be deployed with the paddles 908 in the open position extending outwardly from the coaptation element 920, such that the paddle arm 930 can be actuated to grasp native leaflets 20, 22.

In some implementations, the paddle 908 can then be moved from the unbiased or rotatable position to the biased or lock position, such as by applying one or more forces to the paddle arm 930. For example, the forces can be applied via an actuation element. A force can be applied to the paddle arm 930, such as upward or proximal force applied at the end portion 932, first leg portion 934, and/or second leg portion 938, to move the first coupling portion 936 upwardly from the first receiving portion 954 along the length of the first channel portion 960 to a height of the second channel portion 962 above the second height H2 (FIG. 114). A force can then be applied to the paddle arm 930, such as a lateral force to move the first coupling portion 936 laterally along the length of the second channel portion 962 such that the first coupling portion 936 is aligned with the third channel portion 964 (FIG. 115). A force can then be applied to the paddle arm 930, such as a downward force (e.g., toward the coaptation element 920) to move the first coupling portion 936 downwardly along the length of the third channel portion 964 from the height of the second channel portion 962 to the second receiving portion 956 (FIG. 116).

In some implementations, the paddle 908 can be in the biased or locked position when the first coupling portion 936 is disposed in the second receiving portion 956 (FIG. 116). In some implementations, the first coupling portion 936 of the first leg portion 934 of the paddle arm 930 can be disposed at the second height H2 in the second receiving portion 956 and the second coupling portion 940 of the second leg portion 938 can be disposed at the first height H1 in the fixed retaining portion 968. In some implementations, the first coupling portion 936 disposed in the second receiving portion 956 can also be laterally offset from the second coupling portion 940 disposed in the fixed retaining portion 968. As such the pivot or rotational axis of the first leg portion 934 (the first coupling portion 936) is offset from the pivot or rotational axis of the second leg portion 938 (the second coupling portion 940) and the configuration of the paddle arm 930 and/or the paddle arm connector 950 provide a biasing force, such as a leaf spring biasing force, that inhibits or otherwise restrict the paddle arm 930 from rotating.

In some implementations, the paddle 908 can also be moved from the biased or locked position to the unbiased or rotatable position. For example, when the first coupling portion 936 is disposed in the second receiving portion 956 (FIG. 116), an outward force can be applied to the paddle arm 930 to move the first coupling portion 936 upwardly to the top of the third channel portion 964 (FIG. 115), a lateral force can be applied to the paddle arm 930 to move the first coupling portion 936 along the second channel portion 962 to the top of the first channel portion 960 (FIG. 114), and an inward force can be applied to the paddle arm 930 to move the first coupling portion 936 downwardly along the first channel portion 960 to the first receiving portion 954.

While the movements and forces are described in relative terms such as upwardly, downwardly, laterally, outwardly, and inwardly, it will be understood that the directions of the movements and forces can be different based upon the position and orientation of the paddle 908. For example, movements or forces described as being up or down can be lateral and movements or forces described as being lateral can be up or down when the paddle 908 is disposed on the device 900, such as on the coaptation element 920.

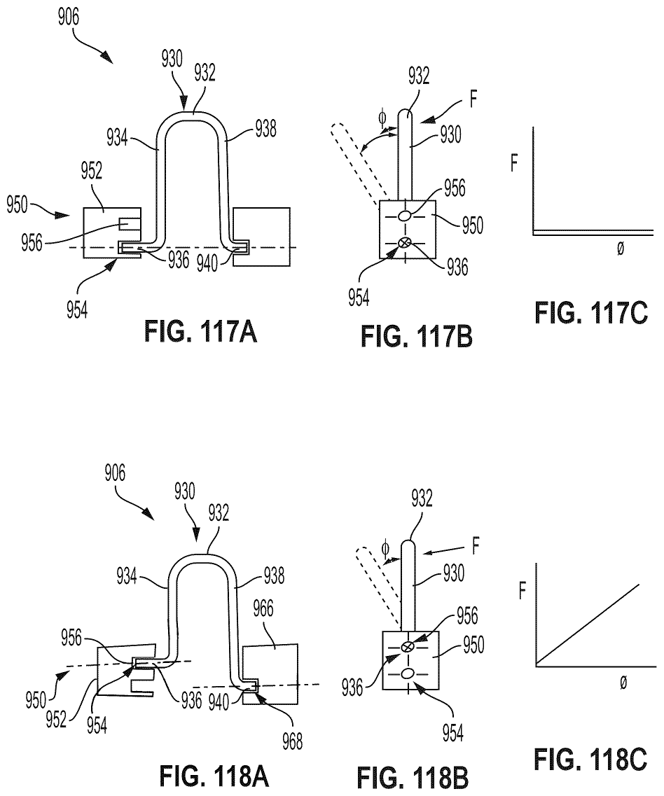

FIGS. 117A-117C illustrate the amount of force applied to the paddle arm 930 when the first coupling portion 936 of the paddle arm 930 is disposed in the first receiving portion 954 (unbiased position) to rotate the paddle arm 930 an angle (about the paddle arm connector 950. As shown, the first coupling portion 936 disposed in the first receiving portion 954 is substantially aligned with the second coupling portion 940 disposed in the fixed retaining portion 968. As a force F is applied laterally to the end portion 932 of the paddle arm 930, the paddle arm 930 can rotate an angle (about the rotational axes of the first and second coupling portions 936, 940. As the first and second coupling portions 936, 940 are substantially aligned, the force F required to rotate the paddle arm 930 is zero or negligible as the angle (increases. For example, the force F required to rotate the paddle arm 930 can increase slightly or negligibly due to frictional forces between the first coupling portion 936 and the first receiving portion 954 and between the second coupling portion 940 and the fixed retaining portion 968. As such, the paddle arm 930 can be substantially free to rotate about the paddle arm connector 950.

FIGS. 118A-118C illustrate the force exerted on the paddle arm 930 when the first coupling portion 936 of the paddle arm 930 is disposed in the second receiving portion 956 (biased position) and the paddle arm 930 is rotated an angle (about the paddle arm connector 950. As shown, the first coupling portion 936 disposed in the second receiving portion 956 is offset from the second coupling portion 940 disposed in the fixed retaining portion 968. As a force F is applied laterally to the end portion 932 of the paddle arm 930, the paddle arm 930 can rotate an angle (about the rotational axes of the first and second coupling portions 936, 940. As the first and second coupling portions 936, 940 are offset, the rotational axis of the first leg portion 934 (e.g., the first coupling portion 936) is offset from the rotational axis of the second leg portion 938 (e.g., the second coupling portion 940). As such, the paddle arm connector 950 and/or the configuration of the paddle arm 930 provide a bias against rotation of the paddle arm 930. In some implementations, the force required to rotate the paddle arm 930 increases as the paddle arm 930 is rotated farther, such as by resisting deformation of the paddle arm 930. Accordingly, the force F required to farther rotate the paddle arm 930 can increase as the angle of rotation (increase. For example, the force F required to rotate the paddle arm 930 can increase proportionally as the angle of rotation (increases. As such, the paddle arm 930 can be inhibited, restricted or locked from rotating about the paddle arm connector 950.

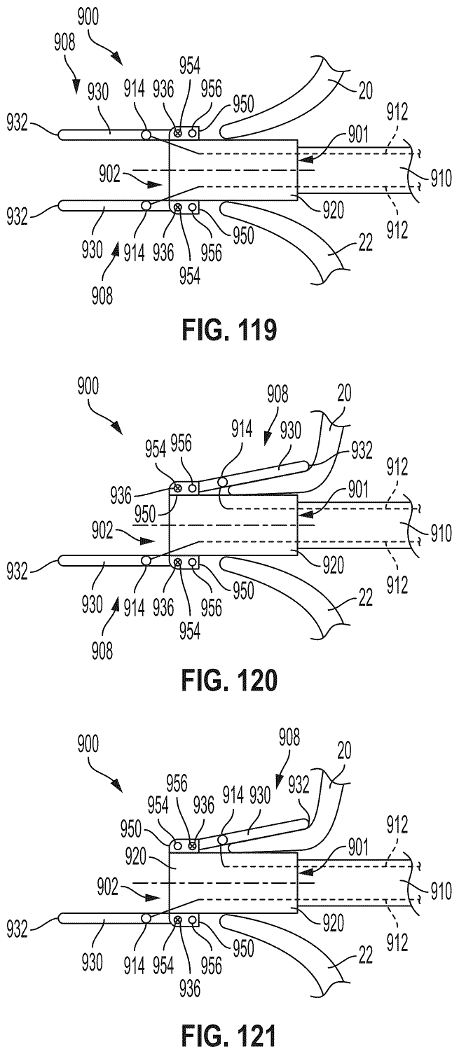

Referring now to FIGS. 119-124, the device 900 can be deployed or implanted within the native heart, such as implanted between the leaflets 20, 22 of the native valve. The device 900 can be connected to a delivery system 910. In some implementations, the delivery system 910 can comprise one or more of a catheter, a sheath, a guide catheter/sheath, a delivery catheter/sheath, a steerable catheter, a tube, a channel, a pathway, combinations of these, etc. In some implementations, the delivery system 910 can be configured to position the device 900, close the device to capture one or more native valve leaflets, lock the device closed, and release the device 900 from the delivery system.

In some implementations, the delivery system 910 can include one or more actuation elements 912 (e.g., actuation line, actuation wire, actuation shaft, etc.) extending through the delivery system (e.g., guide catheter/sheath, etc.) and connected to one or more of the paddles 908, such as to the paddle arms 930. In some implementations, the actuation elements can extend through the coaptation element 920, such as through a slot or passage in the body of the coaptation element 920, to connect to the paddle 908. In some implementations, the actuation elements 912 can be connected to the paddles 908 at a connection portion 914, such as a loop, hook, or other connective component which can releasably connect with the paddle 908 and/or the actuation element 912.

As shown in FIG. 119, the device 900 can be deployed from the delivery system 910 with the paddles 908 in the open and unbiased positions. For example, the paddle arms 930 can extend distally away from the coaptation element 920 and the paddle arm connector 950 and the first coupling portion 936 of each paddle arm 930 can be disposed in the first receiving portion 954 of the paddle arm connector 950. As such, the paddle arms 930 can be freely rotated, such as by actuation of one of the actuation elements 912. While the illustrated paddle arms 930 are oriented substantially distally from the coaptation element 920 when the device 900 is in the open position, the paddle arms 930 can have other orientations in the open position. For example, the paddle arms 930 can be rotated more than 180° from the coaptation element 920 or the paddle arms 930 can be rotated less than 180° from the coaptation element 920 in the open position.

As shown in FIG. 120, one of the paddles 908 can be moved from the open position to the closed position. For example, the paddle 908 can be moved to the closed position to capture one of the leaflets 20, 22 between the paddle arm 930 and the coaptation element 920. The actuation element 912 can be actuated, such as by a user, to rotate the paddle arm 930 about the paddle arm connector 950. For example, the actuation element 912 can be retracted proximally through the device 900 and the delivery system 910 to rotate the paddle arm 930 about the paddle arm connector 950 toward the coaptation element 920.

As shown in FIG. 121, the paddle 908 in the closed position can be moved from the unbiased position to the biased position, such as to bias or lock the paddle arm 930 in place. For example, the paddle 908 can be moved to the biased position once the leaflet 20, 22 has been properly positioned between the paddle arm 930 and the coaptation element 920. The actuation element 912 can be actuated, such as by a user, to move the first coupling portion 936 of the paddle arm 930 from the first receiving portion 954 of the paddle arm connector 950 to the second receiving portion 956 of the paddle arm connector 950. For example, the actuation element 912 can exert the forces described in FIGS. 113-116 to move the first coupling portion 936 from the first receiving portion 954 of the paddle arm connector 950 to the second receiving portion 956 of the paddle arm connector 950. In some implementations, the second coupling portion 940 of the second leg portion 938 of the paddle arm 930 (can remain in the fixed retaining portion 968 (see FIG. 118). As such, the offset alignment between the first and second coupling portions 936, 940 can exert a biasing force on the paddle arm 930 which substantially prevents or restricts rotation of the paddle arm 930, thereby locking the paddle 908 in place.

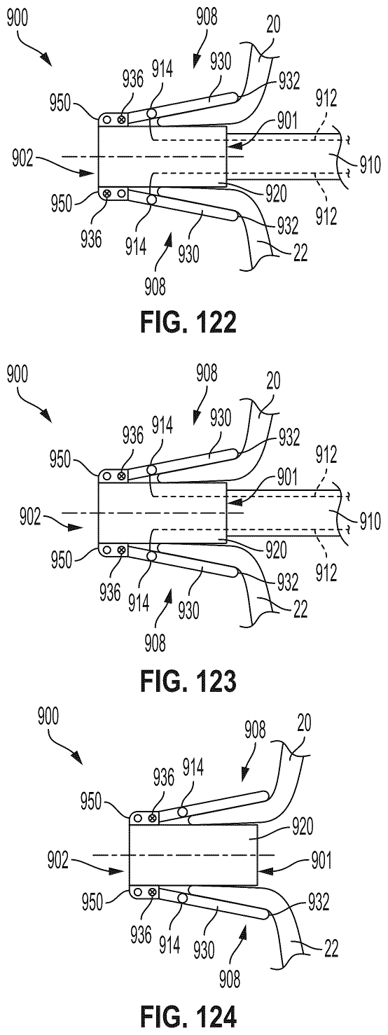

As shown in FIG. 122, the second paddle 908 can be moved from the open position to the closed position, such as to capture the second leaflet 20, 22 between the paddle arm 930 of the second paddle 908 and the coaptation element 920. For example, the paddle arm 930 of the second paddle 908 can be rotated about the paddle arm connector 950 from the open position to the closed position as described above in FIG. 120.

As shown in FIG. 123, the second paddle 908 can be moved from the unbiased position to the biased position, such as to bias the second paddle 908 after the second leaflet 20, 22 has been properly positioned between the second paddle 908 and the coaptation element 920. For example, the first coupling portion 936 of the paddle arm 930 of the second paddle 908 can be moved from the first receiving portion 954 to the second receiving portion 956 of the paddle arm connector 950 as described above in FIG. 121.

As shown in FIG. 123, the device 900 can be in a fully closed and deployed condition. The delivery system 910 and the actuation elements 912 are retracted and the paddles 908 remain in a fully closed and biased (locked) positions. For example, the connection portions 914 of the actuation elements 912 can be decoupled from the paddles 908 and/or the actuation elements 912 such that the actuation elements 912 can be retracted from the device 900. Once deployed, the device 900 can be maintained in the fully closed position with the biasing force exerted by the offset rotational axes of the first and second leg portions 934, 938 of the paddle arm 930 disposed in the paddle arm connector 950 preventing the paddles 908 from reopening. Similarly, the configuration of the paddle arms 930 in the biased position can exert a bias which pinches the leaflets 20, 22.

While the device 900 has been illustrated as actuating the two paddles 908 separately to capture the leaflets 20, 22, it will be understood that the paddles 908 can be actuated and locked simultaneously. For example, the paddle arm 930 of each paddle 908 can be coupled to a single actuation element, such as an actuation element similar to actuation element 812 in FIG. 106, such that the paddles 908 can be moved in concert from the open position to the closed position to capture the leaflets 20, 22 and moved from the unbiased position to the biased position to lock the paddles 908 in place.

Further, the concepts of the device 900 can be combined with any of the individual components of the disclosed devices and systems described in the present application or any of the applications that are incorporated herein by reference. For example, the device 900 can include any of the attachment portions, clasps, or gripping members (e.g., gripping arms, clasp arms, etc.) that can be moved between open and closed positions and which can include frictional-enhancing elements or other securing structures (e.g., barbs, protrusions, ridges, grooves, textured surfaces, adhesives, etc.), as described above.

The above method(s) can be performed on a living animal or on a simulation, such as on a cadaver, cadaver heart, anthropomorphic ghost, simulator (e.g., with the body parts, heart, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—can be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures can be described herein as being a preferred arrangement or method, such description is not intended to suggest that such feature is required or necessary unless expressly so stated. Still further, example or representative values and ranges can be included to assist in understanding the present application, however, such values and ranges are not to be construed in a limiting sense and are intended to be critical values or ranges only if so expressly stated.

Moreover, while various aspects, features and concepts can be expressly identified herein as being inventive or forming part of a disclosure, such identification is not intended to be exclusive, but rather there can be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the treatment techniques, methods, operations, steps, etc. described or suggested herein can be performed on a living animal or on a non-living simulation, such as on a cadaver, cadaver heart, simulator (e.g., with the body parts, tissue, etc. being simulated), etc. The words used in the claims have their full ordinary meanings and are not limited in any way by the description of the examples in the specification.

We claim:

1. A valve repair device for repairing a native valve of a patient, the valve repair device comprising:
a first retaining hinge;
a second retaining hinge disposed proximally to the first retaining hinge;
a paddle comprising:
a paddle arm having a first paddle member with a stop and a paddle fastener rotatably retained in the first retaining hinge;
a follower arm having a follower fastener rotatably retained in the second retaining hinge and a paddle connector slidable along a portion of the first paddle member;
wherein the paddle is rotatable from an open position to a first position in which the paddle connector abuts the stop, a center position in which the paddle arm and the follower arm are substantially aligned, and a closed position; and
wherein at least one of the first retaining hinge, the second retaining hinge, and the follower arm bias the paddle arm to the closed position when the paddle is rotated past the center position.

2. The valve repair device of claim 1, wherein at least one of the first retaining hinge and the second retaining hinge bias the paddle arm to the closed position when the paddle is rotated past the center position.

3. The valve repair device of claim 1, wherein the second retaining hinge biases the paddle arm to the closed position when the paddle is rotated past the center position.

4. The valve repair device of claim 1, wherein the follower arm exerts a leaf spring biasing force on the paddle when the paddle is rotated proximally past the first position.

5. The valve repair device of claim 1 further comprising a coaptation element attached to the first retaining hinge and the second retaining hinge.

6. The valve repair device of claim 1, wherein the first paddle member is a wire loop and the stop comprises a rod disposed between legs of the first paddle member.

7. The valve repair device of claim 1, wherein the paddle further comprises a gripping member with a movable arm that is movable between a closed position and an open position.

8. A valve repair device for repairing a native valve of a patient, the valve repair device comprising:
a base;
a paddle comprising:
a paddle arm with a first leg portion having a first connecting portion and a second leg having a second connecting portion;
a paddle arm connector with a fixed retaining portion for receiving the second connecting portion and first and second receiving portions for receiving the first connecting portion; and
wherein the paddle arm is rotatable about the paddle arm connector when the first connecting portion is disposed in the first receiving portion and is biased against rotation when the first connecting portion is disposed in the second receiving portion.

9. The valve repair device of claim 8, wherein the paddle arm connector includes a channel connecting the first and second receiving portions.

10. The valve repair device of claim 9, wherein the first receiving portion and the fixed retaining portion are disposed at a first height and the second receiving portion is disposed at a second height, the second height being greater than the first height.

11. The valve repair device of claim 9, wherein the channel is L-shaped.

12. The valve repair device of claim 9, wherein the channel further includes a first channel portion extending upwardly from the first receiving portion, a second channel portion extending laterally from the first channel portion, and a third channel portion extending downwardly from an end of the second channel portion opposite the first channel portion to the second receiving portion.

13. A valve repair device for repairing a native valve of a patient, the valve repair device comprising:
a coaptation element formed from a solid or hollow piece of molded material;
a paddle portion having a plurality of paddles that are movable between an open position and a closed position;
an attachment portion having a collar and two clasp elements, each clasp element having a clasp securing recess;
wherein the paddle portion is configured to attach to the native valve of the patient and hold leaflets of the native valve against the attachment portion; and
wherein the plurality of paddles are movable between the open position and the closed position independently.

14. The valve repair device of claim 13 wherein the coaptation element includes two actuators.

15. The valve repair device of claim 14 wherein distal movement of one of the two actuators causes one of the plurality of paddles to move to the open position, and proximal movement of the one actuator causes one of the plurality of paddles to move to the closed position.

16. The valve repair device of claim 13, wherein the paddle portion is secured in paddle securing recesses of the coaptation element.

17. The valve repair device of claim 13, further comprising a biasing element which biases one of the plurality of paddles to one of the open position and the closed position.

18. The valve repair device claim 13, further comprising a connection element configured to move one of the plurality of paddles between the opened and closed positions.

19. The valve repair device of claim 18, wherein the paddle portion includes a connection portion for connecting to the connection element.

20. The valve repair device of claim 18, wherein the paddle portion includes an outer paddle and an inner paddle.
